(19) United States
(12) Patent Application Publication
(10) Pub. No.: US 2020/0380882 A1
(43) Pub. Date: Dec. 3, 2020

(54) COGNITIVE PLATFORM INCLUDING COMPUTERIZED EVOCATIVE ELEMENTS IN MODES

(71) Applicant: Akili Interactive Labs, Inc., Boston, MA (US)

(72) Inventors: Titiimaea Alailima, Cambridge, MA (US); Jeffery Bower, Norwood, MA (US); Walter E. Martucci, Westwood, MA (US); Ashley Mateus, Cambridge, MA (US); Isabella Slaby, Robbinsville, NJ (US); Matthew Omernick, Larkspur, CA (US); Adam Piper, Petaluma, CA (US); Paul Rand Pierce, Seattle, WA (US); Scott Kellogg, Mattapoisett, MA (US); Elena Cañadas Espinosa, Dorchester, MA (US)

(21) Appl. No.: 16/636,181

(22) PCT Filed: Aug. 3, 2018
(86) PCT No.: PCT/US2018/045206

§ 371 Date: Feb. 3, 2020

Related U.S. Application Data
(63) Continuation-in-part of application No. PCT/US2017/045385, filed on Aug. 3, 2017.
(60) Provisional application No. 62/541,080, filed on Aug. 3, 2017.

(51) Int. Cl. G09B 19/00; A61B 5/16; A61B 5/103; A61B 5/00; G06K 9/00; A61B 5/0205

(52) U.S. Cl. CPC G09B 19/00; A61B 5/163; A61B 5/1032; A61B 5/162; A61B 5/024; G06K 9/00308; A61B 5/4836; A61B 5/4848; A61B 5/0205; A61B 5/7264

(57) ABSTRACT

Apparatus, systems and methods are provided for quantifying aspects of cognition (including cognitive abilities) under emotional load. In certain configurations, the apparatus, systems and methods can be implemented for enhancing certain cognitive abilities.

### CROSS-REFERENCE TO RELATED APPLICATIONS

This application claims priority benefit of U.S. Provisional Application No. 62/541,080, entitled “COGNITIVE PLATFORM INCLUDING COMPUTERIZED EVOCATIVE ELEMENTS IN MODES” filed on Aug. 3, 2017, and is a continuation-in-part of U.S. International Application No. PCT/US2017/045385, entitled “COGNITIVE PLATFORM INCLUDING COMPUTERIZED EVOCATIVE ELEMENTS” filed on Aug. 3, 2017, each of which is incorporated herein by reference in its entirety, including drawings.

### BACKGROUND OF THE DISCLOSURE

The ability to make rapid and efficient selection of emotionally relevant stimuli in the environment is crucial for functioning in society. Individuals with the capability of emotion processing have a better capability to flexibly and adaptively respond appropriately in differing situations. Research shows that several differing regions of the brain are involved in emotion processing, and selective attention. The interaction of these regions of the brain act together to extract the emotional or motivational value of sensory events and help an individual respond appropriately in differing situations. Certain cognitive conditions, diseases, or executive function disorders can result in compromised capability for identifying emotionally relevant stimuli and responding appropriately.

### SUMMARY OF THE DISCLOSURE

In view of the foregoing, apparatus, systems and methods are provided for quantifying aspects of cognition (including cognitive abilities) under emotional load. In certain configurations, the apparatus, systems and methods can be implemented for enhancing certain cognitive abilities.

In an embodiment, aspects of the invention relate to a system for generating an indication of cognitive skills in an individual using evocative elements presented in one or more differing modes. The system includes one or more processors, and a memory to store processor-executable instructions and communicatively coupled with the one or more processors. Upon execution of the processor-executable instructions by the one or more processors, the one or more processors are configured to generate a user interface, and present via the user interface a first instance of a primary task in the presence of a secondary task including an interference configured to divert the individual's attention from the first instance of the primary task, requiring a first response from the individual to the first instance of the primary task in the presence of the interference and a secondary response from the individual to the interference. The first instance of the primary task or the interference includes the evocative elements presented in the one or more differing modes including at least one of a first mode wherein the primary task includes two or more evocative elements presented substantially simultaneously at the user interface, or a second mode wherein the interference includes two or more evocative elements presented substantially simultaneously at the user interface. The one or more processors are also configured to receive data indicative of a first response and a secondary response, at least one of the first response and the secondary response including a measure of a physical action of the individual in response to at least one of the evocative elements, wherein the data includes at least one measure of emotional processing capabilities of the individual under emotional load; and to analyze the data indicative of the first response and the secondary response to generate at least one performance metric including at least one quantified indicator of cognitive abilities of the individual under emotional load.

One or more of the following features may be included. The one or more processors may be configured to configure the user interface to instruct the individual not to respond to an evocative element that is configured as a distractor. The one or more processors may be configured to configure the user interface to instruct the individual to respond to an evocative element that is configured as an interruptor.

In another aspect, embodiments of the invention relate to a system for generating an indication of cognitive skills in an individual using evocative elements presented according to one or more integration rules. The system includes one or more processors, and a memory to store processor-executable instructions and communicatively coupled with the one or more processors. Upon execution of the processor-executable instructions by the one or more processors, the one or more processors are configured to generate a user interface, and to present via the user interface a first instance of a primary task in the presence of a secondary task including an interference configured to divert the individual's attention from the first instance of the primary task, requiring a first response from the individual to the first instance of the primary task in the presence of the interference and a secondary response from the individual to the interference. The interference includes a plurality of evocative elements presented according to the one or more integration rules, at least one of the evocative elements being configured as a distractor and at least one of the evocative elements being configured as an interruptor and having either a specified facial expression or a specified non-evocative feature. The one or more integration rules are configured such that the plurality of evocative elements are presented with at least two differing non-evocative features, each non-evocative feature either being correlated with a specific facial expression or not correlated with any facial expressions. The one or more processors are also configured to receive data indicative of the first response and the secondary response, at least one of the first response and the secondary response including a measure of the individual's response to the evocative elements, wherein (i) the response comprises a physical action of the individual in response to the evocative elements and (ii) the data comprises at least one measure of emotional processing capabilities of the individual under emotional load. The one or more processors are configured to analyze the data indicative of the first response and the secondary response to generate at least one performance metric including at least one quantified indicator of cognitive abilities of the individual under emotional load.

One or more of the following features may be included. The one or more processors may be configured to configure the user interface to instruct the individual not to respond to an evocative element that is configured as a distractor. The one or more processors may be configured to configure the user interface to instruct the individual to respond to an evocative element that is configured as an interruptor.

The non-evocative feature is at least one of color or a shape. The one or more integration rules may include a first integration rule that requires each evocative element to be presented with a non-evocative feature that is not correlated with a facial expression, the interruptor including an evocative element having a specified facial expression.

The one or more integration rules may include a second integration rule that requires each evocative element to be presented with a non-evocative feature that is correlated with a facial expression, the interruptor including an evocative element having a specified non-evocative feature.

The one or more integration rules may include a third integration rule that requires each evocative element to be presented with a non-evocative feature that is correlated with a facial expression, the interruptor including an evocative element having a specified facial expression.

The one or more integration rules may include a fourth integration rule that requires each evocative element to be presented with a single non-evocative feature with differing facial expressions based on eyes and/or mouth, the interruptor including an evocative element having a specified facial expression.

Various aspects of the invention may include one or more of the following features. The one or more processors may be are further configured to present via the user interface a second instance of the primary task without the interference, requiring a second response from the individual to the second instance of the primary task; and analyze the data indicative of the first response, the second response, and the secondary response to generate the at least one performance metric.

The primary task may be a continuous visuo-motor task. The interference may be a target discrimination task. At least one of the first response and/or the secondary response may include measured data indicative of the physical action of the individual.

The one or more processors may be further configured to at least one of: (i) generate an output representing the at least one generated performance metric or (ii) transmit to a computing device the at least one generated performance metric.

The one or more processors may be further configured to present via the user interface a second instance of the primary task, requiring a second response from the individual to the second instance of the primary task; and analyze a difference between the data indicative of the first response and the second response to compute an interference cost as a measure of at least one additional indication of cognitive abilities of the individual.

In the first instance, the primary task may be continuous and rendered over a first time interval, in the second instance the primary task may be continuous and rendered over a second time interval, and the first time interval may be different from the second time interval. The measure of cognitive capabilities of the individual may be computed based on at least one of a measure of the individual's capability to distinguish among differing types of evocative elements, and/or a measure of the individual's capability to distinguish among evocative elements having differing valence.

The one or more processors may configure at least one evocative element as a temporally overlapping task with at least one of the first instance of the primary task or the interference.

The one or more processors may be further configured to generate a predictive model based on values of the at least one generated performance metric, to generate a predictive model output indicative of a measure of cognition, a mood, a level of cognitive bias, or an affective bias of the individual.

The predictive model may include at least a linear/logistic regression, principal component analysis, generalized linear mixed models, random decision forests, support vector machines, and/or artificial neural networks.

At least one evocative element may include an image of a face that represents or correlates with an expression of a specific emotion or a combination of emotion

The primary task or the interference may include an adaptive response-deadline procedure having a response-deadline; and the one or more processors may be further configured to modify the response-deadline of the at least one adaptive response-deadline procedure to adjust a difficulty level of the primary task or the interference.

The one or more processors may be configured to control the user interface to modify a temporal length of the response window associated with the response-deadline procedure. Adjusting the difficulty level may include applying an adaptive algorithm to progressively adjust a level of valence of the at least one evocative element

The generated performance metric may include an indicator of a projected response of the individual to a cognitive treatment.

The generated performance metric may include a quantitative indicator of at least one of a mood, a cognitive bias, and/or an affective bias of the individual.

The one or more processors may be further configured to use the at least one first performance metric to at least one of (i) recommend a change of at least one of an amount, concentration, and/or dose titration of a pharmaceutical agent, drug, or biologic, (ii) identify a likelihood of the individual experiencing an adverse event in response to administration of the pharmaceutical agent, drug, or biologic, (iii) identify a change in the individual's cognitive response capabilities, (iv) recommend a treatment regimen, or (v) recommend or determine a degree of effectiveness of at least one of a behavioral therapy, counseling, or physical exercise.

The data may be indicative of the first response and the secondary response at a first difficulty level, and the one or more processors may be further configured to analyze data indicative of the first response and the secondary response at a second difficulty level to generate at least one second performance metric representative of a performance of the individual of interference processing under emotional load.

The one or more processors may be further configured to measure substantially simultaneously the first response from the individual to the first instance of the task, a secondary response of the individual to the interference, and the response to the at least one evocative element, and generate the performance metric based on the first response, secondary response, and the response to the at least one evocative element.

The one or more processors may be further configured to adjust a difficulty of at least one of the task and/or the interference based on the at least one generated first performance metric such that the primary task with the interference are rendered at a second difficulty level; and generate a second performance metric representative of cognitive abilities of the individual under emotional load based at least in part on the data indicative of the first response and the response of the individual to the at least one evocative element.

The system may be at least one of a virtual reality system, an augmented reality system, or a mixed reality system.

One or more physiological components may be included, wherein upon execution of the processor-executable instructions by the one or more processors, the one or more processors receive data indicative of one or more measurements of the physiological component; and analyze the data indicative of the first response and the response of the individual to the at least one evocative element, and the data indicative of one or more measurements of the physiological component to generate the first performance metric.

The one or more physiological components may be configured to measure data indicative of one or more of heart rate, skin conductance, reaction time, and accelerometer measurements.

In another aspect, embodiments of the invention relate to a system for enhancing cognitive skills in an individual using evocative elements presented in one or more differing modes. The system includes one or more processors, and a memory to store processor-executable instructions and communicatively coupled with the one or more processors. Upon execution of the processor-executable instructions by the one or more processors, the one or more processors are configured to generate a user interface, and to present via the user interface, at a first difficulty level in a first iteration, a first instance of a primary task in the presence of a secondary task including an interference configured to divert the individual's attention from the first instance of the primary task, requiring a first response from the individual to the first instance of the primary task in the presence of the interference and a secondary response from the individual to the interference. The first instance of the primary task or the interference includes the evocative elements presented in the one or more differing modes including at least one of a first mode wherein the primary task includes two or more evocative elements presented substantially simultaneously at the user interface, or a second mode wherein the interference includes two or more evocative elements presented substantially simultaneously at the user interface. The one or more processors are also configured to receive data indicative of a first response and a secondary response, at least one of the first response and the secondary response including a measure of a physical action of the individual in response to at least one of the evocative elements, wherein the data includes at least one measure of emotional processing capabilities of the individual under emotional load. The one or more processors analyze the data indicative of the first response and the secondary response to generate a first performance metric including at least one quantified indicator of cognitive abilities of the individual under emotional load, adjust a difficulty of at least one of the primary task and/or the interference based on the generated at least one first performance metric such that the primary task with the interference are rendered at a second difficulty level in a second iteration, and generate a second performance metric representative of cognitive abilities of the individual under emotional load based at least in part on the data indicative of the first response and the second response from the second iteration to provide an indication of a difference in cognitive abilities of the individual.

In yet another aspect, embodiments of the invention relate to a system for enhancing cognitive skills in an individual using evocative elements presented according to one or more integration rules. The system includes one or more processors, and a memory to store processor-executable instructions and communicatively coupled with the one or more processors. Upon execution of the processor-executable instructions by the one or more processors, the one or more processors are configured to generate a user interface, and present via the user interface, at a first difficulty level in a first iteration, a first instance of a primary task in the presence of a secondary task including an interference configured to divert the individual's attention from the first instance of the primary task, requiring a first response from the individual to the first instance of the primary task in the presence of the interference and a secondary response from the individual to the interference. The interference includes a plurality of evocative elements presented according to the one or more integration rules, at least one of the evocative elements being configured as a distractor and at least one of the evocative elements being configured as an interruptor and having either a specified facial expression or a specified non-evocative feature. The one or more integration rules are configured such that the plurality of evocative elements are presented with at least two differing non-evocative features, each non-evocative feature either being correlated with a specific facial expression or not correlated with any facial expressions. The one or more processors are configured to receive data indicative of the first response and the secondary response, at least one of the first response and the secondary response including a measure of the individual's response to the evocative elements, wherein (i) the response includes a physical action of the individual in response to the evocative elements and (ii) the data includes at least one measure of emotional processing capabilities of the individual under emotional load. The one or more processors are configured to analyze the data indicative of the first response and the secondary response to generate a first performance metric including at least one quantified indicator of cognitive abilities of the individual under emotional load, and to adjust a difficulty of at least one or more of the primary task and the interference based on the first performance metric such that the apparatus renders the primary task with the interference at a second difficulty level. The one or more processors are configured to generate a second performance metric representative of cognitive abilities of the individual under emotional load based at least in part on the data indicative of the first response and the second response from the second iteration to provide an indication of a difference in cognitive abilities of the individual.

One or more of the following features may be included. The one or more processors may be further configured to render the first instance of the primary task and the interference to obtain the first and second responses in an iterative manner, with the difficulty level being adjusted between two or more of the iterations. Adjusting the difficulty level may include modifying a time-varying aspect of the first instance of the primary task and/or the interference Modifying the time-varying characteristics of an aspect of the primary task or the interference may include adjusting a temporal length of the rendering of the task or interference at the user interface between two or more sessions of interactions of the individual. The time-varying characteristics may be at least one of a speed of an evocative element, a rate of change of a facial expression, a direction of trajectory of an evocative element, a change of orientation of an evocative element, at least one color of an evocative element, a type of an evocative element, or a size of an evocative element. The change in type of evocative element may be effected using morphing from a first type of evocative element to a second type of evocative element or rendering a blendshape as a proportionate combination of the first type of evocative element and the second type of evocative element.

### BRIEF DESCRIPTION OF DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 shows a block diagram of an example system, according to the principles herein.

FIG. 2 shows a block diagram of an example computing device, according to the principles herein.

FIG. 3A shows an example graphical depiction of a drift-diffusion model for linear belief accumulation, according to the principles herein.

FIG. 3B shows an example graphical depiction of a drift-diffusion model for non-linear belief accumulation, according to the principles herein.

FIG. 4 shows an example plot of the signal and noise based on an example cognitive platform, according to the principles herein.

FIGS. 5A-5D show example user interfaces with instructions to a user that can be presented to an example user interface, according to the principles herein.

FIGS. 6A-6B show examples of the evocative elements and a user interface including instructions for user interaction, according to the principles herein.

FIGS. 7A-7D show examples of the time-varying features of example objects (targets or non-targets) that can be presented to an example user interface, according to the principles herein.

FIGS. 8A-8T show a non-limiting example of the dynamics of tasks and interferences that can be presented at user interfaces, according to the principles herein.

FIGS. 9A-9P show a non-limiting example of the dynamics of tasks and interferences that can be presented at user interfaces, according to the principles herein.

FIGS. 10A-10R show a non-limiting example of the dynamics of tasks and interferences that can be presented according to a mode at user interfaces, according to the principles herein.

FIGS. 11A-11R show a non-limiting example of the dynamics of tasks and interferences that can be presented according to another mode at user interfaces, according to the principles herein.

FIGS. 12A-12E show a non-limiting example of the dynamics of tasks and interferences that can be presented according to an integration rule at user interfaces, according to the principles herein.

FIGS. 13A-13E show a non-limiting example of the dynamics of tasks and interferences that can be presented according to another integration rule at user interfaces, according to the principles herein.

FIGS. 14A-14E show a non-limiting example of the dynamics of tasks and interferences that can be presented according to another integration rule at user interfaces, according to the principles herein.

FIGS. 15A-15E show a non-limiting example of the dynamics of tasks and interferences that can be presented according to another mode at user interfaces, according to the principles herein. In this example, the interference is presented as target/interruptor vs. non-target/distractor evocative elements in a single non-evocative feature (such as but not limited to the color), but with facial expression being varied using primarily the eyes of the evocative element, where the eyes make the expressions (e.g., not by a shape of a mouth), and remainder of rendered creature/face is a blank, dark, or neutral color. In another example, the evocative element may be configured such that makes the mouth makes the expression, or both the eyes and mouth are configured to make the expression. In this example, the color is black, however, it can be any other color such as but not limited to brown, blue, white, or other neutral color.

FIGS. 16A-16C show a non-limiting example of the dynamics of tasks and interferences that can be presented according to differing integration rules at user interfaces, according to the principles herein.

FIGS. 17A-17C show flowchart of example methods, according to the principles herein.

FIG. 18 shows the architecture of an example computer system, according to the principles herein.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

Following below are more detailed descriptions of various concepts related to, and embodiments of, inventive methods, apparatus and systems comprising a cognitive platform configured for using evocative elements rendered in modes (i.e., emotional or affective elements) in computerized tasks (including computerized tasks that appear to a user as platform interactions) that employ one or more interactive user elements to provide cognitive assessment or deliver a cognitive treatment. The example cognitive platform can be associated with a computer-implemented device platform that implements processor-executable instructions (including software programs) to provide an indication of the individual's performance, and/or for cognitive assessment, and/or to deliver a cognitive treatment. In the various examples, the computer-implemented device can be configured as a computer-implemented medical device or other type of computer-implemented device.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

As used herein, the term “includes” means includes but is not limited to, the term “including” means including but not limited to. The term “based on” means based at least in part on.

As used herein, the term “target” refers to a type of stimulus that is specified to an individual (e.g., in instructions) to be the focus for an interaction. A target differs from a non-target in at least one characteristic or feature. Two targets may differ from each other by at least one characteristic or feature, but overall are still instructed to an individual as a target, in an example where the individual is instructed/required to make a choice (e.g., between two different degrees of a facial expression or other characteristic/feature difference, such as but not limited to between a happy face and a happier face or between an angry face and an angrier face). In another non-limiting example, the individual may be instructed to select an evocative element having a specified color and/or a specified facial expression as the target.

As used herein, the term “non-target” refers to a type of stimulus that is not to be the focus for an interaction, whether indicated explicitly or implicitly to the individual (i.e., not specified as the target). For example, the individual may be instructed that an evocative element having any color other than a specified color is the non-target, and/or that an evocative element having any facial expression other than a specified facial expression is the non-target.

In an example implementation described hereinbelow, the individual may be instructed to select an evocative element having a specified color and/or a specified facial expression as the target, while the non-target is: (i) an evocative element having any color other than the specified color, (ii) an evocative element having any facial expression other than the specified facial expression, and (iii) an evocative element having both the specified color and the specified facial expression.

As used herein, the term “task” refers to a goal and/or objective to be accomplished by an individual. Using the example systems, methods, and apparatus described herein, the computerized task is rendered using programmed computerized components, and the individual is instructed (e.g., using a computing device) as to the intended goal or objective from the individual for performing the computerized task. The task may require the individual to provide or withhold a response to a particular stimulus, using at least one component of the computing device (e.g., one or more sensor components of the computing device). The “task” can be configured as a baseline cognitive function that is being measured.

As used herein, the term “interference” refers to a type of stimulus presented to the individual such that it interferes with the individual's performance of a primary task. In any example herein, an interference is a type of task that is presented/rendered in such a manner that it diverts or interferes with an individual's attention in performing another task (including the primary task). In some examples herein, the interference is configured as a secondary task that is presented simultaneously with a primary task, either over a short, discrete time period or over an extended time period (less than the time frame over which the primary task is presented), or over the entire period of time of the primary task. In any example herein, the interference can be presented/rendered continuously, or continually (i.e., repeated in a certain frequency, irregularly, or somewhat randomly). For example, the interference can be presented at the end of the primary task or at discrete, interim periods during presentation of the primary task. The degree of interference can be modulated based on the type, amount, and/or temporal length of presentation of the interference relative to the primary task.

As used herein, the term “stimulus,” refers to a sensory event configured to evoke a specified functional response from an individual. The degree and type of response can be quantified based on the individual's interactions with a measuring component (including using sensor devices or other measuring components). Non-limiting examples of a stimulus include a navigation path (with an individual being instructed to control an avatar or other processor-rendered guide to navigate the path), or a discrete object, whether a target or a non-target, rendered to a user interface (with an individual being instructed to control a computing component to provide input or other indication relative to the discrete object). In any example herein, the task and/or interference includes a stimulus, which can be an evocative element as described hereinbelow.

As used herein, a “trial” includes at least one iteration of rendering of a task and/or interference (either or both with evocative element) and at least one receiving of the individual's response(s) to the task and/or interference (either or both with evocative element). As non-limiting examples, a trial can include at least a portion of a single-tasking task and/or at least a portion of a multi-tasking task. For example, a trial can be a period of time during a navigation task (including a visuo-motor navigation task) in which the individual's performance is assessed, such as but not limited to, assessing whether or the degree of success to which an individual's actions in interacting with the platform result in a guide (including a computerized avatar) navigating along at least a portion of a certain path or in an environment for a time interval (such as but not limited to, fractions of a second, a second, several seconds, or more) and/or causes the guide (including computerized avatar) to cross (or avoid crossing) performance milestones along the path or in the environment. In another example, a trial can be a period of time during a targeting task in which the individual's performance is assessed, such as but not limited to, assessing whether or the degree of success to which an individual's actions in interacting with the platform result in identification/selection of a target versus a non-target (e.g., red object versus yellow object), or discriminates between two different types of targets (a happy face versus a happier face). In these examples, the segment of the individual's performance that is designated as a trial for the navigation task does not need to be co-extensive or aligned with the segment of the individual's performance that is designated as a trial for the targeting task.

In any example herein, an object may be rendered as a depiction of a physical object (including a polygonal or other object), a face (human or non-human), or a caricature, other type of object.

In any of the examples herein, instructions can be provided to the individual to specify how the individual is expected to perform the task and/or interference (either or both with evocative element) in a trial and/or a session. In non-limiting examples, the instructions can inform the individual of the expected performance of a navigation task (e.g., stay on this path, go to these parts of the environment, cross or avoid certain milestone objects in the path or environment), a targeting task (e.g., describe or show the type of object that is the target object versus the non-target object, or describe or show the type of object that is the target object versus the non-target object, or two different types of target object that the individual is expected to choose between (e.g., happy face versus happier face)), and/or describe how the individual's performance is to be scored. In examples, the instructions may be provided visually (e.g., based on a rendered user interface) or via sound. In various examples, the instructions may be provided once prior to the performance two or more trials or sessions, or repeated each time prior to the performance of a trial or a session, or some combination thereof.

While some example systems, methods, and apparatus described herein are based on an individual being instructed/required to decide/select between a target versus a non-target may, in other example implementations, the example systems, methods, and apparatus can be configured such that the individual is instructed/required to decide/choose between two different types of targets (such as but not limited to between two different degrees of a facial expression or other characteristic/feature difference).

In addition, while example systems, methods, and apparatus may be described herein relative to an individual, in other example implementations, the example systems, methods, and apparatus can be configured such that two or more individuals, or members of a group (including a clinical population), perform the tasks and/or interference (either or both with evocative element), either individually or concurrently.

The example platform products and cognitive platforms according to the principles described herein can be applicable to many different types of conditions, such as but not limited to social anxiety, depression, bipolar disorder, major depressive disorder, post-traumatic stress disorder, schizophrenia, autism spectrum disorder, attention deficit hyperactivity disorder, dementia, Parkinson's disease, Huntington's disease, or other neurodegenerative condition, Alzheimer's disease, or multiple sclerosis.

The instant disclosure is directed to computer-implemented devices formed as example platform products configured to implement software or other processor-executable instructions for the purpose of measuring data indicative of a user's performance at one or more tasks, to provide a user performance metric. The performance metric can be used to derive an assessment of a user's cognitive abilities under emotional load and/or to measure a user's response to a cognitive treatment, and/or to provide data or other quantitative indicia of a user's mood or cognitive or affective bias. As used herein, indicia of cognitive or affective bias include data indicating a user's preference for a negative emotion, perspective, or outcome as compared to a positive emotion, perspective, or outcome.

In a non-limiting example implementation, the example platform product herein may be formed as, be based on, or be integrated with, an AKILI™ platform product (also referred to herein as an “APP”) by Akili Interactive Labs, Inc., Boston, Mass.

As described in greater detail below, the computing device can include an application (an “App program”) to perform such functionalities as analyzing the data. For example, the data from the at least one sensor component can be analyzed as described herein by a processor executing the App program on an example computing device to receive (including to measure) substantially simultaneously two or more of: (i) the response from the individual to a task, (i) a secondary response of the individual to an interference, and (iii) a response of the individual to at least one evocative element. As another example, the data from the at least one sensor component can be analyzed as described herein by a processor executing the App program on an example computing device to analyze the data indicative of the first response and the response of the individual to the at least one evocative element to compute at least one performance metric comprising at least one quantified indicator of cognitive abilities.

An example system according to the principles herein provides for generating a quantifier of cognitive skills in an individual (including using a machine learning classifier) and/or enhancing cognitive skills in an individual. In an example implementation, the example system employs an App program running on a mobile communication device or other hand-held devices. Non-limiting examples of such mobile communication devices or hand-held device include a smartphone, such as but not limited to an iPhone®, a BlackBerry®, or an Android-based smartphone, a tablet, a slate, an electronic-reader (e-reader), a digital assistant, or other electronic reader or hand-held, portable, or wearable computing device, or any other equivalent device, an Xbox®, a Wii®, a Playstation®, or other gaming system, or other computing system that can be used to render game-like elements. In some example implementations, the example system can include a head-mounted device, such as smart eyeglasses with built-in displays, a smart goggle with built-in displays, or a smart helmet with built-in displays, and the user can hold a controller or an input device having one or more sensors in which the controller or the input device communicates wirelessly with the head-mounted device. In some example implementations, the computing system may be stationary, such as a desktop computing system that includes a main computer and a desktop display (or a projector display), in which the user provides inputs to the App program using a keyboard, a computer mouse, a joystick, handheld consoles, wristbands, or other wearable devices having sensors that communicate with the main computer using wired or wireless communication. In other examples herein, the example system may be a virtual reality system, an augmented reality system, or a mixed reality system. In examples herein, the sensors can be configured to measure movements of the user's hands, feet, and/or any other part of the body. In some example implementations, the example system can be formed as a virtual reality (VR) system (a simulated environment including as an immersive, interactive 3-D experience for a user), an augmented reality (AR) system (including a live direct or indirect view of a physical, real-world environment whose elements are augmented by computer-generated sensory input such as but not limited to sound, video, graphics and/or GPS data), or a mixed reality (MR) system (also referred to as a hybrid reality which merges the real and virtual worlds to produce new environments and visualizations where physical and digital objects co-exist and interact substantially in real time).

As used herein, the term “cData” refers to data collected from measures of an interaction of a user with a computer-implemented device formed as a platform product.

As used herein, the term “computerized stimuli or interaction” or “CSI” refers to a computerized element that is presented to a user to facilitate the user's interaction with a stimulus or other interaction. As non-limiting examples, the computing device can be configured to present auditory stimulus (presented, e.g., as an auditory evocative element or an element of a computerized auditory task) or initiate other auditory-based interaction with the user, and/or to present vibrational stimuli (presented, e.g., as a vibrational evocative element or an element of a computerized vibrational task) or initiate other vibrational-based interaction with the user, and/or to present tactile stimuli (presented, e.g., as a tactile evocative element or an element of a computerized tactile task) or initiate other tactile-based interaction with the user, and/or to present visual stimuli or initiate other visual-based interaction with the user.

In an example where the computing device is configured to present visual CSI, the CSI can be rendered at at least one user interface to be presented to a user. In some examples, the at least one user interface is configured for measuring responses as the user interacts with CSI computerized element rendered at the at least one user interface. In a non-limiting example, the user interface can be configured such that the CSI computerized element(s) are active, and may require at least one response from a user, such that the user interface is configured to measure data indicative of the type or degree of interaction of the user with the platform product. In another example, the user interface can be configured such that the CSI computerized element(s) are a passive and are presented to the user using the at least one user interface but may not require a response from the user. In this example, the at least one user interface can be configured to exclude the recorded response of an interaction of the user, to apply a weighting factor to the data indicative of the response (e.g., to weight the response to lower or higher values), or to measure data indicative of the response of the user with the platform product as a measure of a misdirected response of the user (e.g., to issue a notification or other feedback to the user of the misdirected response).

In an example, the platform product can be configured as a processor-implemented system, method or apparatus that includes a display component, an input device, and at least one processing unit. In an example, the at least one processing unit can be programmed to render at least one user interface, for display at the display component, to present the computerized stimuli or interaction (CSI) or other interactive elements to the user for interaction. In other examples, the at least one processing unit can be programmed to cause an actuating component of the platform product to effect auditory, tactile, or vibrational computerized elements (including CSIs) to effect the stimulus or other interaction with the user. The at least one processing unit can be programmed to cause a component of the program product to receive data indicative of at least one user response based on the user interaction with the CSI or other interactive element (such as but not limited to cData), including responses provided using the input device. In an example where at least one user interface is rendered to present the computerized stimuli or interaction (CSI) or other interactive elements to the user, the at least one processing unit can be programmed to cause user interface to receive the data indicative of at least one user response. The at least one processing unit also can be programmed to: analyze the differences in the individual's performance based on determining the differences between the user's responses, and/or adjust the difficulty level of the computerized stimuli or interaction (CSI) or other interactive elements based on the individual's performance determined in the analysis, and/or provide an output or other feedback from the platform product indicative of the individual's performance, and/or cognitive assessment, and/or response to cognitive treatment. In some examples, the results of the analysis may be used to modify the difficulty level or other property of the computerized stimuli or interaction (CSI) or other interactive elements.

In a non-limiting example, the computerized element includes at least one task rendered at a user interface as a visual task or presented as an auditory, tactile, or vibrational task. Each task can be rendered as interactive mechanics that are designed to elicit a response from a user after the user is exposed to stimuli for the purpose of cData collection.

In a non-limited example of a computerized auditory task, the individual may be required to follow a certain computer-rendered path or navigate other environment based on auditory cues emitted to the individual. The processing unit may be configured to cause an auditory component to emit the auditory cues (e.g., sounds or human voices) to provide the individual with performance progress milestones to maintain or modify the path of a computerized avatar in the computer environment, and/or to indicate to the individual their degree of success in performing the physical actions measured by the sensors of the computing device to cause the computerized avatar to maintain the expected course or path.

In a non-limited example of a computerized vibrational task, the individual may be required to follow a certain computer-rendered path or navigate other environment based on vibrational cues emitted to the individual. The processing unit may be configured to control an actuating component to vibrate (including causing a component of the computing device to vibrate) to provide the individual with the performance progress milestones to maintain or modify the path of a computerized avatar in the computer environment, and/or to indicate to the individual their degree of success in performing the physical actions measured by the sensors of the computing device to cause the computerized avatar to maintain the expected course or path.

In a non-limited example of a computerized auditory task, the individual may be required to interact with one or more sensations perceived through the sense of touch. In a non-limiting example, an evocative element may be controlled using a processing unit to actuate an actuating component to present differing types of tactile stimuli (e.g., sensation of touch, textured surfaces or temperatures) for interaction with an individual. For example, an individual with an autism spectrum disorder (ASD) may be sensitive to (including having an aversion to) certain tactile sensory sensations (including being touched as they dress or groom themselves); individuals with Alzheimer's disease and other dementias may benefit through the sense of touch or other tactile sensation. An example tactile task may engage a tactile-sensitive individual in physical actions that causes them to interact with textures and touch sensations.

In a non-limiting example, the computerized element includes at least one platform interaction (gameplay) element of the platform rendered at a user interface, or as auditory, tactile, or vibrational element of a program product. Each platform interaction (gameplay) element of the platform product can include interactive mechanics (including in the form of videogame-like mechanics) or visual (or cosmetic) features that may or may not be targets for cData collection.

As used herein, the term “gameplay” encompasses a user interaction (including other user experience) with aspects of the platform product.

In a non-limiting example, the computerized element includes at least one element to indicate positive feedback to a user. Each element can include an auditory signal and/or a visual signal emitted to the user that indicates success at a task or other platform interaction element, i.e., that the user responses at the platform product has exceeded a threshold success measure on a task or platform interaction (gameplay) element.

In a non-limiting example, the computerized element includes at least one element to indicate negative feedback to a user. Each element can include an auditory signal and/or a visual signal emitted to the user that indicates failure at a task or platform interaction (gameplay) element, i.e., that the user responses at the platform product has not met a threshold success measure on a task or platform interaction element.

In a non-limiting example, the computerized element includes at least one element for messaging, i.e., a communication to the user that is different from positive feedback or negative feedback.

In a non-limiting example, the computerized element includes at least one element for indicating a reward. A reward computer element can be a computer generated feature that is delivered to a user to promote user satisfaction with the CSIs and as a result, increase positive user interaction (and hence enjoyment of the user experience).

In a non-limiting example, the cognitive platform can be configured to render at least one evocative element (i.e., an emotional/affective element, “EAE”). As used herein, an “evocative element” is a computerized element that is configured to evoke from the individual an emotional response (i.e., a response based on the individual's cognitive and/or neurologic processing of emotion/affect/mood or parasympathetic arousal) and/or an affective response (i.e., a response based on the individual's preference for a negative emotion, perspective, or outcome as compared to a positive emotion, perspective, or outcome).

In the various examples herein, the evocative elements rendered in modes (i.e., emotional elements and/or affective elements) can be rendered as CSIs including images (including images of faces), sounds (including voices), or words can represent or correlate with expressions of a specific emotion or combination of emotions to a user or to evoke cognitive and biological states reflecting a specific emotion or combination of emotions in a user. The example evocative elements are configured to evoke a response from an individual. In an example, the evocative element can be rendered faces (including faces of human or non-human animals, or animated creatures) having differing expressions of differing valence, such as but not limited to expressions of negative valence (e.g., angry or disgusted expressions), expressions of positive valence (e.g., happy expressions), or neutral expressions. In an example, the evocative element can be rendered as emotional sounds or voices which is effected using a computing device, e.g., using an actuating, audio, microphone, or other component. In other examples, the evocative elements rendered in modes can be specifically customized to an individual. As non-limiting examples, the evocative element can be rendered as a scene related to an individual's phobia or post-traumatic stress disorder (PTSD) (e.g., heights for those fearful of heights), aversively conditioned stimuli, feared or stressful objects in people with specific phobias (e.g., snakes, spiders, or other feared object or situation), or threat words. In other examples, the evocative elements rendered in modes can be rendered based on the processing unit actuating a component to generate an auditory, tactile, or vibrational computerized element.

In examples, the evocative elements rendered in modes can be rendered as example words represent or correlate with expressions of a specific emotion or combination of emotions. For example, the words may be neutral, or words that evoke threat or fear, or contentment, or other types of words. As a non-limiting example, the words may be associated with a threat (threat words) such as “tumor”, “torture”, “crash”, or “horror”, or may be neutral words, such as “table” or “picture”, or may be positive words, such as “happy”, “content”, or “smile”.

In a non-limiting example, the cognitive platform can be configured to render multi-task interactive elements. In some examples, the multi-task interactive elements are referred to as multi-task gameplay (MTG). The multi-task interactive elements include interactive mechanics configured to engage the user in multiple temporally-overlapping tasks, i.e., tasks that may require multiple, substantially simultaneous responses from a user.

In any example herein, the multi-tasking tasks can include any combination of two or more tasks. The multi-task interactive elements of an implementation include interactive mechanics configured to engage the individual in multiple temporally-overlapping tasks, i.e., tasks that may require multiple, substantially simultaneous responses from an individual. In non-limiting examples herein, in an individual's performance of at least a portion of a multi-tasking task, the system, method, and apparatus are configured to measure data indicative of the individual's multiple responses in real-time, and also to measure a first response from the individual to a task (as a primary task) substantially simultaneously with measuring a second response from the individual to an interference (as a secondary task).

In an example implementation involving multi-tasking tasks, the computer device is configured (such as using at least one specially-programmed processing unit) to cause the cognitive platform to present to a user two or more different types of tasks, such as but not limited to, target discrimination and/or navigation and/or facial expression recognition or object recognition tasks, during a short time frame (including in real-time and/or substantially simultaneously). The computer device is also configured (such as using at least one specially-programmed processing unit) to collect data indicative of the type of user response received for the multi-tasking tasks, within the short time frame (including in real-time and/or substantially simultaneously). In these examples, the two or more different types of tasks can be presented to the individual within the short time frame (including in real-time and/or substantially simultaneously), and the computing device can be configured to receive data indicative of the user response(s) relative to the two or more different types of tasks within the short time frame (including in real-time and/or substantially simultaneously).

Based on the type of computerized task presented to an individual using the cognitive platform, the types of response(s) expected as a result of the individual interacting with the cognitive platform to perform the task(s), and types of data expected to be received (including being measured) using the cognitive platform, depends on the type of the task(s). For a target discrimination task, the cognitive platform may require a temporally-specific and/or a position-specific response from an individual, including to select between a target and a non-target (e.g., in a GO/NO-GO task) or to select between two differing types of targets, e.g., in a two-alternative forced choice (2AFC) task (including choosing between two differing degrees of a facial expression or other characteristic/feature difference). For a navigation task, the cognitive platform may require a position-specific and/or a motion-specific response from the user. For a facial expression recognition or object recognition task, the cognitive platform may require temporally-specific and/or position-specific responses from the user. In non-limiting examples, the user response to tasks, such as but not limited to targeting and/or navigation and/or facial expression recognition or object recognition task(s), can be recorded using an input device of the cognitive platform. Non-limiting examples of such input devices can include a device for capturing a touch, swipe or other gesture relative to a user interface, an audio capture device (e.g., a microphone input), or an image capture device (such as but not limited to a touch-screen or other pressure-sensitive or touch-sensitive surface, or a camera), including any form of user interface configured for recording a user interaction. In other non-limiting examples, the user response recorded using the cognitive platform for tasks, such as but not limited to targeting and/or navigation and/or facial expression recognition or object recognition task(s), can include user actions that cause changes in a position, orientation, or movement of a computing device including the cognitive platform. Such changes in a position, orientation, or movement of a computing device can be recorded using an input device disposed in or otherwise coupled to the computing device, such as but not limited to a sensor. Non-limiting examples of sensors include a motion sensor, position sensor, and/or an image capture device (such as but not limited to a camera).

In the example herein, “substantially simultaneously” means tasks are rendered, or response measurements are performed, within less than about 5 milliseconds of each other, or within about 10 milliseconds, about 20 milliseconds, about 50 milliseconds, about 75 milliseconds, about 100 milliseconds, or about 150 milliseconds or less, about 200 milliseconds or less, about 250 milliseconds or less, of each other. In any example herein, “substantially simultaneously” is a period of time less than the average human reaction time. In another example, two tasks may be substantially simultaneous if the individual switches between the two tasks within a pre-set amount of time. The set amount of time for switching considered “substantially simultaneously” can be about 1 tenth of a second, 1 second, about 5 seconds, about 10 seconds, about 30 seconds, or greater.

In some examples, the short time frame can be of any time interval at a resolution of up to about 1.0 millisecond or greater. The time intervals can be, but are not limited to, durations of time of any division of a periodicity of about 2.0 milliseconds or greater, up to any reasonable end time. The time intervals can be, but are not limited to, about 3.0 millisecond, about 5.0 millisecond, about 10 milliseconds, about 25 milliseconds, about 40 milliseconds, about 50 milliseconds, about 60 milliseconds, about 70 milliseconds, about 100 milliseconds, or greater. In other examples, the short time frame can be, but is not limited to, fractions of a second, about a second, between about 1.0 and about 2.0 seconds, or up to about 2.0 seconds, or more.

In any example herein, the cognitive platform can be configured to collect data indicative of a reaction time of a user's response relative to the time of presentation of the tasks (including an interference with a task). For example, the computing device can be configured to cause the platform product or cognitive platform to provide smaller or larger reaction time window for a user to provide a response to the tasks as an example way of adjusting the difficulty level.

In a non-limiting example, the cognitive platform can be configured to render single-task interactive elements. In some examples, the single-task interactive elements are referred to as single-task gameplay (STG). The single-task interactive elements include interactive mechanics configured to engage the user in a single task in a given time interval.

According to the principles herein, the term “cognition” refers to the mental action or process of acquiring knowledge and understanding through thought, experience, and the senses. This includes, but is not limited to, psychological concepts/domains such as, executive function, memory, perception, attention, emotion, motor control, and interference processing. An example computer-implemented device according to the principles herein can be configured to collect data indicative of user interaction with a platform product, and to compute metrics that quantify user performance. The quantifiers of user performance can be used to provide measures of cognition (for cognitive assessment) or to provide measures of status or progress of a cognitive treatment.

According to the principles herein, the term “treatment” refers to any manipulation of CSI in a platform product (including in the form of an APP) that results in a measurable improvement of the abilities of a user, such as but not limited to improvements related to cognition, a user's mood or level of cognitive or affective bias. The degree or level of improvement can be quantified based on user performance measures as describe herein.

According to the principles herein, the term “session” refers to a discrete time period, with a clear start and finish, during which a user interacts with a platform product to receive assessment or treatment from the platform product (including in the form of an APP). In examples herein, a session can refer to at least one trial or can include at least one trial and at least one other type of measurement and/or other user interaction. As a non-limiting example, a session can include at least one trial and one or more of a measurement using a physiological or monitoring component and/or a cognitive testing component. As another non-limiting example, a session can include at least one trial and receipt of data indicative of one or more measures of an individual's condition, including physiological condition and/or cognitive condition.

According to the principles herein, the term “assessment” refers to at least one session of user interaction with CSIs or other feature or element of a platform product. The data collected from one or more assessments performed by a user using a platform product (including in the form of an APP) can be used as to derive measures or other quantifiers of cognition, or other aspects of a user's abilities.

According to the principles herein, the term “cognitive load” refers to the amount of mental resources that a user may need to expend to complete a task. This term also can be used to refer to the challenge or difficulty level of a task or gameplay.

According to the principles herein, the term “emotional load” refers to cognitive load that is specifically associated with processing emotional information or regulating emotions or with affective bias in an individual's preference for a negative emotion, perspective, or outcome as compared to a positive emotion, perspective, or outcome.

According to the principles herein, the term “ego depletion” refers to a state reached by a user after a period of effortful exertion of self-control, characterized by diminished capacity to exert further self-control. The state of ego-depletion may be measured based on data collected for a user's responses to the interactive elements rendered at user interfaces, or as auditory, tactile, or vibrational elements, of a platform product described hereinabove.

According to the principles herein, the term “emotional processing” refers to a component of cognition specific to cognitive and/or neurologic processing of emotion/affect/mood or parasympathetic arousal. The degree of emotional processing may be measured based on data collected for a user's responses to the interactive elements rendered at user interfaces, or as auditory, tactile, or vibrational elements, of a platform product described hereinabove.

An example system, method, and apparatus according to the principles herein includes a platform product (including using an APP) that uses a cognitive platform configured to render at least one evocative element (EAE), to add emotional processing as an overt component for tasks in MTG or STG. In one example, the evocative element (EAE) is used in the tasks configured to assess cognition or improve cognition related to emotions, and the data (including cData) collected as a measure of user interaction with the rendered evocative element (EAE) in the platform product is used to determine the measures of the assessment of cognition or the improvement to measures of cognition after a treatment configured for interaction using the user interface, or as auditory, tactile, or vibrational elements, of the platform product. The evocative element (EAE) can be configured to collect data to measure the impact of emotions on non-emotional cognition, such as by causing the user interface to render spatial tasks for the user to perform under emotional load, and/or to collect data to measure the impact of non-emotional cognition on emotions, such as by causing the user interface to render features that employ measures of executive function to regulate emotions. In one example implementation, the user interface can be configured to render tasks for identifying the emotion indicated by the CSI (based on measurement data), maintaining that identification in working memory, and comparing it with the measures of emotion indicated by subsequent CSI, while under cognitive load due to MTG.

In one example, the user interface may be configured to present to a user a program platform based on a cognitive platform based on interference processing. In an example system, method and apparatus that implements interference processing, the at least one processing unit is programmed to render at least one first user interface, or auditory, tactile, or vibrational signal, to present a first task that requires a first type of response from a user, and to render at least one second user interface, or auditory, tactile, or vibrational signal, to present a first interference with the first task, requiring a second type of response from the user to the first task in the presence of the first interference. In a non-limiting example, the second type of response can include the first type of response to the first task and a secondary response to the first interference. In another non-limiting example, the second type of response may not include, and be quite different from, the first type of response. The at least one processing unit is also programmed to receive data indicative of the first type of response and the second type of response based on the user interaction with the platform product (such as but not limited to cData), such as but not limited to by rendering the at least one user interface to receive the data. The at least one processing unit also can be programmed to: analyze the differences in the individual's performance based on determining the differences between the measures of the user's first type and second type of responses, and/or adjust the difficulty level of the first task and/or the first interference based on the individual's performance determined in the analysis, and/or provide an output or other feedback from the platform product that can be indicative of the individual's performance, and/or cognitive assessment, and/or response to cognitive treatment, and/or assessed measures of cognition. As a non-limiting example, the cognitive platform based on interference processing can be the Project:EVO™ platform by Akili Interactive Labs, Inc., Boston, Mass.

In an example system, method and apparatus according to the principles herein that is based on interference processing, the user interface is configured such that, as a component of the interference processing, one of the discriminating features of the targeting task that the user responds to is a feature in the platform that displays an emotion, similar to the way that shape, color, and/or position may be used in an interference element in interference processing.

In another example system, method and apparatus according to the principles herein that is based on interference processing, a platform product may include a working-memory task such as cognitive tasks that employs evocative element (EAE), where the affective content is either a basis for matching or a distractive element as part of the user interaction, within a MTG or a STG.

An example system, method, and apparatus according to the principles herein includes a platform product (including using an APP) that uses a cognitive platform configured to render at least one integrating evocative element (EAE) in a MTG or a STG, where the user interface is configured to not explicitly call attention to the evocative element (EAE). The user interface of the platform product may be configured to render evocative element (EAE) for the purpose of assessing or adjusting emotional biases in attention, interpretation, or memory, and to collected data indicative of the user interaction with the platform product.

An example system, method, and apparatus according to the principles herein includes a platform product (including using an APP) that uses a cognitive platform configured to render at least one evocative element (EAE) that reinforces positive or negative feedback provided within the one or more tasks.

An example system, method, and apparatus according to the principles herein includes a platform product (including using an APP) that uses a cognitive platform configured to render at least one evocative element (EAE) that introduces fixed or adjustable levels of emotional load to the user interaction (including to gameplay). This could be used for the purposes of modulating the difficulty of a MTG or a STG. This includes using evocative element(s) (EAE) that conflicts with the positive feedback or negative feedback provided within the one or more tasks, or using evocative element(s) (EAE) to induce ego depletion to impact the user's cognitive control capabilities.

An example system, method, and apparatus according to the principles herein includes a platform product (including using an APP) that uses a cognitive platform configured to render and integrate at least one simultaneous conflicting evocative element(s) (EAE) into different tasks during a MTG. This could be used for the purpose of assessing or improving measures of cognition related to the user interaction with the platform product indicating the user's handling of conflicting emotional information.

An example system, method, and apparatus according to the principles herein includes a platform product (including using an APP) that uses video or audio sensors to detect the performance of physical or vocal actions by the user, as a means of response to CSI within a task. These actions may be representations of emotions, such as facial or vocal expressions, or words.

An example system, method, and apparatus according to the principles herein includes a platform product (including using an APP) that uses a cognitive platform configured to render at least one evocative element (EAE) as part of an emotional regulation strategy to enable better user engagement with the platform product when the analysis of the collected date indicates that the user is in a non-optimal emotional state. For example, if the data analysis of the performance measures of the platform product determines that the user is frustrated and unable to properly engage in treatment or assessment, the platform product could be configured to introduce some sort of break in the normal interaction sequence that employs evocative elements rendered in modes (EAEs) until after a time interval that the user is deemed ready to engage sufficiently again. This can be a fixed interval of time or an interval of time computed based on the user's previous performance data.

An example system, method, and apparatus according to the principles herein includes a platform product (including using an APP) that uses a cognitive platform configured to render at least one evocative element (EAE) in the interaction sequence, measure user responses, and adjust the CSI accordingly. These measurements may be compared with the user responses to interaction sequences in the platform that do not present evocative elements (EAEs), in order to determine measures of the user's emotional reactivity. This measurement, with or without comparison to measurements made during interaction sequences that do not present evocative elements (EAEs), may be for the purpose of assessing the user's emotional state. The CSI adjustments might be initiating an emotional regulation strategy to enable better engagement with the platform product or initiating certain interactive elements, such as but not limited to tasks or rewards, only under certain emotional conditions. The user response measurement may employ use of inputs such as touchscreens, keyboards, or accelerometers, or passive external sensors such as video cameras, microphones, eye-tracking software/devices, bio-sensors, and/or neural recording (e.g., electroencephalogram), and may include responses that are not directly related to interactions with the platform product, as well as responses based on user interactions with the platform product. The platform product can present measures of a user's emotional state that include a measure of specific moods and/or a measure of general state of ego depletion that impacts emotional reactivity.

An example system, method, and apparatus according to the principles herein includes a platform product (including using an APP) that uses a cognitive platform configured to render at least one evocative element (EAE) to suggest possible appropriate task responses. This may be used to evaluate the user's ability to discern emotional cues, or to choose appropriate emotional responses.

An example system, method, and apparatus according to the principles herein includes a platform product (including using an APP) that uses a cognitive platform configured to render at least one evocative element (EAE) in time-limited tasks, where the time limits may be modulated. This may be for the purposes of measuring user responses via different cognitive processes, such as top-down conscious control vs. bottom-up reflexive response.

An example system, method, and apparatus according to the principles herein includes a platform product (including using an APP) that uses a cognitive platform configured to render at least one evocative element (EAE) with levels of valence determined based on previous user responses to evocative element (EAE) at one or more level of valence. This may apply an adaptive algorithm to progressively adjust the level of valence to achieve specific goals, such as creating a psychometric curve of expected user performance on a task across stimulus or difficulty levels, or determining the specific level at which a user's task performance would meet a specific criterion like 50% accuracy in a Go/No-Go task.

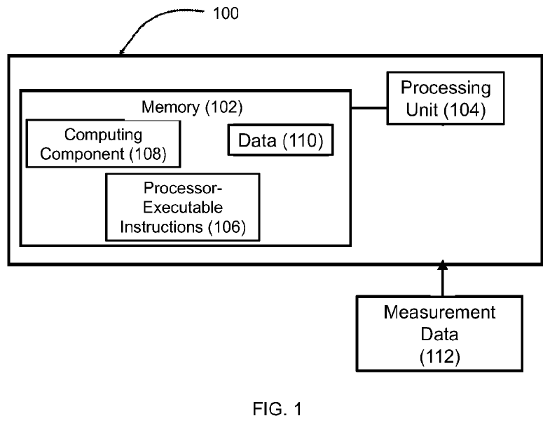

As described hereinabove, the example systems, methods, and apparatus according to the principles herein can be implemented, using at least one processing unit of a programmed computing device, to provide the cognitive platform. FIG. 1 shows an example apparatus 100 according to the principles herein that can be used to implement the cognitive platform described hereinabove herein. The example apparatus 100 includes at least one memory 102 and at least one processing unit 104. The at least one processing unit 104 is communicatively coupled to the at least one memory 102.

Example memory 102 can include, but is not limited to, hardware memory, non-transitory tangible media, magnetic storage disks, optical disks, flash drives, computational device memory, random access memory, such as but not limited to DRAM, SRAM, EDO RAM, any other type of memory, or combinations thereof. Example processing unit 104 can include, but is not limited to, a microchip, a processor, a microprocessor, a special purpose processor, an application specific integrated circuit, a microcontroller, a field programmable gate array, any other suitable processor, or combinations thereof.

The at least one memory 102 is configured to store processor-executable instructions 106 and a computing component 108. In a non-limiting example, the computing component 108 can be used to receive (including to measure) substantially simultaneously two or more of: (i) the response from the individual to a task, (i) a secondary response of the individual to an interference, and (iii) a response of the individual to at least one evocative element. In another non-limiting example, the computing component 108 can be used to analyze the data from the at least one sensor component as described herein and/or to analyze the data indicative of the first response and the response of the individual to the at least one evocative element to compute at least one performance metric comprising at least one quantified indicator of cognitive abilities. In another non-limiting example, the computing component 108 can be used to compute signal detection metrics in computer-implemented adaptive response-deadline procedures. As shown in FIG. 1, the memory 102 also can be used to store data 110, such as but not limited to the measurement data 112. In various examples, the measurement data 112 can include physiological measurement data (including data collected based on one or more measurements) of an individual received from a physiological component (not shown) and/or data indicative of the response of an individual to a task and/or an interference rendered at a user interface of the apparatus 100 (as described in greater detail below), or using an auditory, tactile, or vibrational signal from an actuating component of the apparatus 100, and/or data indicative of one or more of an amount, concentration, or dose titration, or other treatment regimen of a drug, pharmaceutical agent, biologic, or other medication being or to be administered to an individual.

In a non-limiting example, the at least one processing unit 104 executes the processor-executable instructions 106 stored in the memory 102 at least to measure substantially simultaneously two or more of: (i) the response from the individual to a task, (i) a secondary response of the individual to an interference, and (iii) a response of the individual to at least one evocative element. The at least one processing unit 104 also executes the processor-executable instructions 106 stored in the memory 102 at least to analyze the data collected using a measurement component (including the data indicative of the first response and the response of the individual to the at least one evocative element) to compute at least one performance metric comprising at least one quantified indicator of cognitive abilities using the computing component 108. The at least one processing unit 104 also may be programmed to execute processor-executable instructions 106 to control a transmission unit to transmit values indicative of the computed signal detection metrics and/or controls the memory 102 to store values indicative of the signal detection metrics.

In a non-limiting example, the at least one processing unit 104 also executes processor-executable instructions 106 to control a transmission unit to transmit values indicative of the computed performance metric and/or controls the memory 102 to store values indicative of the computed performance metric.

In another non-limiting example, the at least one processing unit 104 executes the processor-executable instructions 106 stored in the memory 102 at least to apply signal detection metrics in computer-implemented adaptive response-deadline procedures.

In any example herein, the user interface may be a graphical user interface.

In another non-limiting example, the measurement data 112 can be collected from measurements using one or more physiological or monitoring components and/or cognitive testing components. In any example herein, the one or more physiological components are configured for performing physiological measurements. The physiological measurements provide quantitative measurement data of physiological parameters and/or data that can be used for visualization of physiological structure and/or functions.

In any example herein, the measurement data 112 can include reaction time, response variance, correct hits, omission errors, number of false alarms (such as but not limited to a response to a non-target), learning rate, spatial deviance, subjective ratings, and/or performance threshold, or data from an analysis, including percent accuracy, hits, and/or misses in the latest completed trial or session. Other non-limiting examples of measurement data 112 include response time, task completion time, number of tasks completed in a set amount of time, preparation time for task, accuracy of responses, accuracy of responses under set conditions (e.g., stimulus difficulty or magnitude level and association of multiple stimuli), number of responses a participant can register in a set time limit number of responses a participant can make with no time limit, number of attempts at a task needed to complete a task, movement stability, accelerometer and gyroscope data, and/or self-rating.

In any example herein, the one or more physiological components can include any means of measuring physical characteristics of the body and nervous system, including electrical activity, heart rate, blood flow, and oxygenation levels, to provide the measurement data 112. This can include camera-based heart rate detection, measurement of galvanic skin response, blood pressure measurement, electroencephalogram, electrocardiogram, magnetic resonance imaging, near-infrared spectroscopy, and/or pupil dilation measures, to provide the measurement data 112. The one or more physiological components can include one or more sensors for measuring parameter values of the physical characteristics of the body and nervous system, and one or more signal processors for processing signals detected by the one or more sensors.

Other examples of physiological measurements to provide measurement data 112 include, but are not limited to, the measurement of body temperature, heart or other cardiac-related functioning using an electrocardiograph (ECG), electrical activity using an electroencephalogram (EEG), event-related potentials (ERPs), functional magnetic resonance imaging (fMRI), blood pressure, electrical potential at a portion of the skin, galvanic skin response (GSR), magneto-encephalogram (MEG), eye-tracking device or other optical detection device including processing units programmed to determine degree of pupillary dilation, functional near-infrared spectroscopy (fNIRS), and/or a positron emission tomography (PET) scanner. An EEG-fMRI or MEG-fMRI measurement allows for simultaneous acquisition of electrophysiology (EEG/MEG) data, hemodynamic (fMRI) data, heart rate, skin conductance, reaction time, and accelerometer measurements.

The example apparatus of FIG. 1 can be configured as a computing device for performing any of the example methods described herein. The computing device can include an App program for performing some of the functionality of the example methods described herein.

In any example herein, the example apparatus can be configured to communicate with one or more of a cognitive monitoring component, a disease monitoring component, and a physiological measurement component, to provide for biofeedback and/or neurofeedback of data to the computing device, for adjusting a type or a difficulty level of one or more of the task, the interference, and the evocative element, to achieve the desired performance level of the individual. As a non-limiting example, the biofeedback can be based on physiological measurements of the individual as they interact with the apparatus, to modify the type or a difficulty level of one or more of the task, the interference, and the evocative element based on the measurement data indicating, e.g., the individual's attention, mood, or emotional state. As a non-limiting example, the neurofeedback can be based on measurement and monitoring of the individual using a cognitive and/or a disease monitoring component as the individual interacts with the apparatus, to modify the type or a difficulty level of one or more of the task, the interference, and the evocative element based on the measurement data indicating, e.g., the individual's cognitive state, disease state (including based on data from monitoring systems or behaviors related to the disease state).

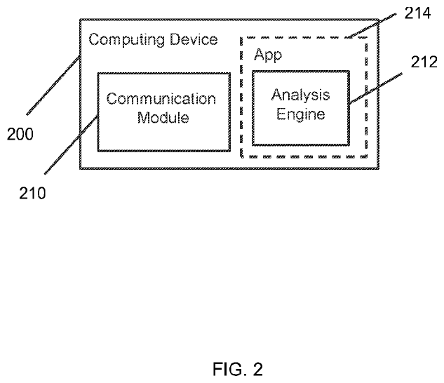

FIG. 2 shows another example apparatus according to the principles herein, configured as a computing device 200 that can be used to implement the cognitive platform according to the principles herein. The example computing device 200 can include a communication module 210 and an analysis engine 212. The communication module 210 can be implemented to receive data indicative of at least one response of an individual to the task in the absence of an interference, and/or at least one response of an individual to the task that is being rendered in the presence of the interference. In an example, the communication module 210 can be implemented to receive substantially simultaneously two or more of: (i) the response from the individual to a task, (ii) a secondary response of the individual to an interference, and (iii) a response of the individual to at least one evocative element. The analysis engine 212 can be implemented to analyze the data from the at least one sensor component as described herein and/or to analyze the data indicative of the first response and the response of the individual to the at least one evocative element to compute at least one performance metric comprising at least one quantified indicator of cognitive abilities. In another example, the analysis engine 212 can be implemented to analyze data to generate a response profile, decision boundary metric (such as but not limited to response criteria), a classifier, and/or other metrics and analyses described herein. As shown in the example of FIG. 2, the computing device 200 can include processor-executable instructions such that a processor unit can execute an application program (App 214) that a user can implement to initiate the analysis engine 212. In an example, the processor-executable instructions can include software, firmware, or other instructions.

The example communication module 210 can be configured to implement any wired and/or wireless communication interface by which information may be exchanged between the computing device 200 and another computing device or computing system. Non-limiting examples of wired communication interfaces include, but are not limited to, USB ports, RS232 connectors, RJ45 connectors, and Ethernet connectors, and any appropriate circuitry associated therewith. Non-limiting examples of wireless communication interfaces may include, but are not limited to, interfaces implementing Bluetooth® technology, Wi-Fi, Wi-Max, IEEE 802.11 technology, radio frequency (RF) communications, Infrared Data Association (IrDA) compatible protocols, Local Area Networks (LAN), Wide Area Networks (WAN), and Shared Wireless Access Protocol (SWAP).

In an example implementation, the example computing device 200 includes at least one other component that is configured to transmit a signal from the apparatus to a second computing device. For example, the at least one component can include a transmitter or a transceiver configured to transmit a signal including data indicative of a measurement by at least one sensor component to the second computing device.

In any example herein, the App 214 on the computing device 200 can include processor-executable instructions such that a processor unit of the computing device implements an analysis engine to analyze data indicative of the individual's response to the rendered tasks and/or interference (either or both with evocative element) and the response of the individual to the at least one evocative element to compute at least one performance metric comprising at least one quantified indicator of cognitive abilities. In another example, the App 214 on the computing device 200 can include processor-executable instructions such that a processor unit of the computing device implements an analysis engine to analyze the data indicative of the individual's response to the rendered tasks and/or interference (either or both with evocative element) and the response of the individual to the at least one evocative element to provide a classifier based on the computed values of the performance metric, to generate a classifier output indicative of a measure of cognition, a mood, a level of cognitive bias, or an affective bias of the individual. In some examples, the App 214 can include processor-executable instructions such that the processing unit of the computing device implements the analysis engine to provide a classifier as to response profile, decision boundary metric (such as but not limited to response criteria), a classifier, and other metrics and analyses described herein. In some example, the App 214 can include processor-executable instructions to provide one or more of: (i) a classifier output indicative of the cognitive capabilities of the individual under emotional load, (ii) a likelihood of the individual experiencing an adverse event in response to administration of the pharmaceutical agent, drug, or biologic, (iii) a change in one or more of the amount, concentration, or dose titration of the pharmaceutical agent, drug, or biologic, and (iv) a change in the individual's emotional processing capabilities, a recommended treatment regimen, or recommending or determining a degree of effectiveness of at least one of a behavioral therapy, counseling, or physical exercise.

In any example herein, the App 214 can be configured to receive measurement data including physiological measurement data of an individual received from a physiological component, and/or data indicative of the response of an individual to a task and/or an interference rendered at a user interface of the apparatus 100 (as described in greater detail below), and/or data indicative of one or more of an amount, concentration, or dose titration, or other treatment regimen of a drug, pharmaceutical agent, biologic, or other medication being or to be administered to an individual.

Non-limiting examples of the computing device include a smartphone, a tablet, a slate, an e-reader, a digital assistant, or any other equivalent device, including any of the mobile communication devices described hereinabove. As an example, the computing device can include a processor unit that is configured to execute an application that includes an analysis module for analyzing the data indicative of the individual's response to the rendered tasks and/or interference (either or both with evocative element).

The example systems, methods, and apparatus can be implemented as a component in a product comprising a computing device that uses computer-implemented adaptive psychophysical procedures to assess human performance or delivers psychological/perceptual therapy.

A non-limiting example characteristic of a type of decision boundary metric that can be computed based on the response profile is the response criterion (a time-point measure), calculated using the standard procedure to calculate response criterion for a signal detection psychophysics assessment. See, e.g., Macmillan and Creelman (2004), “Signal Detection: A Users Guide” 2^{nd} edition, Lawrence Erlbaum USA.

In other non-limiting examples, the decision boundary metric may be more than a single quantitative measure but rather a curve defined by quantitative parameters based on which decision boundary metrics can be computed, such as but not limited to an area to one side or the other of the response profile curve. Other non-limiting example types of decision boundary metrics that can be computed to characterize the decision boundary curves for evaluating the time-varying characteristics of the decision process include a distance between the initial bias point (the starting point of the belief accumulation trajectory) and the criterion, a distance to the decision boundary, a “waiting cost” (e.g., the distance from the initial decision boundary and the maximum decision boundary, or the total area of the curve to that point), or the area between the decision boundary and the criterion line (including the area normalized to the response deadline to yield a measure of an “average decision boundary” or an “average criterion”). While examples herein may be described based on computation of a response criterion, other types of decision boundary metrics are applicable.

Following is a description of a non-limiting example use of a computational model of human decision-making (based on a drift diffusion model). While the drift diffusion model is used as the example, other types of models apply, including a Bayesian model. The drift-diffusion model (DDM) can be applied for systems with two-choice decision making. See, e.g., Ratcliff, R. (1978), “A theory of memory retrieval.” Psychological Review, 85, 59-108; Ratcliff, R., & Tuerlinckx, F. (2002), “Estimating parameters of the diffusion model: Approaches to dealing with contaminant reaction times and parameter variability,” Psychonomic Bulletin & Review, 9, 438-481. The diffusion model is based on an assumption that binary decision processes are driven by systematic and random influences.

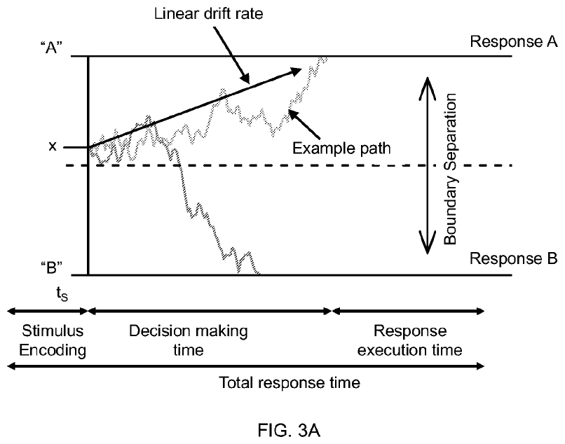

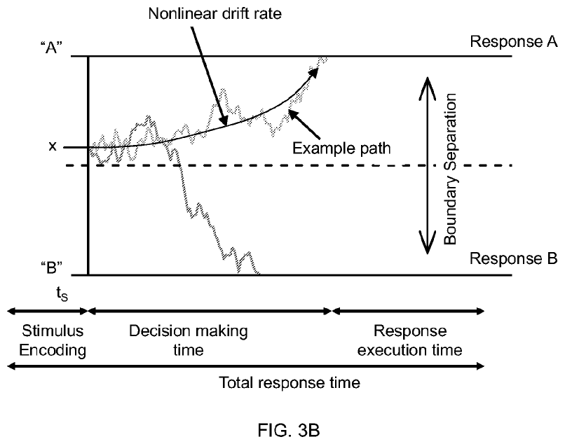

FIG. 3A shows an example plot of the diffusion model with a stimulus that results in a linear drift rate, showing example paths of the accumulation of belief from a stimulus. It shows the distributions of drift rates across trials for targets (signal) and non-targets (noise). The vertical line is the response criterion. The drift rate on each trial is determined by the distance between the drift criterion and a sample from the drift distribution. The process starts at point x, and moves over time until it reaches the lower threshold at “A” or the upper threshold at “B”. The DDM assumes that an individual is accumulating evidence for one or other of the alternative thresholds at each time step, and integrating that evidence to develop a belief, until a decision threshold is reached. Depending on which threshold is reached, different responses (i.e., Response A or Response B) are initiated by the individual. In a psychological application, this means that the decision process is finished and the response system is being activated, in which the individual initiates the corresponding response. As described in non-limiting examples below, this can require a physical action of the individual to actuate a component of the system or apparatus to provide the response (such as but not limited to tapping on the user interface in response to a target). The systematic influences are called the drift rate, and they drive the process in a given direction. The random influences add an erratic fluctuation to the constant path. With a given set of parameters, the model predicts distributions of process durations (i.e., response times) for the two possible outcomes of the process.

FIG. 3A also shows an example drift-diffusion path of the process, illustrating that the path is not straight but rather oscillates between the two boundaries, due to random influences. In a situation in which individuals are required to categorize stimuli, the process describes the ratio of information gathered over time that causes an individual to foster each of the two possible stimulus interpretations. Once belief points with sufficient clarity is reached, the individual initiates a response. In the example of FIG. 3A, processes reaching the upper threshold are indicative of a positive drift rate. In some trials, the random influences can outweigh the drift, and the process terminates at the lower threshold.

Example parameters of the drift diffusion model include quantifiers of the thresholds (“A” or “B”), the starting point (x), the drift rate, and a response time constant (t0). The DDM can provide a measure of conservatism, an indication that the process takes more time to reach one threshold and that it will reach the other threshold (opposite to the drift) less frequently. The starting point (x) provides an indicator of bias (reflecting differences in the amount of information that is required before the alternative responses are initiated). If x is closer to “A”, an individual requires a smaller (relative) amount of information to develop a belief to execute Response A, as compared with a larger (relative) amount of information that the individual would need to execute Response B. The smaller the distance between the starting point (x) and a threshold, the shorter the process durations would be for the individual to execute the corresponding response. A positive value of drift rate (v) serves as a measure of the mean rate of approach to the upper threshold (“A”). The drift rate indicates the relative amount of information per time unit that the individual absorbs information on a stimulus to develop a belief in order to initiate and execute a response. In an example, comparison of the drift rates computed from data of one individual to data from another can provide a measure of relative perceptual sensitivity of the individuals. In another example, comparison of the drift rates can provide a relative measure of task difficulty. For computation of the response time, the DDM allows for estimating their total duration, and the response time constant (t0) indicates the duration of extra-decisional processes. The DDM has been shown to describe accuracy and reaction times in human data for tasks. In the non-limiting example of FIG. 3A, the total response time is computed as a sum of the magnitude of time for stimulus encoding (tS), the time the individual takes for the decision, and the time for response execution.

As compared to the traditional drift diffusion model that is based on stimuli that result in linear drift rates, the example systems, methods, and apparatus according to the principles herein are configured to render stimuli that result in non-linear drift rates, which stimuli are based on tasks and/or interference (either or both with evocative element) that are time-varying and have specified response deadlines. As a result, the example systems, methods, and apparatus according to the principles herein are configured to apply a modified diffusion model (modified DDM) based on these stimuli that result in non-linear drift rates.

FIG. 3B shows an example plot of a non-linear drift rate in a drift diffusion computation. Example parameters of the modified DDM also include quantifiers of the thresholds (“A” or “B”), the starting point (x), the drift rate, and a response time constant (t0). Based on data collected from user interaction with the example systems, methods, and apparatus herein, the systems, methods, and apparatus are configured to apply the modified DDM with the non-linear drift rates to provide a measure of the conservatism or impulsivity of the strategy employed in the user interaction with the example platforms herein. The example systems, methods, and apparatus are configured to compute a measure of the conservatism or impulsivity of the strategy used by an individual based on the modified DDM model, to provide an indication of the time the process takes for a given individual to reach one threshold and as compared to reaching the other threshold (opposite to the drift). The starting point (x) in FIG. 3B also provides an indicator of bias (reflecting differences in the amount of information that is required before the alternative responses are initiated). For computation of the response time, the DDM allows for estimating their total duration, and the response time constant (t0) indicates the duration of extra-decisional processes.

In the example systems, methods, and apparatus according to the principles herein, the non-linear drift rate results from the time-varying nature of the stimuli, including (i) the time-varying feature of portions of the task and/or interference (either or both with evocative element) rendered to the user interface for user response (as a result of which the amount of information available for an individual to develop a belief is presented in a temporally non-linear manner), and (ii) the time limit of the response deadlines of the task and/or interference (either or both with evocative element), which can influence an individual's sense of timing to develop a belief in order to initiate a response. In this example as well, a positive value of drift rate (v) serves as a measure of the mean rate of approach to the upper threshold (“A”). The non-linear drift rate indicates the relative amount of information per time unit that the individual absorbs to develop a belief in order to initiate and execute a response. In an example, comparison of the drift rate computed from response data collected from one individual to the drift rate computed from response data collected from another individual can be used to provide a measure of relative perceptual sensitivity of the individuals. In another example, comparison of the drift rate computed from response data collected from a given individual from two or more different interaction sessions can be used to provide a relative measure of task difficulty. For computation of the response time of the individual's responses, the modified DDM also allows for estimating the total duration of the response time, and the response time constant (t0) indicates the duration of extra-decisional processes. In the non-limiting example of FIG. 3A, the total response time is computed as a sum of the magnitude of time for stimulus encoding (tS), the time the individual takes for the decision, and the time for response execution.

For the modified DDM, the distance between the thresholds (i.e., between “A” and “B”) provides a measure of conservatism—that is, the larger the separation, the more information is collected prior to an individual executing a response. The starting point (x) also provides an estimate of relative conservatism: if the process starts above or below the midpoint between the two thresholds, different amounts of information are required for both responses; that is, a more conservative decision criterion is applied for one response, and a more liberal criterion (i.e., impulsive) for the opposite response. The drift rate (v) indicates the (relative) amount of information gathered per time, denoting either perceptual sensitivity or task difficulty.

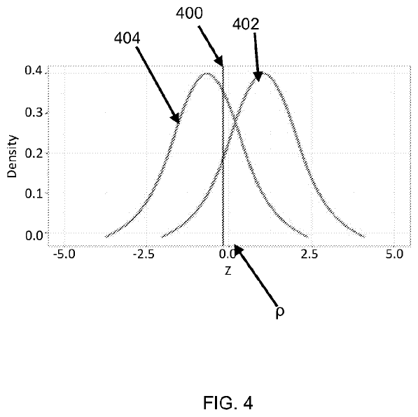

FIG. 4 shows an example plot of the signal (right curve 402) and noise (left curve 404) distributions of an individual or group psychophysical data, and the computed response criterion 400, based on data collected from an individual's responses with the tasks and/or interference rendered at a user interface of a computing device according to the principles herein (as described in greater detail hereinbelow). The intercept of the criterion line on the X axis (in Z units) can be used to provide an indication of the tendency of an individual to respond ‘yes’ (further right) or ‘no’ (further left). The response criterion 400 is left of the zero-bias decision point (p) and where the signal and noise distributions intersect. In the non-limiting example of FIG. 4, p is the location of the zero-bias decision on the decision axis in Z-units, and response criterion values to the left of p indicate an impulsive strategy and response criterion values to the right of p indicate a conservative strategy, with intercepts on the zero-bias point indicating a balanced strategy.

The example systems, methods, and apparatus according to the principles herein can be configured to compute a response criterion based on the detection or classification task(s) described herein that are composed of signal and non-signal response targets (as stimuli), in which a user indicates a response that indicates a feature, or multiple features, are present in a series of sequential presentations of stimuli or simultaneous presentation of stimuli.

The data indicative of the results of the classification of an individual according to the principles herein (including a classifier output) can be transmitted (with the pertinent consent) as a signal to one or more of a medical device, healthcare computing system, or other device, and/or to a medical practitioner, a health practitioner, a physical therapist, a behavioral therapist, a sports medicine practitioner, a pharmacist, or other practitioner, to allow formulation of a course of treatment for the individual or to modify an existing course of treatment, including to determine a change in one or more of an amount, concentration, or dose titration of a drug, biologic or other pharmaceutical agent being or to be administered to the individual and/or to determine an optimal type or combination of drug, biologic or other pharmaceutical agent to be administered to the individual.

The example systems, methods, and apparatus herein provide computerized classifiers, treatment tools, and other tools that can be used by a medical, behavioral, healthcare, or other professional as an aid in an assessment and/or enhancement of an individual's attention, working memory, and goal management. In an example implementation, the example systems, methods, and apparatus herein apply the modified DDM to the collected data to provide measures of conservatism or impulsivity. The example analysis performed using the example systems, methods, and apparatus according to the principles herein can be used to provide measures of attention deficits and impulsivity (including ADHD). The example systems, methods, and apparatus herein provide computerized classifiers, treatment tools, and other tools that can be used as aids in assessment and/or enhancement in other cognitive domains, such as but not limited to attention, memory, motor, reaction, executive function, decision-making, problem-solving, language processing, and comprehension. In some examples, the systems, methods, and apparatus can be used to compute measures for use for cognitive monitoring and/or disease monitoring. In some examples, the systems, methods, and apparatus can be used to compute measures for use for cognitive monitoring and/or disease monitoring during treatment of one or more cognitive conditions and/or diseases and/or executive function disorders.

An example system, method, and apparatus according to the principles herein can be configured to execute an example classifier to generate a quantifier of the cognitive skills in an individual. The example classifier can be built using a machine learning tool, such as but not limited to linear/logistic regression, principal component analysis, generalized linear mixed models, random decision forests, support vector machines, and/or artificial neural networks. In a non-limiting example, classification techniques that may be used to train a classifier using the performance measures of a labeled population of individuals (e.g, individuals with known cognitive disorders, executive function disorder, disease or other cognitive condition). The trained classifier can be applied to the computed values of the performance metric, to generate a classifier output indicative of a measure of cognition, a mood, a level of cognitive bias, or an affective bias of the individual. The trained classifier can be applied to measures of the responses of the individual to the tasks and/or interference (either or both with evocative element) to classify the individual as to a population label (e.g., cognitive disorder, executive function disorder, disease or other cognitive condition). In an example, machine learning may be implemented using cluster analysis. Each measurement of the cognitive response capabilities of participating individuals can be used as the parameter that groups the individuals to subsets or clusters. For example, the subset or cluster labels may be a diagnosis of a cognitive disorder, cognitive disorder, executive function disorder, disease or other cognitive condition. Using a cluster analysis, similarity metric of each subset and the separation between different subsets can be computed, and these similarity metrics may be applied to data indicative of an individual's responses to a task and/or interference (either or both with evocative element) to classify that individual to a subset. In another example, the classifier may be a supervised machine learning tool based on artificial neural networks. In such a case, the performance measures of individuals with known cognitive abilities may be used to train the neural network algorithm to model the complex relationships among the different performance measures. A trained classifier can be applied to the performance/response measures of a given individual to generate a classifier output indicative of the cognitive response capabilities of the individual. Other applicable techniques for generating a classifier include a regression or Monte Carlo technique for projecting cognitive abilities based on his/her cognitive performance. The classifier may be built using other data, including a physiological measure (e.g., EEG) and demographic measures.

In a non-limiting example, classification techniques that may be used to train a classifier using the performance measures of a labeled population of individuals, based on each individual's computed performance metrics, and other known outcome data on the individual, such as but not limited to outcome in the following categories: (i) an adverse event each individual experience in response to administration of a particular pharmaceutical agent, drug, or biologic; (ii) the amount, concentration, or dose titration of a pharmaceutical agent, drug, or biologic, administered to the individuals that resulted in a measurable or characterizable outcome for the individual (whether positive or negative); (iii) any change in the individual's emotional processing capabilities based on one or more interactions with the single-tasking and multi-tasking tasks rendered using the computing devices herein; (iv) a recommended treatment regimen, or recommending or determining a degree of effectiveness of at least one of a behavioral therapy, counseling, or physical exercise that resulted in a measurable or characterizable outcome for the individual (whether positive or negative); (v) the performance score of the individual at one or more of a cognitive test or a behavioral test, and (vi) the status or degree of progression of a cognitive condition, a disease or an executive function disorder of the individual. The example classifier can be trained based on the computed values of performance metrics of the known individuals, to be able to classify other yet-to-be classified individuals as to potential outcome in any of the possible categories.

In an example implementation, a programmed processing unit is configured to execute processor-executable instructions to render a task with an interference at a user interface. As described in greater detail herein, one or more of the task and the interference can be time-varying and have a response deadline, such that the user interface imposes a limited time period for receiving at least one type of response from the individual interacting with the apparatus or system. The processing unit is configured to control the user interface to measure data indicative of two or more differing types of responses to the task or to the interference. The programmed processing unit is further configured to execute processor-executable instructions to cause the example system or apparatus to receive data indicative of a first response of the individual to the task and a second response of the individual to the interference, analyze at least some portion of the data to compute at least one response profile representative of the performance of the individual, and determine a decision boundary metric (such as but not limited to the response criterion) from the response profile. The decision boundary metric (such as but not limited to the response criterion) can give a quantitative measure of a tendency of the individual to provide at least one type of response of the two or more differing types of responses (Response A vs. Response B) to the task or the interference. The programmed processing unit is further configured to execute processor-executable instructions to execute a classifier based on the computed values of the decision boundary metric (such as but not limited to the response criterion), to generate a classifier output indicative of the cognitive response capabilities of the individual.

In an example, the processing unit further uses the classifier output for one or more of changing one or more of the amount, concentration, or dose titration of the pharmaceutical agent, drug, biologic or other medication, identifying a likelihood of the individual experiencing an adverse event in response to administration of the pharmaceutical agent, drug, biologic or other medication, identifying a change in the individual's cognitive response capabilities, recommending a treatment regimen, or recommending or determining a degree of effectiveness of at least one of a behavioral therapy, counseling, or physical exercise.

In any example herein, the example classifier can be used as an intelligent proxy for quantifiable assessments of an individual's cognitive abilities. That is, once a classifier is trained, the classifier output can be used to provide the indication of the cognitive response capabilities of multiple individuals without use of other cognitive or behavioral assessment tests.

Monitoring cognitive deficits allows individuals, and/or medical, healthcare, behavioral, or other professional (with consent) to monitor the status or progression of a cognitive condition, a disease, or an executive function disorder. For example, individuals with Alzheimer's disease may shows mild symptoms initially, but others have more debilitating symptoms. If the status or progression of the cognitive symptoms can be regularly or periodically quantified, it can provide an indication of when a form of pharmaceutical agent or other drug may be administered or to indicate when quality of life might be compromised (such as the need for assisted living). Monitoring cognitive deficits also allows individuals, and/or medical, healthcare, behavioral, or other professional (with consent) to monitor the response of the individual to any treatment or intervention, particularly in cases where the intervention is known to be selectively effective for certain individuals. In an example, a cognitive assessment tool based on the classifiers herein can be an individual patient with attention deficit hyperactivity disorder (ADHD). In another example, the classifiers and other tools herein can be used as a monitor of the presence and/or severity of any cognitive side effects from therapies with known cognitive impact, such as but not limited to chemotherapy, or that involve uncharacterized or poorly characterized pharmacodynamics. In any example herein, the cognitive performance measurements and/or classifier analysis of the data may be performed every 30 minutes, each few hours, daily, two or more times per week, weekly, bi-weekly, each month, or once per year.

In an example, classifier can be used as an intelligent proxy for quantifiable measures of the performance of the individual under emotional load.

In a non-limiting example, the task and the interference can be rendered at the user interface such that the individual is required to provide the first response and the second response within a limited period of time. In an example, the individual is required to provide the first response and the second response substantially simultaneously.

In an example, the processing unit executes further instructions including applying at least one adaptive procedure to modify the task and/or the interference, such that analysis of the data indicative of the first response and/or the second response indicates a modification of the first response profile.

In an example, the processing unit controls the user interface to modify a temporal length of the response window associated with the response-deadline procedure.

In an example, the processing unit controls the user interface to modify a time-varying characteristics of an aspect of the task or the interference rendered to the user interface.

As described in connection with FIGS. 3A and 3B, the time-varying characteristics of the task and/or interference results in the time-varying availability of information about the target, such that that a linear drift-rate is no longer sufficient to capture development of belief over time (rather, requiring a nonlinear drift rate). A time-varying characteristic can be a feature such as, but not limited to, color, shape, type of creature, facial expression, or other feature that an individual requires in order to discriminate between a target and a non-target, resulting in differing time-characteristics of availability. The trial-by-trial adjustment of the response window length also can be a time-varying characteristic that alters the individual's perception of where the decision criteria needs to be in order to respond successfully to a task and/or an interference. Another time-varying characteristic that can be modified is the degree that an interference interferes with a parallel task which can introduce interruptions in belief accumulation and/or response selection and execution.

In an example, modifying the time-varying characteristics of an aspect of the task or the interference includes adjusting a temporal length of the rendering of the task or interference at the user interface between two or more sessions of interactions of the individual.

In an example, the time-varying characteristics is one or more of a speed of an object, a rate of change of a facial expression, a direction of trajectory of an object, a change of orientation of an object, at least one color of an object, a type of an object, or a size of an object.

In an example, the change in type of object is effected using morphing from a first type of object to a second type of object or rendering a blendshape as a proportionate combination of the first type of object and the second type of object.

In a non-limiting example, the processing unit can be configured to render a user interface or cause another component to execute least one element for indicating a reward to the individual for a degree of success in interacting with a task and/or interference, or another feature or other element of a system or apparatus. A reward computer element can be a computer generated feature that is delivered to a user to promote user satisfaction with the example system, method or apparatus, and as a result, increase positive user interaction and hence enjoyment of the experience of the individual.

In an example, the processing unit further computes as the classifier output parameters indicative of one or more of a bias sensitivity derived from the data indicative of the first response and the second response, a non-decision time sensitivity to parallel tasks, a belief accumulation sensitivity to parallel task demands, a reward rate sensitivity, or a response window estimation efficiency. Bias sensitivity can be a measure of how sensitive an individual is to certain of the tasks based on their bias (tendency to one type of response versus another (e.g., Response A vs. Response B)). Non-decision time sensitivity to parallel tasks can be a measure of how much the interference interferes with the individual's performance of the primary task. Belief accumulation sensitivity to parallel task demands can be a measure of the rate of the individual to develop/accumulate belief for responding to the interference during the individual's performance of the primary task. Reward rate sensitivity can be used to measure how an individual's response changes based on the temporal length of the response deadline window. When near the end of a response deadline window (e.g., as individual sees interference about to move off the field of view), the individual realizes that he is running out of time to make a decision. This measures how the individual's responses change accordingly. Response window estimation efficiency is explained as follows. When the individual is making a decision to act/respond or not act/no response, the decision needs to be based on when the individual thinks his time to respond is running out. For a varying window, the individual will not be able to measure that window perfectly, but with enough trials/session, based the response data, it may be possible to infer how good the individual is at making that estimation based on the time-varying aspect (e.g., trajectory) of the objects in the task or interference.

An example system, method, and apparatus according to the principles herein can be configured to train a predictive model of a measure of the cognitive capabilities of individuals based on feedback data from the output of the computational model of human decision-making for individuals that are previously classified as to the measure of cognitive abilities of interest. As used herein, the term “predictive model” encompasses models trained and developed based on models providing continuous output values and/or models based on discrete labels. In any example herein, the predictive model encompasses a classifier model. For example, the classifier can be trained using a plurality of training datasets, where each training dataset is associated with a previously classified individual from a group of individuals. Each of the training dataset includes data indicative of the first response of the classified individual to the task and data indicative of the second response of the classified individual to the interference, based on the classified individual's interaction with an example apparatus, system, or computing device described herein. The example classifier also can take as input data indicative of the performance of the classified individual at a cognitive test, and/or a behavioral test, and/or data indicative of a diagnosis of a status or progression of a cognitive condition, a disease, or a disorder (including an executive function disorder) of the classified individual.

In any example herein, the at least one processing unit can be programmed to cause an actuating component of the apparatus (including the cognitive platform) to effect auditory, tactile, or vibrational computerized elements to effect the stimulus or other interaction with the individual. In a non-limiting example, the at least one processing unit can be programmed to cause a component of the cognitive platform to receive data indicative of at least one response from the individual based on the user interaction with the task and/or interference, including responses provided using an input device. In an example where at least one graphical user interface is rendered to present the computerized stimulus to the individual, the at least one processing unit can be programmed to cause the graphical user interface to receive the data indicative of at least one response from the individual.

In any example herein, the data indicative of the response of the individual to a task and/or an interference can be measured using at least one sensor device contained in and/or coupled to an example system or apparatus herein, such as but not limited to a gyroscope, an accelerometer, a motion sensor, a position sensor, a pressure sensor, an optical sensor, an auditory sensor, a vibrational sensor, a video camera, a pressure-sensitive surface, a touch-sensitive surface, or other type of sensor. In other examples, the data indicative of the response of the individual to the task and/or an interference can be measured using other types of sensor devices, including a video camera, a microphone, joystick, keyboard, a mouse, a treadmill, elliptical, bicycle, steppers, or a gaming system (including a Wii®, a Playstation®, or an Xbox® or other gaming system). The data can be generated based on physical actions of the individual that are detected and/or measured using the at least one sensor device, as the individual executed a response to the stimuli presented with the task and/or interference.

The user may respond to tasks by interacting with the computer device. In an example, the user may execute a response using a keyboard for alpha-numeric or directional inputs; a mouse for GO/NO-GO clicking, screen location inputs, and movement inputs; a joystick for movement inputs, screen location inputs, and clicking inputs; a microphone for audio inputs; a camera for still or motion optical inputs; sensors such as accelerometer and gyroscopes for device movement inputs; among others. Non-limiting example inputs for a game system include but are not limited to a game controller for navigation and clicking inputs, a game controller with accelerometer and gryroscope inputs, and a camera for motion optical inputs. Example inputs for a mobile device or tablet include a touch screen for screen location information inputs, virtual keyboard alpha-numeric inputs, go/no go tapping inputs, and touch screen movement inputs; accelerometer and gyroscope motion inputs; a microphone for audio inputs; and a camera for still or motion optical inputs, among others. In other examples, data indicative of the individual's response can include physiological sensors/measures to incorporate inputs from the user's physical state, such as but not limited to electroencephalogram (EEG), magnetoencephalography (MEG), heart rate, heart rate variability, blood pressure, weight, eye movements, pupil dilation, electrodermal responses such as the galvanic skin response, blood glucose level, respiratory rate, and blood oxygenation.

In any example herein, the individual may be instructed to provide a response via a physical action of clicking a button and/or moving a cursor to a correct location on a screen, head movement, finger or hand movement, vocal response, eye movement, or other action of the individual.

As a non-limiting example, an individual's response to a task or interference rendered at the user interface that requires a user to navigate a course or environment or perform other visuo-motor activity may require the individual to make movements (such as but not limited to steering) that are detected and/or measured using at least one type of the sensor device. The data from the detection or measurement provides the response to the data indicative of the response.

As a non-limiting example, an individual's response to a task or interference rendered at the user interface that requires a user to discriminate between a target and a non-target may require the individual to make movements (such as but not limited to tapping or other spatially or temporally discriminating indication) that are detected and/or measured using at least one type of the sensor device. The data that is collected by a component of the system or apparatus based on the detection or other measurement of the individual's movements (such as but not limited to at least one sensor or other device or component described herein) provides the data indicative of the individual's responses.

The example system, method, and apparatus can be configured to apply the predictive model, using computational techniques and machine learning tools, such as but not limited to linear/logistic regression, principal component analysis, generalized linear mixed models, random decision forests, support vector machines, or artificial neural networks, to the data indicative of the individual's response to the tasks and/or interference, and/or data from one or more physiological measures, to create composite variables or profiles that are more sensitive than each measurement alone for generating a classifier output indicative of the cognitive response capabilities of the individual. In an example, the classifier output can be configured for other indications such as but not limited to detecting an indication of a disease, disorder or cognitive condition, or assessing cognitive health

The example classifiers herein can be trained to be applied to data collected from interaction sessions of individuals with the cognitive platform to provide the output. In a non-limiting example, the predictive model can be used to generate a standards table, which can be applied to the data collected from the individual's response to task and/or interference to classify the individual's cognitive response capabilities.

Non-limiting examples of assessment of cognitive abilities include assessment scales or surveys such as the Mini Mental State Exam, CANTAB cognitive battery, Test of Variables of Attention (TOVA), Repeatable Battery for the Assessment of Neuropsychological Status, Clinical Global Impression scales relevant to specific conditions, Clinician's Interview-Based Impression of Change, Severe Impairment Battery, Alzheimer's Disease Assessment Scale, Positive and Negative Syndrome Scale, Schizophrenia Cognition Rating Scale, Conners Adult ADHD Rating Scales, Hamilton Rating Scale for Depression, Hamilton Anxiety Scale, Montgomery-Asberg Depressing Rating scale, Young Mania Rating Scale, Children's Depression Rating Scale, Penn State Worry Questionnaire, Hospital Anxiety and Depression Scale, Aberrant Behavior Checklist, Activities for Daily Living scales, ADHD self-report scale, Positive and Negative Affect Schedule, Depression Anxiety Stress Scales, Quick Inventory of Depressive Symptomatology, and PTSD Checklist.

In other examples, the assessment may test specific functions of a range of cognitions in cognitive or behavioral studies, including tests for perceptive abilities, reaction and other motor functions, visual acuity, long-term memory, working memory, short-term memory, logic, and decision-making, and other specific example measurements, including but are not limited to TOVA, MOT (motion-object tracking), SART, CDT (Change detection task), UFOV (useful Field of view), Filter task, WAIS digit symbol, Troop, Simon task, Attentional Blink, N-back task, PRP task, task-switching test, and Flanker task.

In non-limiting examples, the example systems, methods, and apparatus according to the principles described herein can be applicable to many different types of neuropsychological conditions, such as but not limited to dementia, Parkinson's disease, cerebral amyloid angiopathy, familial amyloid neuropathy, Huntington's disease, or other neurodegenerative condition, autism spectrum disorder (ASD), presence of the 16p11.2 duplication, and/or an executive function disorder, such as but not limited to attention deficit hyperactivity disorder (ADHD), sensory-processing disorder (SPD), mild cognitive impairment (MCI), Alzheimer's disease, multiple sclerosis, schizophrenia, major depressive disorder (MDD), or anxiety (including social anxiety), bipolar disorder, post-traumatic stress disorder, schizophrenia, dementia, Alzheimer's disease, or multiple sclerosis.

The instant disclosure is directed to computer-implemented devices formed as example cognitive platforms configured to implement software and/or other processor-executable instructions for the purpose of measuring data indicative of a user's performance at one or more tasks, to provide a user performance metric. The example performance metric can be used to derive an assessment of a user's cognitive abilities under emotional load and/or to measure a user's response to a cognitive treatment, and/or to provide data or other quantitative indicia of a user's condition (including physiological condition and/or cognitive condition). Non-limiting example cognitive platforms according to the principles herein can be configured to classify an individual as to a neuropsychological condition, autism spectrum disorder (ASD), presence of the 16p11.2 duplication, and/or an executive function disorder, and/or potential efficacy of use of the cognitive platform when the individual is being administered (or about to be administered) a drug, biologic or other pharmaceutical agent, based on the data collected from the individual's interaction with the cognitive platform and/or metrics computed based on the analysis (and associated computations) of that data. Yet other non-limiting example cognitive platforms according to the principles herein can be configured to classify an individual as to likelihood of onset and/or stage of progression of a neuropsychological condition, including as to a neurodegenerative condition, based on the data collected from the individual's interaction with the cognitive platform and/or metrics computed based on the analysis (and associated computations) of that data. The neurodegenerative condition can be, but is not limited to, Alzheimer's disease, dementia, Parkinson's disease, cerebral amyloid angiopathy, familial amyloid neuropathy, or Huntington's disease.

Any classification of an individual as to likelihood of onset and/or stage of progression of a neurodegenerative condition according to the principles herein can be transmitted as a signal to a medical device, healthcare computing system, or other device, and/or to a medical practitioner, a health practitioner, a physical therapist, a behavioral therapist, a sports medicine practitioner, a pharmacist, or other practitioner, to allow formulation of a course of treatment for the individual or to modify an existing course of treatment, including to determine a change in dosage of a drug, biologic or other pharmaceutical agent to the individual or to determine an optimal type or combination of drug, biologic or other pharmaceutical agent to the individual.

In any example herein, the cognitive platform can be configured as any combination of a medical device platform, a monitoring device platform, a screening device platform, or other device platform.

The instant disclosure is also directed to example systems that include cognitive platforms that are configured for coupling with one or more physiological or monitoring component and/or cognitive testing component. In some examples, the systems include cognitive platforms that are integrated with the one or more other physiological or monitoring component and/or cognitive testing component. In other examples, the systems include cognitive platforms that are separately housed from and configured for communicating with the one or more physiological or monitoring component and/or cognitive testing component, to receive data indicative of measurements made using such one or more components.

In an example system, method, and apparatus herein, the processing unit can be programmed to control the user interface to modify a temporal length of the response window associated with a response-deadline procedure.

In an example system, method, and apparatus herein, the processing unit can be configured to control the user interface to modify a time-varying characteristics of an aspect of the task or the interference rendered to the user interface. For example, modifying the time-varying characteristics of an aspect of the task or the interference can include adjusting a temporal length of the rendering of the task or interference at the user interface between two or more sessions of interactions of the individual. As another example, the time-varying characteristics is one or more of a speed of an object, a rate of change of a facial expression, a direction of trajectory of an object, a change of orientation of an object, at least one color of an object, a type of an object, or a size of an object. In any example herein, the foregoing time-varying characteristic can be applied to an object that includes the evocative element to modify an emotional load of the individual's interaction with the apparatus (e.g., computing device or cognitive platform).

In an example system, method, and apparatus herein, the change in type of object is effected using morphing from a first type of object to a second type of object or rendering a blendshape as a proportionate combination of the first type of object and the second type of object.

In an example system, method, and apparatus herein, the processing unit can be further programmed to compute as the classifier output parameters indicative of one or more of a bias sensitivity derived from the data indicative of the first response and the second response, a non-decision time sensitivity to parallel tasks, a belief accumulation sensitivity to parallel task demands, a reward rate sensitivity, or a response window estimation efficiency.

In an example system, method, and apparatus herein, the processing unit can be further programmed to control the user interface to render the task as a continuous visuo-motor tracking task.

In an example system, method, and apparatus herein, the processing unit controls the user interface to render the interference as a target discrimination task.

As used herein, a target discrimination task may also be referred to as a perceptual reaction task, in which the individual is instructed to perform a two-feature reaction task including target stimuli and non-target stimuli through a specified form of response. As a non-limiting example, that specified type of response can be for the individual to make a specified physical action in response to a target stimulus (e.g., move or change the orientation of a device, tap on a sensor-coupled surface such as a screen, move relative to an optical sensor, make a sound, or other physical action that activates a sensor device) and refrain from making such specified physical action in response to a non-target stimulus.

In a non-limiting example, the individual is required to perform a visuo-motor task (as a primary task) with a target discrimination task as an interference (secondary task) (either or both including an evocative element). To effect the visuo-motor task, a programmed processing unit renders visual stimuli that require fine motor movement as reaction of the individual to the stimuli. In some examples, the visuo-motor task is a continuous visuo-motor task. The processing unit is programmed to alter the visual stimuli and recording data indicative of the motor movements of the individual over time (e.g., at regular intervals including 1, 5, 10, or 30 times per second). Example stimuli rendered using the programmed processing unit for a visuo-motor task requiring fine motor movement may be a visual presentation of a path that an avatar is required to remain within. The programmed processing unit may render the path with certain types of obstacles that the individual is either required to avoid or to navigate towards. In an example, the fine motor movements effect by the individual, such as but not limited to tilting or rotating a device, are measured using an accelerometer and/or a gyroscope (e.g., to steer or otherwise guide the avatar on the path while avoiding or crossing the obstacles as specified). The target discrimination task (serving as the interference), can be based on targets and non-targets that differ in a non-evocative feature (such as but not limited to the shape and/or the color).

In any example, the apparatus may be configured to instruct the individual to provide the response to the evocative element as an action that is read by one or more sensors (such as a movement that is sensed using a gyroscope or accelerometer or a motion or position sensor, or a touch that is sensed using a touch-sensitive, pressure sensitive or capacitance-sensitive sensor.

In some examples, the task and/or interference can be a visuo-motor task, a target discrimination task, and/or a memory task.

Within the context of a computer-implemented adaptive response-deadline procedure, the response-deadline can be adjusted between trials or blocks of trials to manipulate the individual's performance characteristics towards certain goals. A common goal is driving the individual's average response accuracy towards a certain value by controlling the response deadline.

In a non-limiting example, the hit rate may be defined as the number of correct responses to a target stimuli divided by the total number of target stimuli presented, or the false alarm rate (e.g., the number of responses to a distractor stimuli divided by the number of distractor stimuli presented), the miss rate (e.g., the number of nonresponses to a target stimuli divided by the number of incorrect responses, including the nonresponses to a target stimuli added to the number of responses to a distractor stimuli), the correct response rate (the proportion of correct responses not containing signal). In an example, the correct response rate may be calculated as the number of non-responses to the distractor stimuli divided by the number of non-responses to the distractor stimuli plus the number of responses to the target stimuli.

An example system, method, and apparatus according to the principles herein can be configured to apply adaptive performance procedures to modify measures of performance to a specific stimulus intensity. The procedure can be adapted based on a percent correct (PC) signal detection metric of sensitivity to a target. In an example system, the value of percent correct (i.e., percent of correct responses of the individual to a task or evocative element) may be used in the adaptive algorithms as the basis for adapting the stimulus level of tasks and/or interferences rendered at the user interface for user interaction from one trial to another. An adaptive procedure based on a computational model of human decision-making (such as but not limited to the modified DDM), classifiers built from outputs of such models, and the analysis described herein based on the output of the computational model, can be more quantitatively informative on individual differences or on changes in sensitivity to a specific stimulus level. The performance metric provides a flexible tool for determining a performance of the individual under emotional load. Accordingly, an adaptation procedure based on performance metric measurements at the individual or group level become a desirable source of information about the changes in performance at the individual or group level over time with repeated interactions with the tasks and evocative elements described herein, and measurements of the individual's responses with the interactions.

Executive function training, such as that delivered by the example systems, methods, and apparatus described herein can be configured to apply an adaptive algorithm to modify the stimulus levels (including emotional load based on the evocative element(s) implemented) between trials, to move a user's performance metric to the desired level (value), depending on the needs or preference of the individual or based on the clinical population receiving the treatment.

The example systems, methods, and apparatus described herein can be configured to apply an adaptive algorithm that is adapted based on the computed performance metric as described herein to modify the difficulty levels of the tasks and/or interference (either or both including an evocative element) rendered at the user interface for user interaction from one trial to another.

In an example, the task and/or interference (either or both including an evocative element) can be modified/adjusted/adapted based on an iterative estimation of metrics by tracking current estimates and selecting the features, trajectory, and response window of the targeting task, and level/type of parallel task interference for the next trial in order to maximize information the trial can provide.

In some examples, the task and/or interference (either or both including an evocative element) are adaptive tasks. The task and/or interference can be adapted or modified in difficulty level based on the performance metric, as described hereinabove. Such difficulty adaptation may be used to determine the ability of the participant.

In an example, the difficulty of the task (potentially including an evocative element) adapts with every stimuli that is presented, which could occur more often than once at regular time intervals (e.g., every 5 seconds, every 10 seconds, every 20 seconds or other regular schedule).

In another example, the difficulty of a continuous task (potentially including an evocative element) can be adapted on a set schedule, such as but not limited to every 30 seconds, 10 seconds, 1 second, 2 times per second, or 30 times per second.

In an example, the length of time of a trial depends on the number of iterations of rendering (of the tasks/interference) and receiving (of the individual's responses) and can vary in time. In an example, a trial can be on the order of about 500 milliseconds, about 1 second (s), about 10 s, about 20 s, about 25 s, about 30 s, about 45 s, about 60 s, about 2 minutes, about 3 minutes, about 4 minutes, about 5 minutes, or greater. Each trial may have a pre-set length or may be dynamically set by the processing unit (e.g., dependent on an individual's performance level or a requirement of the adapting from one level to another).

In an example, the task and/or interference (either or both including an evocative element) can be modified based on targeting changes in one or more specific metrics by selecting features, trajectory, and response window of the targeting task, and level/type of parallel task interference to progressively require improvements in those metrics in order for the apparatus to indicate to an individual that they have successfully performed the task. This could include specific reinforcement, including explicit messaging, to guide the individual to modify performance according to the desired goals.

In an example, the task and/or interference (either or both including an evocative element) can be modified based on a comparison of an individual's performance with normative data or a computer model or taking user input (the individual perfoiming the task/interference or another individual such as a clinician) to select a set of metrics to target for changing in a specific order, and iteratively modifying this procedure based on the subject's response to treatment. This could include feedback to the individual performing the task/interference or another individual as notification of changes to the procedure, potentially enabling them to approve or modify these changes before they take effect.

In various examples, the difficulty level may be kept constant or may be varied over at least a portion of a session in an adaptive implementation, where the adaptive task (primary task or secondary task) increases or decreases in difficulty based on the performance metric.

An example system, method, and apparatus according to the principles herein can be configured to enhance the cognitive skills in an individual. In an example implementation, a programmed processing unit is configured to execute processor-executable instructions to render a task with an interference at a user interface. As described in greater detail herein, one or more of the task and the interference (either or both including an evocative element) can be time-varying and have a response deadline, such that the user interface imposes a limited time period for receiving at least one type of response from the individual interacting with the apparatus or system.

An example processing unit is configured to control the user interface to render a first instance of a task with an interference at the user interface, requiring a first response from the individual to the first instance of the task in the presence of the interference and a response from the individual to at least one evocative element. Either or both of the first instance of the task and the interference includes at least one an evocative element. The user interface can be configured to measure data indicative of the response of the individual to the at least one evocative element, the data including at least one measure of emotional processing capabilities of the individual under emotional load. The example processing unit is configured to measure substantially simultaneously the first response from the individual to the first instance of the task and the response from the individual to the at least one evocative element, and to receive data indicative of the first response and the response of the individual to the at least one evocative element. The example processing unit is also configured to analyze the data indicative of the first response and the response of the individual to the at least one evocative element to compute at least one performance metric comprising at least one quantified indicator of cognitive abilities of the individual under emotional load.

In an example, the indication of the modification of the cognitive response capabilities can be based on observation of a change in a measure of a degree of impulsiveness or conservativeness of the individual's cognitive response capabilities.

In an example, the indication of the modification of the cognitive abilities under emotional load can include a change in a measure of one or more of affective bias, mood, level of cognitive bias, sustained attention, selective attention, attention deficit, impulsivity, inhibition, perceptive abilities, reaction and other motor functions, visual acuity, long-term memory, working memory, short-term memory, logic, and decision-making.

In an example, adapting the task and/or interference based on the first performance metric includes one or more of modifying the temporal length of the response window, modifying a type of reward or rate of presentation of rewards to the individual, and modifying a time-varying characteristic of the task and/or interference (including the evocative element).

In an example, modifying the time-varying characteristics of an aspect of the task or the interference (including the evocative element) can include adjusting a temporal length of the rendering of the task or interference at the user interface between two or more sessions of interactions of the individual.

In an example, the time-varying characteristics can include one or more of a speed of an object, a rate of change of a facial expression, a direction of trajectory of an object, a change of orientation of an object, at least one color of an object, a type of an object, or a size of an object, or modifying a sequence or balance of rendering of targets versus non-targets at the user interface.

In an example, the change in type of object is effected using morphing from a first type of object to a second type of object or rendering a blendshape as a proportionate combination of the first type of object and the second type of object.

Designing the computer-implemented adaptive procedure using a goal of explicitly measuring the shape and/or area of the decision boundary, the response deadlines can be adjusted to points where measurements produce maximal information of use for defining this boundary. These optimal deadlines may be determined using an information theoretic approach to minimize the expected information entropy.

Example systems, methods and apparatus according to the principles herein can be implemented using a programmed computing device including at least one processing unit, to determine a potential biomarker for clinical populations.

Example systems, methods and apparatus according to the principles herein can be implemented using a programmed computing device including at least one processing unit to measure change in response profile in individuals or groups after use of an intervention.

Example systems, methods and apparatus according to the principles herein can be implemented using a programmed computing device including at least one processing unit to apply the example metrics herein, to add another measurable characteristic of individual or group data that can be implemented for greater measurement of psychophysical-threshold accuracy and assessment of response profile to computer-implemented adaptive psychophysical procedures.

Example systems, methods and apparatus according to the principles herein can be implemented using a programmed computing device including at least one processing unit to apply the example metrics herein to add a new dimension to available data that can be used to increase the amount of information harvested from psychophysical testing.

An example system, method, and apparatus according to the principles herein can be configured to enhance the cognitive skills in an individual. In an example implementation, a programmed processing unit is configured to execute processor-executable instructions to render a task with an interference at a user interface. As described in greater detail herein, one or more of the task and the interference can be time-varying and have a response deadline, such that the user interface imposes a limited time period for receiving at least one type of response from the individual interacting with the apparatus or system. An example processing unit is configured to control the user interface to render a first instance of a task with an interference at the user interface, requiring a first response from the individual to the first instance of the task in the presence of the interference and a response from the individual to at least one evocative element. Either or both of the first instance of the task and the interference includes at least one an evocative element. The user interface can be configured to measure data indicative of the response of the individual to the at least one evocative element, the data including at least one measure of emotional processing capabilities of the individual under emotional load. The example processing unit is configured to measure substantially simultaneously the first response from the individual to the first instance of the task and the response from the individual to the at least one evocative element, and to receive data indicative of the first response and the response of the individual to the at least one evocative element. The example processing unit is also configured to analyze the data indicative of the first response and the response of the individual to the at least one evocative element to compute a first performance metric comprising at least one quantified indicator of cognitive abilities of the individual under emotional load. The programmed processing unit is further configured to adjust a difficulty of one or more of the task and the interference based on the computed at least one first performance metric such that the apparatus renders the task with the interference at a second difficulty level, and compute a second performance metric representative of cognitive abilities of the individual under emotional load based at least in part on the data indicative of the first response and the response of the individual to the at least one evocative element.

Another example system, method, and apparatus according to the principles herein can be configured to enhance the cognitive skills in an individual. In an example implementation, a programmed processing unit is configured to execute processor-executable instructions to render a task with an interference at a user interface. As described in greater detail herein, one or more of the task and the interference can be time-varying and have a response deadline, such that the user interface imposes a limited time period for receiving at least one type of response from the individual interacting with the apparatus or system. An example processing unit is configured to control the user interface to render a first instance of a task with an interference at the user interface, requiring a first response from the individual to the first instance of the task in the presence of the interference and a response from the individual to at least one evocative element. Either or both of the first instance of the task and the interference includes at least one an evocative element. The user interface can be configured to measure data indicative of the response of the individual to the at least one evocative element, the data including at least one measure of emotional processing capabilities of the individual under emotional load. The example processing unit is configured to measure substantially simultaneously the first response from the individual to the first instance of the task and the response from the individual to the at least one evocative element, and to receive data indicative of the first response and the response of the individual to the at least one evocative element. The example processing unit is also configured to analyze the data indicative of the first response and the response of the individual to the at least one evocative element to compute at least one performance metric comprising at least one quantified indicator of cognitive abilities of the individual under emotional load. Based at least in part on the at least one performance metric, the example processing unit is also configured to generate an output to the user interface indicative of at least one of: (i) a likelihood of the individual experiencing an adverse event in response to administration of the pharmaceutical agent, drug, or biologic, (ii) a recommended change in one or more of the amount, concentration, or dose titration of the pharmaceutical agent, drug, or biologic, (iii) a change in the individual's cognitive response capabilities, (iv) a recommended treatment regimen, or (v) a recommended or determined degree of effectiveness of at least one of a behavioral therapy, counseling, or physical exercise.

In a non-limiting example, the processing unit can be further configured to measure substantially simultaneously the first response from the individual to the first instance of the task, a secondary response of the individual to the interference, and the response to the at least one evocative element.

In a non-limiting example, the processing unit can be further configured to output to the individual or transmits to a computing device the computed at least one performance metric.

In a non-limiting example, the processing unit can be further configured to render a second instance of the task at the user interface, requiring a second response from the individual to the second instance of the task, and analyze a difference between the data indicative of the first response and the second response to compute an interference cost as a measure of at least one additional indication of cognitive abilities of the individual.

In a non-limiting example, based on the results of the analysis of the performance metrics, a medical, healthcare, or other professional (with consent of the individual) can gain a better understanding of potential adverse events which may occur (or potentially are occurring) if the individual is administered a particular type of, amount, concentration, or dose titration of a pharmaceutical agent, drug, biologic, or other medication, including potentially affecting cognition.

In a non-limiting example, a searchable database is provided herein that includes data indicative of the results of the analysis of the performance metrics for particular individuals, along with known levels of efficacy of at least one types of pharmaceutical agent, drug, biologic, or other medication experiences by the individuals, and/or quantifiable information on one or more adverse events experienced by the individual with administration of the at least one types of pharmaceutical agent, drug, biologic, or other medication. The searchable database can be configured to provide metrics for use to determine whether a given individual is a candidate for benefiting from a particular type of pharmaceutical agent, drug, biologic, or other medication based on the performance metrics, response measures, response profiles, and/or decision boundary metric (such as but not limited to response criteria) obtained for the individual in interacting with the task and/or interference rendered at the computing device.

As a non-limiting example, performance metrics can assist with identifying whether the individual is a candidate for a particular type of drug (such as but not limited to a stimulant, e.g., methylphenidate or amphetamine) or whether it might be beneficial for the individual to have the drug administered in conjunction with a regiment of specified repeated interactions with the tasks and/or interference rendered to the computing device. Other non-limiting examples of a biologic, drug or other pharmaceutical agent applicable to any example described herein include methylphenidate (MPH), scopolamine, donepezil hydrochloride, rivastigmine tartrate, memantine HCl, solanezumab, aducanumab, and crenezumab.

In a non-limiting example, based on the results of the analysis of the performance metric, a medical, healthcare, or other professional (with consent of the individual) can gain a better understanding of potential adverse events which may occur (or potentially are occurring) if the individual is administered a different amount, concentration, or dose titration of a pharmaceutical agent, drug, biologic, or other medication, including potentially affecting cognition.

In a non-limiting example, a searchable database is provided herein that includes data indicative of the results of the analysis of the performance metrics for particular individuals, along with known levels of efficacy of at least one types of pharmaceutical agent, drug, biologic, or other medication experiences by the individuals, and/or quantifiable information on one or more adverse events experienced by the individual with administration of the at least one types of pharmaceutical agent, drug, biologic, or other medication. The searchable database can be configured to provide metrics for use to determine whether a given individual is a candidate for benefiting from a particular type of pharmaceutical agent, drug, biologic, or other medication based on the response measures, response profiles, and/or decision boundary metric (such as but not limited to response criteria) obtained for the individual in interacting with the task and/or interference rendered at the computing device. As a non-limiting example, based on data indicative of a user interaction with the tasks and/or interference (including the evocative element) rendered at a user interface of a computing device, the performance metrics could provide information on the individual, based on the cognitive capabilities of the individual under emotional load. This data can assist with identifying whether the individual is a candidate for a particular type of drug (such as but not limited to a stimulant, e.g., methylphenidate or amphetamine) or whether it might be beneficial for the individual to have the drug administered in conjunction with a regiment of specified repeated interactions with the tasks and/or interference rendered to the computing device. Other non-limiting examples of a biologic, drug or other pharmaceutical agent applicable to any example described herein include methylphenidate (MPH), scopolamine, donepezil hydrochloride, rivastigmine tartrate, memantine HCl, solanezumab, aducanumab, and crenezumab.

In an example, the change in the individual's cognitive response capabilities comprises an indication of a change in degree of impulsiveness or conservativeness of the individual's cognitive response strategy.

As a non-limiting example, given that impulsive behavior is attendant with ADHD, an example cognitive platform that is configured for delivering treatment (including of executive function) may promote less impulsive behavior in a regimen. This may target dopamine systems in the brain, increasing normal regulation, which may result in a transfer of benefits of the reduction of impulsive behavior to the everyday life of an individual.

Stimulants such as methylphenidate and amphetamine are also administered to individuals with ADHD, to increase levels of norepinephrine and dopamine in the brain. Their cognitive effects may be attributed to their actions at the prefrontal cortex, however, there may not be remediation of cognitive control deficits or other cognitive abilities. An example cognitive platform herein can be configured for delivering treatment (including of executive function) to remediate an individual's cognitive control deficit.

The use of the example systems, methods, and apparatus according to the principles described herein can be applicable to many different types of neuropsychological conditions, such as but not limited to dementia, Parkinson's disease, cerebral amyloid angiopathy, familial amyloid neuropathy, Huntington's disease, or other neurodegenerative condition, autism spectrum disorder (ASD), presence of the 16p11.2 duplication, and/or an executive function disorder, such as but not limited to attention deficit hyperactivity disorder (ADHD), sensory-processing disorder (SPD), mild cognitive impairment (MCI), Alzheimer's disease, multiple sclerosis, schizophrenia, major depressive disorder (MDD), or anxiety.

In any example implementation, data and other information from an individual is collected, transmitted, and analyzed with their consent.

As a non-limiting example, the cognitive platform described in connection with any example system, method and apparatus herein, including a cognitive platform based on interference processing, can be based on or include the Project: EVO™ platform by Akili Interactive Labs, Inc., Boston, Mass.

Non-limiting Example Tasks and Interference Under Emotional Load

Following is a summary of reported results showing the extensive physiological, behavioral, and cognitive measurements data and analysis of the regions of the brain, neural activity, and/or neural pathways mechanisms involved (e.g., activated or suppressed) as an individual interact with emotional or affective stimuli under differing emotional load. The articles also described the differences that can be sensed and quantifiably measured based on the individual's performance at cognitive tasks versus stimuli with evocative elements (e.g., emotional or affective elements).

Based on physiological and other measurements, regions of the brain implicated in emotional processing, cognitive tasks, and tasks under emotional load, are reported. For example, in the review article by Pourtois et al., 2013, “Brain mechanisms for emotional influences on perception and attention: What is magic and what is not,” Biological Psychology, 92, 492-512, it is reported that the amygdala monitors the emotional value of stimuli, projects to several other areas of the brain, and sends feedback to sensory pathways (including striate and extrastriate visual cortex). It is also reported that, due to an individual's limited processing capacity, the individual cannot fully analyze simultaneous stimuli in parallel, and these stimuli compete for processing resources in order to gain access to higher cognitive stages and awareness of the individual. With an individual having to direct attention to the location or features of a given stimulus, neural activity in brain regions representing this stimulus increases, at the expense of other concurrent stimuli. Pourtois et al. indicates that this phenomenon has been extensively demonstrated by neuronal recordings as well as imaging methods (EEG, PET, fMRI), and attributed to a gain control. Pourtois et al. concludes that emotion signals may enhance processing efficiency and competitive strength of emotionally significant events through gain control mechanisms similar to those of other attentional systems, but mediated by distinct neural mechanisms in the amygdala and interconnected prefrontal areas, and indicate that alterations in these brain mechanisms might be associated with psychopathological conditions, such as anxiety or phobia. It is also reported that anxious or depressed patients can show maladaptive attentional biases towards negative information. Pourtois et al. also reports that imaging results from EEG and fMRI support a conclusion that the processing of emotional (such as fearful or threat-related) stimuli yields a gain control effect in the visual cortex and the emotional gain control effect can account for the more efficient processing of threat-related stimuli, in addition to or in parallel with any concurrent modulation by other task-dependent or exogenous stimulus-driven mechanisms of attention (see also Brosch et al., 2011, “Additive effects of emotional, endogenous, and exogenous attention: behavioral and electrophysiological evidence,” Neuropsychologia 49, 1779-1787).

Results of studies in healthy adult participants using magnetoencephalography (MEG) and source localization techniques are also reported (Pourtois et al., 2010, “Emotional automaticity is a matter of timing,” J. Neurosci. 30 (17), 5825-5829) The source localization techniques applied with the MEG allows for accurate imaging of the activity of deep brain structures. In the study, the participants performed a line discrimination task (i.e. matching the orientation of two line flankers shown on each side of a central face), where the line discrimination task was either easy (low load) or difficult (high load), while the central face could have either a fearful or neutral expression. The MEG imaging results showed that the amygdala responded more to fearful relative to neutral faces early after stimulus onset (40-140 ms) regardless of task load, but this amygdala response was modulated by load during a later time interval only (280-410 ms). Pourtois et al. also reports behavioral results which confirmed that emotion (e.g. seeing a fearful face) can improve fast temporal vision (via magnocellular channels) at the expense of fine-grained spatial vision (dependent on parvocellular channels. It is also reported that visual detection and attention are boosted for emotional (e.g. threat) relative to neutral stimuli, where such effects are manifested by (and can be measured based on) faster reaction times (RTs) and/or enhanced accuracy in various tasks. The behavior is reported for visual search tasks (see, e.g., Dominguez-Borràs et al., 2013, “Affective biases in attention and perception,” Handbook of Human Affective Neuroscience, 331-356, Cambridge University Press, NY; Eastwood et al., 2003, “Negative facial expression captures attention and disrupts performance,” Percept. Psychophys. 65 (3), 352-358; Williams et al., 2005, “Look at me, I'm smiling: visual search for threatening and nonthreatening facial expressions,” Visual Cognition 12 (1), 29-50); attentional blink tasks (see Anderson, A. K., 2005, “Affective influences on the attentional dynamics supporting awareness,” Journal Experimental Psychology General, 134 (2), 258-281, and Anderson et al., 2001, “Lesions of the human amygdala impair enhanced perception of emotionally salient events,” Nature 411 (6835), 305-309.); and spatial orienting tasks (Brosch et al., 2011, “Additive effects of emotional, endogenous, and exogenous attention: behavioral and electrophysiological evidence,” Neuropsychologia 49, 1779-1787; Pourtois et al., 2004, “Electrophysiological correlates of rapid spatial orienting towards fearful faces,” Cerebral Cortex 14 (6), 619-633). Pourtois et al. also reports that the role for the amygdala and emotional influences on attention in these tasks is supported by the convergence of these behavioral effects in healthy participants with patterns of neurophysiological responses in imaging studies, as well as observations in patients with lesions to the amygdala. Pourtois et al. points out that the reported observation of changes in behavior (RT or accuracy) combined with the reported neuropsychology case studies and imaging work (EEG, MEG or fMRI) provide useful insight into activations in specific brain systems and help to identify mechanisms underlying emotional attention.

The physiological measurements reported in Pourtois et al. indicates that the requirement of the individual to perform a task under emotional load (by virtue of the presence of the faces with the fearful or neutral expression as the individual performs the task) can introduce a quantifiable difference in the individual's performance of the task, e.g., differences in reaction time and accuracy.

Based on physiological and other measurements, it is also reported that emotional load can affect an individual's performance at cognitive tasks versus tasks involving emotional or affective stimuli.

For example, Pourtois et al. reports that both emotional influences from the amygdala and attentional influences from fronto-parietal areas seem to act as distinct gain control systems that can amplify emotion or task-relevant information in a stimulus-specific manner, producing similar increases in fMRI and EEG responses (Lang et al., 1998, “Neural correlates of levels of emotional awareness: evidence of an interaction between emotion and attention in the anterior cingulate cortex,” Journal of Cognitive Neuroscience 10 (4), 525-535; Sabatinelli et al., 2009, “The timing of emotional discrimination in human amygdala and ventral visual cortex,” Journal of Neuroscience 29 (47), 14864-14868). It is reported that, because the emotion and attention effects have distinct sources, they can occur in a parallel or competitive manner and produce additive (or occasionally interactive) effects on an individual's sensory responses (see, e.g., Vuilleumier et al., 2001, “Effects of attention and emotion on face processing in the human brain: an event-related fMRI study,” Neuron 30 (3), 829-841; Keil et al., 2005, “Additive effects of emotional content and spatial selective attention on electrocortical facilitation,” Cereb. Cortex 15 (8), 1187-1197; Brosch et al., 2011, “Additive effects of emotional, endogenous, and exogenous attention: behavioral and electrophysiological evidence,” Neuropsychologia 49, 1779-1787). It is further reported that the amygdala also activates to positive or arousing emotional stimuli (and not only negative or threat-related stimuli), based on human imaging studies (see, e.g., Phan et al., 2002, “Functional neuroanatomy of emotion: a meta-analysis of emotion activation studies in PET and fMRI,” NeuroImage 16 (2), 331-348, and Kober et al., 2008, “Functional grouping and cortical-subcortical interactions in emotion: a meta-analysis of neuroimaging studies,” NeuroImage 42 (2), 998-1031) and therefore may potentially induce similar emotional biases (see Pourtois et al.).

Pourtois et al. reports that lesions of the amygdala in humans have been shown to adversely affect neural responses to emotional faces in structurally intact visual cortex (based on fMRI results in Vuilleumier et al., 2004, “Distant influences of amygdala lesion on visual cortical activation during emotional face processing,” Nature Neuroscience, 7 (11), 1271-1278), while patients with temporal lobe sclerosis sparing the amygdala and affecting the hippocampus showed a normal pattern of emotional increases in fusiform cortex. It is further reported that, besides the direct feedback connections from amygdala discussed here, emotional biases could also influence perception and attention via indirect pathways (Vuilleumier, 2005, “How brains beware: neural mechanisms of emotional attention,” Trends in Cognitive Science 9 (12), 585-594; Lim et al., 2009, “Segregating the significant from the mundane on a moment-to-moment basis via direct and indirect amygdala contributions,” Proc. Natl. Acad. Sci. U.S.A. 106 (39), 16841-16846). Data reportedly indicates that, due to the many output projections from the amygdala, emotional processing may have multiple ways to influence in a rapid and powerful manner a variety of cognitive functions at the perception level, attention level, and also motor functions (see Sagaspe et al., 2011, “Fear and stop: a role for the amygdala in motor inhibition by emotional signals,” NeuroImage 55 (4), 1825-1835).

Pourtois et al. also reports that neuroimaging results for different categories of anxiety disorders suggest that each disorder tends to be associated with a distinctive pattern of changes in brain areas overlapping with those involved in emotional attention (see also Etkin et al., 2007, “Functional neuroimaging of anxiety: a meta-analysis of emotional processing in PTSD, social anxiety disorder, and specific phobia,” American Journal Psychiatry 164 (10), 1476-1488).

As another example, Keightley et al., 2003, Neuropsychologia, 41, 585-596, reports the results of an investigation using fMRI of brain regions modulated by cognitive tasks during emotional processing, based on emotional processing tasks on positive and negative faces and pictures (i.e., faces and pictures with differing valences). The article reports that increased activity in the amygdala during processing of faces can depend on factors such as emotional valence and type of task, and may not require that attention be focused on the emotional expression itself or even on the face. It is also reported that activity in the brain regions involved in processing facial expression is modulated by task demands. For example, subjects were required to make an incidental (gender) or explicit (valence) decision about faces portraying neutral, happy or disgusted expressions. Keightley et al reports that activation of left inferior frontal and bilateral occipital-temporal regions is common to all conditions, whereas explicit judgements of disgust were associated with activity in the left amygdala and explicit judgements of happiness were characterized by bilateral orbitofrontal cortex activity. It is reported in Keightley et al. that cognitive processing of a facial expression, such as would be necessary for attaching a verbal label to it, reduces the level of arousal associated with perception of a potentially threatening stimulus such as an angry face.

Gorno-Tempini et al., 2001, “Explicit and incidental facial expression processing: An fMRI study,” NeuroImage 14, 465-73, reports a study where subjects were required to make an incidental (gender) or explicit (valence) decision about faces portraying neutral, happy or disgusted expressions. The fMRI measurements showed that activation of left inferior frontal and bilateral occipital-temporal regions was common to all conditions, whereas explicit judgements of disgust were associated with activity in the left amygdala and explicit judgements of happiness were characterized by bilateral orbitofrontal cortex activity. Hariri et al., 2000, “Modulating emotional responses: effects of a neocortical network on the limbic system,” NeuroReport 11, 43-8. report that matching angry expressions increased activity in the amygdala bilaterally, while labelling expressions was associated with decreased activity in the same regions. They interpreted this finding as evidence that brain activity in limbic regions is modulated by higher brain regions (e.g., pre-frontal cortex) via intellectual processes such as labelling. It may be that cognitive processing of a facial expression, such as would be necessary for attaching a verbal label to it, reduces the level of arousal associated with perception of a potentially threatening stimulus such as an angry face. The results reported in Hariri et al. and Gorno-Tempini et al. shows that the requirement of an individual to make a response to a stimulus under emotional load, such as to make a decision to label the stimulus can result in measurable physiological changes in the individual's neural activity and the regions of the brain activated as compared to if the individual is not required to respond to the stimulus. The faces portraying differing facial expressions (of differing valence) result in differing emotional load. The results reported in Hariri et al. and Gorno-Tempini et al. also shows that the neural activity and regions of the brain activated with the requirement to respond (e.g., label) the stimulus can differ depending on the emotional load evoked by the stimuli. As reported in the various references described herein, changes in neural activity and regions of the brain activated based on the level of emotional load evoked by the stimuli can be manifested in measurable differences in the individual's performance of tasks in the presence of the stimuli.

Keightley et al. also reports that the amygdala and related regions (thalamus, insula, rostral anterior cingulate, ventral and inferior prefrontal cortex) are suggested to form a “primitive” neural system for processing emotional stimuli with biological significance, such as fearful/angry faces, and cognitive tasks demanding increased attention attenuate activity in these brain regions and increase activity in dorsal areas. Keightley et al. also reports that emotional faces trigger the limbic regions in this neural network in an automatic, perhaps pre-attentive fashion, whereas emotional pictures trigger them only when attention is focused on the emotional content. Keightley et al. indicates that these findings are relevant from a clinical perspective in supporting a conclusion that the intricate nature of the interaction between these regions of the brain can be compromised by various mood and cognitive disorders (e.g., depression and Alzheimer's disease, data on these regions can provide insight into the impairments in information processing associated with these mood and cognitive disorders.

In the review article by Vuilleumier, 2005, “How brains beware: neural mechanisms of emotional attention,” TRENDS in Cognitive Sciences, Vol. 9 No. 12, 585-594, it is reported that, under conditions where the deployment of attentional resources is limited, in space or in time, emotional information is prioritized and receives privileged access to an individual's attention and awareness (see also Fox, E., 2002, “Processing of emotional facial expressions: The role of anxiety and awareness,” Cognitive Affective Behavioral Neuroscience 2, 52-63, and Vuilleumier, et al., 2001, “Emotional facial expressions capture attention,” Neurology 56, 153-158). It is also reported that this advantage is produced by various emotional signals, including faces, words, complex scenes, or aversively conditioned stimuli, as well as feared objects in people with specific phobias (e.g., snakes, spiders). The review article indicates that emotional biases appear stronger with ‘biologically prepared’ stimuli (e.g. faces) and with negative or threat-related emotions (e.g. fear or anger), while pleasant and arousing stimuli can also have similar effects, suggesting that arousal value rather than just valence of the stimulus (negative vs positive) can play a crucial role (e.g., Anderson, A. K., 2005, “Affective influences on the attentional dynamics supporting awareness,” Journal of Experimental Psychology: General 134, 258-281).

The Vuilleumier 2005 review article also reports that neuroimaging and neurophysiology results demonstrate a relative boosting of the neural representation of task-relevant (i.e. attended) information, at the expense of competing and irrelevant (i.e. unattended) stimuli, indicating that neural activity produced by visual stimuli is either enhanced or suppressed depending on whether the stimulus is attended or not, at both early stages and later stages of processing (e.g., temporal cortex).

The Vuilleumier 2005 review article also reports on reports of physiological measurements indicating responses of an individual (including neural activity) implicated with differing emotional load. For example, neuroimaging studies using PET and fMRI show enhanced responses to emotional stimuli relative to neutral stimuli—including angry or fearful faces, threat words, aversive pictures, and fear-conditioned stimuli. (See also Lane et al., 1999, “Common effects of emotional valence, arousal, and attention on neural activation during visual processing of pictures,” Neuropsychologia 37, 989-997; Morris et al., 1998, “A neuromodulatory role for the human amygdala in processing emotional facial expressions,” Brain 121, 47-57; Vuilleumier et al., 2001, “Effects of attention and emotion on face processing in the human brain: An event-related fMRI study,” Neuron 30, 829-841; and Sabatinelli et al., 2005, “Parallel amygdala and inferotemporal activation reflect emotional intensity and fear relevance,” Neuroimage 24, 1265-1270). Enhanced responses to emotional visual stimuli are reported in the auditory cortex for emotional sounds or voices. (See, e.g., Mitchell et al., 2003, “The neural response to emotional prosody, as revealed by functional magnetic resonance imaging,” Neuropsychologia 41, 1410-1421; Sander et al., 2001, “Auditory perception of laughing and crying activates human amygdala regardless of attentional state,” Brain Res. Cogn. Brain Res. 12, 181-198; and Grandjean et al., 2005, “The voices of wrath: brain responses to angry prosody in meaningless speech,” Nature Neuroscience 8, 145-146). The results of EEG and MEG studies also reported to show amplified responses to emotional visual events, involving early sensory components (e.g., at 120-150 ms), as well as later cognitive components (e.g. after 300-400 ms). (See, e.g., Eimer et al., 2007, “Event-related potential correlates of emotional face processing,” Neuropsychologia 45(1), 15-31; Pourtois et al., 2005, “Enhanced extrastriate visual response to bandpass spatial frequency filtered fearful faces: Time course and topographic evoked-potentials mapping,” Hum. Brain Ma26, 65-79; Batty et al., 2003, “Early processing of the six basic facial emotional expressions,” Brain Res. Cogn. Brain Res. 17, 613-620; Carretie et al., 2004, “Automatic attention to emotional stimuli: neural correlates,” Hum. Brain Ma 22, 290-299, Krolak-Salmon et al., 2001, “Processing of facial emotional expression: spatio-temporal data as assessed by scalp event-related potentials,” European Journal of Neuroscience 13, 987-994; Schupp et al., 2003, “Attention and emotion: an ERP analysis of facilitated emotional stimulus processing,” Neuroreport 14, 1107-1110). These increased sensory responses can arise even when an individual is not required to pay attention to the emotional meaning of a stimulus.

The Vuilleumier 2005 review article also reports that stronger neuronal activation can render emotional stimuli more resistant to the suppressive interference caused by distractors. The review article concludes that, consistent with models of attention based on biased competition, the boosting of responses can generate a more robust and sustained representation of emotional stimuli within the sensory pathways, yielding a stronger weight in the competition for attentional resources and prioritized access to awareness, relative to the weaker signals generated by any competing neutral stimuli (resulting in emotional events being more swiftly discerned, or more difficult to ignore, than ordinary neutral events).

The emotional load evoked by a stimulus can vary depending on the state of an individual, including based on the individual's cognitive condition, disease, or executive function disorder. Measurements of the individual's performance under emotional load can provide insight into the individual's status relative to a cognitive condition, disease, or executive function disorder, including the likelihood of onset and/or stage of progression of the cognitive condition, disease, or executive function disorder. For example, Breitenstein et al., 1998, “Emotional processing following cortical and subcortical brain damage,” Behavioural Neurology 11, 29-42, reports the results of PET and fMRI studies in normal control subjects, which show that fearful stimuli activated the amygdala and disgust stimuli the anterior insular cortex. (See also Morris et al., 1996, “A differential neural response in the human amygdala to fearful and happy facial expressions, Nature 383, 812-815; and Phillips et al., 1997, “A specific neural substrate for perceiving facial expressions of disgust,” Nature 389, 495-498.) Breitenstein et al. 1998 also reports that especially severe deficits can occur in the recognition of facial and vocal expressions of disgust (and to a lesser extent fear) in individuals with Huntington's disease as well as Huntington's disease gene carriers. (See, e.g., Gray et al., 1997, “Impaired recognition of disgust in Huntington's disease gene carriers,” Brain 120 (1997), 2029-2038; and Sprengelmeyer et al., 1996, “Loss of disgust—Perception of faces and emotions in Huntington's disease,” Brain 119, 1647-1665.) Breitenstein et al. 1998 also reports that neocortical degeneration in individuals with Huntington's disease is widespread (involving both the basal ganglia as well as posterior cortex regions). It is reported that the basal ganglia plays a role in emotion processing (see, e.g., Cancelliere et al., 1990, “Lesion localization in acquired deficits of emotional expression and comprehension,” Brain and Cognition 13, 133-147). Data that can be provided on Huntington's disease gene carriers (i.e., clinically pre-symptomatic individuals) can be of interest with respect to neural substrates of emotion, since basal ganglia structures (caudate nucleus) are affected earliest by the neurodegeneration of Huntington's disease. Studies also describe prosodic and facial comprehension disorders in individuals with Parkinson's disease, a neurological condition with primarily dysregulation of the basal ganglia, where individuals exhibited reduced performance in identification of affective prosody and facial expressions in individuals with Parkinson's disease (see, e.g., Scott et al., 1984, “Evidence for an apparent sensory speech disorder in Parkinson's disease,” Journal of Neurology, Neurosurgery, and Psychiatry 47, 840-843).

The foregoing non-limiting examples of physiological measurement data, behavioral data, and other cognitive data, show that the responses of an individual to tasks can differ based on emotional load (including the presence or absence of emotional or affective stimuli). Furthermore, the foregoing examples indicate that the degree to which an individual is affected by an evocative element, and the degree to which the performance of the individual at a task is affected in the presence of the evocative element, is dependent on the degree to which the individual exhibits a form of emotional or affective bias. As described herein, the differences in the individual's performance may be quantifiably sensed and measured based on the performance of the individual at cognitive tasks versus stimuli with evocative elements (e.g., emotional or affective elements). The reported physiological measurement data, behavioral data, and other cognitive data, also show that the emotional load evoked by a stimulus can vary depending on the state of an individual, including based on the individual's cognitive condition, disease state, or presence or absence of executive function disorder. As described herein, measurements of the differences in the individual's performance at cognitive tasks versus stimuli with evocative elements can provide quantifiable insight into the likelihood of onset and/or stage of progression of a cognitive condition, disease, and/or executive function disorder, in the individual, such as but not limited to, social anxiety, depression, bipolar disorder, major depressive disorder, post-traumatic stress disorder, schizophrenia, autism spectrum disorder, attention deficit hyperactivity disorder, dementia, Parkinson's disease, Huntington's disease, or other neurodegenerative condition, Alzheimer's disease, or multiple sclerosis.

The effects of interference processing on the cognitive control abilities of individuals has been reported. See, e.g., A. Anguera, Nature 501, p. 97 (Sep. 5, 2013) (the “Nature article”). See, also, U.S. Publication No. 20140370479A1 (U.S. application Ser. No. 13/879,589), filed on Nov. 10, 2011, which is incorporated herein by reference. Some of those cognitive abilities include cognitive control abilities in the areas of attention (selectivity, sustainability, etc.), working memory (capacity and the quality of information maintenance in working memory) and goal management (ability to effectively parallel process two attention-demanding tasks or to switch tasks). As an example, children diagnosed with ADHD (attention deficit hyperactivity disorder) exhibit difficulties in sustaining attention. Attention selectivity was found to depend on neural processes involved in ignoring goal-irrelevant information and on processes that facilitate the focus on goal-relevant information. The publications report neural data showing that when two objects are simultaneously placed in view, focusing attention on one can pull visual processing resources away from the other. Studies were also reported showing that memory depended more on effectively ignoring distractions, and the ability to maintain information in mind is vulnerable to interference by both distraction and interruption. Interference by distraction can be, e.g., an interference that is a non-target, that distracts the individual's attention from the primary task, but that the instructions indicate the individual is not to respond to. Interference by interruption/interruptor can be, e.g., an interference that is a target or two or more targets, that also distracts the individual's attention from the primary task, but that the instructions indicate the individual is to respond to (e.g., for a single target) or choose between/among (e.g., a forced-choose situation where the individual decides between differing degrees of a feature).

There were also fMRI results reported showing that diminished memory recall in the presence of a distraction can be associated with a disruption of a neural network involving the prefrontal cortex, the visual cortex, and the hippocampus (involved in memory consolidation). Prefrontal cortex networks (which play a role in selective attention) can be vulnerable to disruption by distraction. The publications also report that goal management, which requires cognitive control in the areas of working memory or selective attention, can be impacted by a secondary goal that also demands cognitive control. The publications also reported data indicating beneficial effects of interference processing as an intervention with effects on an individual's cognitive abilities, including to diminish the detrimental effects of distractions and interruptions. The publications described cost measures that can be computed (including an interference cost) to quantify the individual's performance, including to assess single-tasking or multitasking performance.

An example cost measure disclosed in the publications is the percentage change in an individual's performance at a single-tasking task as compared to a multi-tasking task, such that greater cost (that is, a more negative percentage cost) indicates increased interference when an individual is engaged in single-tasking vs multi-tasking. The publications describe an interference cost determined as the difference between an individual's performance on a task in isolation versus a task with one or more interference applied, where the interference cost provide an assessment of the individual's susceptibility to interference.

The tangible benefits of computer-implemented interference processing are also reported. For example, the Nature paper states that multi-tasking performance assessed using computer-implemented interference processing was able to quantify a linear age-related decline in performance in adults from 20 to 79 years of age. The Nature paper also reports that older adults (60 to 85 years old) who interacted with an adaptive form of the computer-implemented interference processing exhibited reduced multitasking costs, with the gains persisting for six (6) months. The Nature paper also reported that age-related deficits in neural signatures of cognitive control, as measured with electroencephalography, were remediated by the multitasking training (using the computer-implemented interference processing), with enhanced midline frontal theta power and frontal-posterior theta coherence. Interacting with the computer-implemented interference processing resulted in performance benefits that extended to untrained cognitive control abilities (enhanced sustained attention and working memory), with an increase in midline frontal theta power predicting a boost in sustained attention and preservation of multitasking improvement six (6) months later.

The example systems, methods, and apparatus according to the principles herein are configured to classify an individual as to cognitive abilities and/or to enhance those cognitive abilities based on implementation of interference processing using a computerized cognitive platform. The example systems, methods, and apparatus are configured to implement a form of multi-tasking using the capabilities of a programmed computing device, where an individual is required to perform a task and an interference substantially simultaneously, where the task and/or the interference includes an evocative element, and the individual is required to respond to the evocative element. The sensing and measurement capabilities of the computing device are configured to collect data indicative of the physical actions taken by the individual during the response execution time to respond to the task at substantially the same time as the computing device collects the data indicative of the physical actions taken by the individual to respond to the evocative element. The capabilities of the computing devices and programmed processing units to render the task and/or the interference in real time to a user interface, and to measure the data indicative of the individual's responses to the task and/or the interference and the evocative element in real time and substantially simultaneously can provide quantifiable measures of an individual's cognitive capabilities under emotional load, to rapidly switch to and from different tasks and interferences under emotional load, or to perform multiple, different, tasks or interferences in a row under emotional load (including for single-tasking, where the individual is required to perform a single type of task for a set period of time).

In any example herein, the task and/or interference includes a response deadline, such that the user interface imposes a limited time period for receiving at least one type of response from the individual interacting with the apparatus or computing device. For example, the period of time that an individual is required to interact with a computing device or other apparatus to perform a task and/or an interference can be a predetermined amount of time, such as but not limited to about 30 seconds, about 1 minute, about 4 minutes, about 7 minutes, about 10 minutes, or greater than 10 minutes.

The example systems, methods, and apparatus can be configured to implement a form of multi-tasking to provide measures of the individual's capabilities in deciding whether to perform one action instead of another and to activate the rules of the current task in the presence of an interference such that the interference diverts the individual's attention from the task, as a measure of an individual's cognitive abilities in executive function control.

The example systems, methods, and apparatus can be configured to implement a form of single-tasking, where measures of the individual's performance at interacting with a single type of task (i.e., with no interference) for a set period of time (such as but not limited to navigation task only or a target discriminating task only) can also be used to provide measure of an individual's cognitive abilities.

The example systems, methods, and apparatus can be configured to implement sessions that involve differing sequences and combinations of single-tasking and multi-tasking trials. In a first example implementation, a session can include a first single-tasking trial (with a first type of task), a second single-tasking trial (with a second type of task), and a multi-tasking trial (a primary task rendered with an interference). In a second example implementation, a session can include two or more multi-tasking trials (a primary task rendered with an interference). In a third example implementation, a session can include two or more single-tasking trials (all based on the same type of tasks or at least one being based on a different type of task).

The performance can be further analyzed to compare the effects of two different types of interference (e.g. distraction or interruptor) on the performances of the various tasks. Some comparisons can include performance without interference, performance with distraction, and performance with interruption. The cost of each type of interference (e.g.distraction cost and interruptor/multi-tasking cost) on the performance level of a task is analyzed and reported to the individual.

In any example herein, the interference can a secondary task that includes a stimulus that is either a non-target (as a distraction) or a target (as an interruptor), or a stimulus that is differing types of targets (e.g., differing degrees of a facial expression or other characteristic/feature difference).

Based on the capability of a programmed processing unit to control the effecting of multiple separate sources (including sensors and other measurement components) and the receiving of data selectively from these multiple different sources at substantially simultaneously (i.e., at roughly the same time or within a short time interval) and in real-time, the example systems, methods, and apparatus herein can be used to collect quantitative measures of the responses form an individual to the task and/or interference under emotional load, which could not be achieved using normal human capabilities. As a result, the example systems, methods, and apparatus herein can be configured to implement a programmed processing unit to render the interference substantially simultaneously with the task over certain time periods.

In some example implementations, the example systems, methods, and apparatus herein also can be configured to receive the data indicative of the measure of the degree and type of the individual's response to the task substantially simultaneously as the data indicative of the measure of the degree and type of the individual's response to the interference is collected (whether the interference includes a target or a non-target). In some examples, the example systems, methods, and apparatus are configured to perform the analysis by applying scoring or weighting factors to the measured data indicative of the individual's response to a non-target that differ from the scoring or weighting factors applied to the measured data indicative of the individual's response to a target, in order to compute a cost measure (including an interference cost).

In an example systems, methods, and apparatus herein, the cost measure can be computed based on the difference in measures of the performance of the individual at one or more tasks in the absence of interference as compared to the measures of the performance of the individual at the one or more tasks in the presence of interference, where the one or more tasks and/or the interference includes one or more evocative elements rendered in modes. As described herein, the requirement of the individual to interact with (and provide a response to) the evocative element(s) can introduce emotional load that quantifiably affects the individuals capability at performing the task(s) and/or interference due to the requirement for emotional processing to respond to the evocative element. In an example, the interference cost computed based on the data collected herein can provide a quantifiable assessment of the individual's susceptibility to interference under emotional load. The determination the difference between an individual's performance on a task in isolation versus a task in the presence of one or more interference (the task and/or interference including the evocative element) provides an interference cost metric that can be used to assess and classify cognitive capabilities of the individual under emotional load. The interference cost computed based on the individuals performance of tasks and/or interference performed under emotional load can also provide a quantifiable measure of the individual's cognitive condition, disease state, or presence or stage of an executive function disorder, such as but not limited to, social anxiety, depression, bipolar disorder, major depressive disorder, post-traumatic stress disorder, schizophrenia, autism spectrum disorder, attention deficit hyperactivity disorder, dementia, Parkinson's disease, Huntington's disease, or other neurodegenerative condition, Alzheimer's disease, or multiple sclerosis.

The example systems, methods, and apparatus herein can be configured to perform the analysis of the individual's susceptibility to interference under emotional load (including as a cost measure such as the interference cost), as a reiterating, cyclical process. For example, where an individual is determined to have minimized interference cost for a given task and/or interference under emotional load, the example systems, methods, and apparatus can be configured to require the individual to perform a more challenging task and/or interference under emotional load (i.e., having a higher difficulty level) until the individual's performance metric indicates a minimized interference cost in that given condition, at which point example systems, methods, and apparatus can be configured to present the individual with an even more challenging task and/or interference under emotional load until the individual's performance metric once again indicates a minimized interference cost for that condition. This can be repeated any number of times until a desired end-point of the individual's performance is obtained.

As a non-limiting example, the interference cost can be computed based on measurements of the individual's performance at a single-tasking task (without an interference) as compared to a multi-tasking task (with interference), to provide an assessment. For example, an individual's performance at a multi-tasking task (e.g., targeting task with interference) can be compared to their performance at a single-tasking targeting task without interference to provide the interference cost.

Example systems, apparatus and methods herein are configured to analyze data indicative of the degree to which an individual is affected by an evocative element, and/or the degree to which the performance of the individual at a task is affected in the presence of the evocative element, to provide performance metric including a quantified indicator of cognitive abilities of the individual under emotional load. The performance metric can be used as an indicator of the degree to which the individual exhibits a form of emotional or affective bias.

In some example implementations, the example systems, methods, and apparatus herein also can be configured to selectively receive data indicative of the measure of the degree and type of the individual's response to an interference that includes a target stimulus (i.e., an interruptor) substantially simultaneously (i.e., at substantially the same time) as the data indicative of the measure of the degree and type of the individual's response to the task is collected and to selectively not collect the measure of the degree and type of the individual's response to an interference that includes a non-target stimulus (i.e., a distraction) substantially simultaneously (i.e., at substantially the same time) as the data indicative of the measure of the degree and type of the individual's response to the task is collected. That is, the example systems, methods, and apparatus are configured to discriminate between the windows of response of the individual to the target versus non-target by selectively controlling the state of the sensing/measurement components for measuring the response either temporally and/or spatially. This can be achieved by selectively activating or de-activating sensing/measurement components based on the presentation of a target or non-target, or by receiving the data measured for the individual's response to a target and selectively not receiving (e.g., disregarding, denying, or rejecting) the data measured for the individual's response to a non-target.

As described herein, using the example systems, methods, and apparatus herein can be implemented to provide a measure of the cognitive abilities of an individual in the area of attention, including based on capabilities for sustainability of attention over time, selectivity of attention, and reduction of attention deficit. Other areas of an individual's cognitive abilities that can be measured using the example systems, methods, and apparatus herein include affective bias, mood, level of cognitive bias, impulsivity, inhibition, perceptive abilities, reaction and other motor functions, visual acuity, long-term memory, working memory, short-term memory, logic, and decision-making.

As described herein, using the example systems, methods, and apparatus herein can be implemented to adapt the tasks and/or interference (at least one including an evocative element) from one user session to another (or even from one user trial to another) to enhance the cognitive skills of an individual under emotional load based on the science of brain plasticity. Adaptivity is a beneficial design element for any effective plasticity-harnessing tool. In example systems, methods, and apparatus, the processing unit is configured to control parameters of the tasks and/or interference, such as but not limited to the timing, positioning, and nature of the stimuli, so that the physical actions of the individual can be recorded during the interaction(s). As described hereinabove, the individual's physical actions are affected by their neural activity during the interactions with the computing device to perform single-tasking and multi-tasking tasks. The science of interference processing shows (based on the results from physiological and behavioral measurements) that the aspect of adaptivity can result in changes in the brain of an individual in response to the training from multiple sessions (or trials) based on neuroplasticity, thereby enhancing the cognitive skills of the individual. The example systems, methods, and apparatus are configured to implement tasks and/or interference with at least one evocative element, where the individual performs the interference processing under emotional load. As supported in the published research results described hereinabove, the effect on an individual of performing tasks under emotional load can tap into novel aspects of cognitive training to enhance the cognitive abilities of the individual.

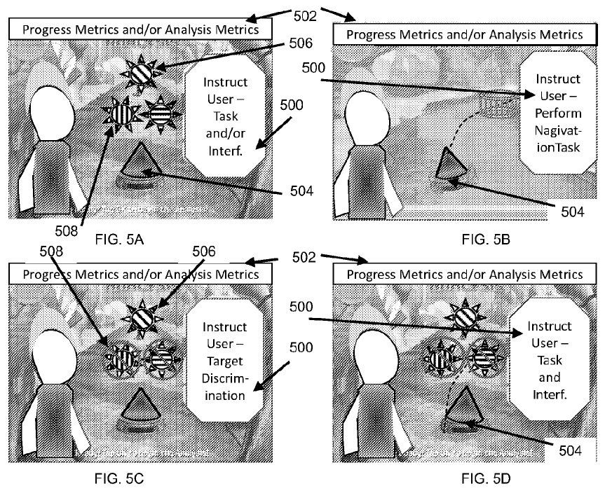

FIGS. 5A-9P show non-limiting example user interfaces that can be rendered using example systems, methods, and apparatus herein to render the tasks and/or interferences (either or both with evocative element) for user interactions. The non-limiting example user interfaces of FIGS. 5A-9P also can be used for one or more of: to display instructions to the individual for performing the tasks and/or interferences, interact with the evocative element, to collect the data indicative of the individual's responses to the tasks and/or the interferences and the evocative element, to show progress metrics, and to provide the analysis metrics.

FIGS. 5A-5D show non-limiting example user interfaces rendered using example systems, methods, and apparatus herein. As shown in FIGS. 5A-5B, an example programmed processing unit can be used to render to the user interfaces (including graphical user interfaces) display features 500 for displaying instructions to the individual for performing the tasks and/or interferences and to interact with the evocative element, and metric features 502 to show status indicators from progress metrics and/or results from application of analytics to the data collected from the individual's interactions (including the responses to tasks/interferences) to provide the analysis metrics. In any example systems, methods, and apparatus herein, the classifier can be used to provide the analysis metrics provided as a response output. In any example systems, methods, and apparatus herein, the data collected from the user interactions can be used as input to train the classifier. As shown in FIGS. 5A-5B, an example programmed processing unit also may be used to render to the user interfaces (including graphical user interfaces) an avatar or other processor-rendered guide 504 that an individual is required to control (such as but not limited to navigate a path or other environment in a visuo-motor task, and/or to select an object in a target discrimination task). In an example, the evocative element may be includes as a component of the visuo-motor task (e.g., as a milestone object along the math) or as a component of the target discrimination task, e.g., where a specific type of evocative element (such as but not limited to an angry or happy face, loud or angry voice or a threat or fear-inducing word) is the target, and other types of the evocative element are not (such as but not limited to a neutral face, a happy voice, or a neutral word). As shown in FIG. 5B, the display features 500 can be used to instruct the individual what is expected to perform a navigation task while the user interface depicts (using the dashed line) the type of movement of the avatar or other processor-rendered guide 504 required for performing the navigation task. In an example, the navigation task may include milestone objects (possibly including evocative elements rendered in modes) that the individual is required to steer an avatar to cross or avoid, in order to determine the scoring. As shown in FIG. 5C, the display features 500 can be used to instruct the individual what is expected to perform a target discrimination task while the user interface depicts the type of object(s) 506 and 508 that may be rendered to the user interface, with one type of object 506 (possibly including a target evocative element) designated as a target while the other type of object 508 that may be rendered to the user interface is designated as a non-target (possibly including a non-target evocative element), e.g., by being crossed out in this example. As shown in FIG. 5D, the display features 500 can be used to instruct the individual what is expected to perform both a navigation task as a primary task and a target discrimination as a secondary task (i.e., an interference) while the user interface depicts (using the dashed line) the type of movement of the avatar or other processor-rendered guide 504 required for performing the navigation task, and the user interface renders the object type designated as a target object 506 and the object type designated as a non-target object 508.

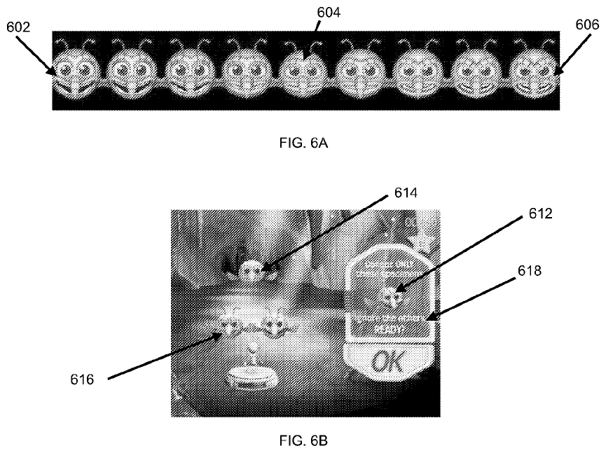

FIGS. 6A-6B show examples of the evocative elements (targets or non-targets) that can be rendered to an example user interface, according to the principles herein. FIG. 6A shows an example of the evocative elements rendered as differing types of facial expressions, including facial expressions with positive valence (happy) and facial expressions negative valence (angry). For example, the evocative elements can be rendered as a face with a happy expression 602, a neutral expression 604, or an angry expression 606. FIG. 6A also shows modulations of the facial expression of the evocative element, showing differing degrees of the facial expression from the very happy face 602 (highest degree) with gradual reduction of the degree of happiness down to the neutral face 604, and also showing differing degrees of the facial expression from the very angry face 606 (highest degree) with gradual reduction of the degree of anger down to the neutral face 604, with each potentially evoking differing levels of emotional response in an individual. FIG. 6B shows an example user interface with evocative elements rendered as differing types of facial expressions (happy 610, neutral 614, angry 616). FIG. 6B also shows an example display feature 618 for displaying instructions to the individual for performing the tasks and/or interferences and to interact with the evocative element. In the non-limiting example of FIG. 6B, the display feature 618 can be used to instruct the individual what is expected to perform a target discrimination task, with an indication of the type of response required for the evocative element (in this example, recognize and target the happy face 612.

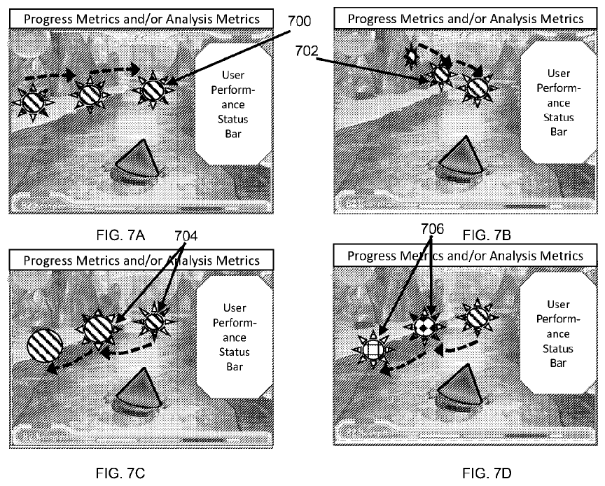

FIGS. 7A-7D show examples of the features of object(s) (targets or non-targets) that can be rendered as time-varying characteristics to an example user interface, according to the principles herein. FIG. 7A shows an example where the modification to the time-varying characteristics of an aspect of the object 700 rendered to the user interface is a dynamic change in position and/or speed of the object 700 relative to environment rendered in the graphical user interface. FIG. 7B shows an example where the modification to the time-varying characteristics of an aspect of the object 702 rendered to the user interface is a dynamic change in size and/or direction of trajectory/motion, and/or orientation of the object 702 relative to the environment rendered in the graphical user interface. FIG. 7C shows an example where the modification to the time-varying characteristics of an aspect of the object 704 rendered to the user interface is a dynamic change in shape or other type of the object 704 relative to the environment rendered in the graphical user interface. In this non-limiting example, the time-varying characteristic of object 704 is effected using morphing from a first type of object (a star object) to a second type of object (a round object). In another non-limiting example, the time-varying characteristic of object 704 is effected by rendering a blendshape as a proportionate combination of a first type of object and a second type of object. FIG. 7C shows an example where the modification to the time-varying characteristics of an aspect of the object 704 rendered to the user interface is a dynamic change in shape or other type of the object 704 rendered in the graphical user interface (in this non-limiting example, from a star object to a round object). FIG. 7D shows an example where the modification to the time-varying characteristics of an aspect of the object 706 rendered to the user interface is a dynamic change in a non-evocative feature (such as but not limited to the pattern, or color, or visual feature) of the object 706 relative to environment rendered in the graphical user interface (in this non-limiting example, from a star object having a first pattern to a round object having a second pattern). In another non-limiting example, the time-varying characteristic of object can be a rate of change of a facial expression depicted on or relative to the object. In any example herein, the foregoing time-varying characteristic can be applied to an object including the evocative element to modify an emotional load of the individual's interaction with the apparatus (e.g., computing device or cognitive platform).

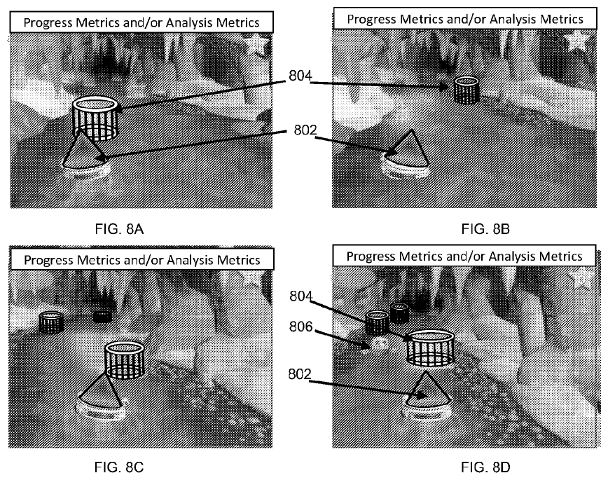

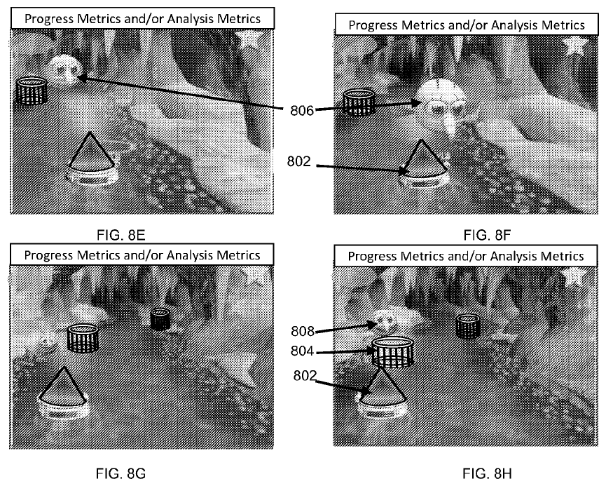

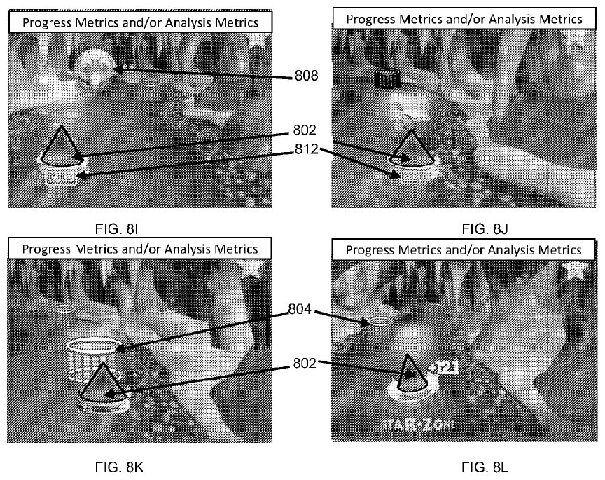

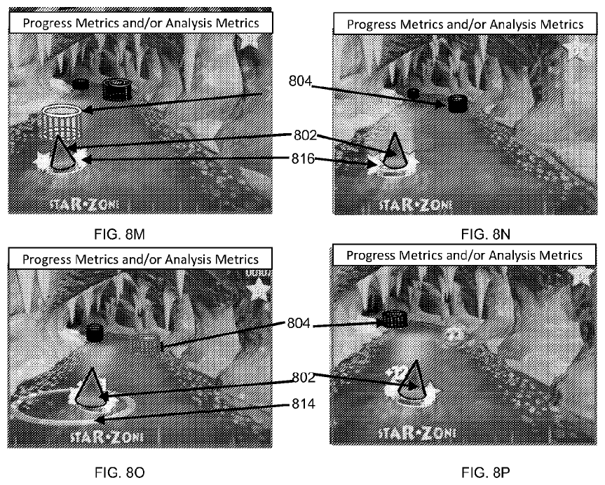

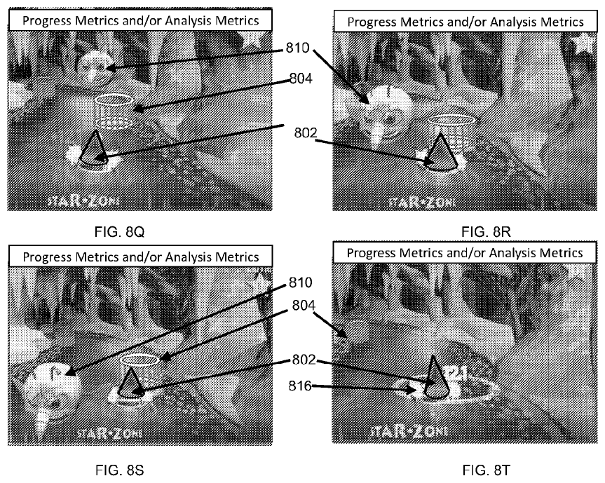

FIGS. 8A-8T show a non-limiting example of the dynamics of tasks and interferences that can be rendered at user interfaces, according to the principles herein. In this example, the task is a visuo-motor navigation task, and the interference is target discrimination (as a secondary task). The evocative element is rendered faces with differing facial expressions, and the evocative element is a part of the interference. The example system is programmed to instruct the individual to perform the visuo-motor task and target discrimination (with identification of a specific facial expression as the response to the evocative element). As shown in FIGS. 8A-8T, the individual is required to perform the navigation task by controlling the motion of the avatar 802 along a path that coincides with the milestone objects 804. FIGS. 8A-8T show a non-limiting example implementation where the individual is expected to actuate an apparatus or computing device (or other sensing device) to cause the avatar 802 to coincide with the milestone object 804 as the response in the navigation task, with scoring based on the success of the individual at crossing paths with (e.g., hitting) the milestone objects 804. In another example, the individual is expected to actuate an apparatus or computing device (or other sensing device) to cause the avatar 802 to miss the milestone object 804, with scoring based on the success of the individual at avoiding the milestone objects 804. FIGS. 8A-8T also show the dynamics of a non-target object 806 having an first type of evocative element (a neutral facial expression), where the time-varying characteristic is the trajectory of motion of the object. FIGS. 8A-8T also show the dynamics of a target object 808 having a second type of evocative element (a happy facial expression), where the time-varying characteristic is the trajectory of motion of the object. FIGS. 8A-8T also show the dynamics of another non-target object 810 having a third type of evocative element (an angry facial expression), where the time-varying characteristic is the trajectory of motion of the object.

In the example of FIGS. 8A-8T, the processing unit of the example system, method, and apparatus is configured to receive data indicative of the individual's physical actions to cause the avatar 802 to navigate the path. For example, the individual may be required to perform physical actions to “steer” the avatar, e.g., by changing the rotational orientation or otherwise moving a computing device. Such action can cause a gyroscope or accelerometer or other motion or position sensor device to detect the movement, thereby providing measurement data indicative of the individual's degree of success in performing the navigation task.

In the example of FIGS. 8A-8T, the processing unit of the example system, method, and apparatus is configured to receive data indicative of the individual's physical actions to perform the target discrimination and to identify a specified evocative element (i.e., a specified facial expression). For example, the individual may be instructed prior to a trial or other session to tap, or make other physical indication, in response to display of a target object having the specified evocative element 808, and not to tap to make the physical indication in response to display of a non-target object 806 or 810 (based on the type of the evocative element). In FIGS. 8A-8C and 8E-8H, the target discrimination acts as an interference (i.e., a secondary task) to the primary navigation task, in an interference processing multi-tasking implementation. As described hereinabove, the example systems, methods, and apparatus can cause the processing unit to render a display feature (e.g., display feature 500) to display the instructions to the individual as to the expected performance (i.e., which evocative element to respond to, and how to perform the target discrimination and navigation tasks). As also described hereinabove, the processing unit of the example system, method, and apparatus can be configured to (i) receive the data indicative of the measure of the degree and type of the individual's response to the primary task substantially simultaneously as the data indicative of the measure of the individual's response to the evocative element is collected (for a specified evocative element), or (i) to selectively receive data indicative of the measure of the individual's response to the specified evocative element as a target stimulus (i.e., an interruptor) substantially simultaneously (i.e., at substantially the same time) as the data indicative of the measure of the degree and type of the individual's response to the task is collected and to selectively not collect the measure of the individual's response to the non-specified evocative element a non-target stimulus (i.e., a distraction) substantially simultaneously (i.e., at substantially the same time) as the data indicative of the measure of the degree and type of the individual's response to the task is collected.

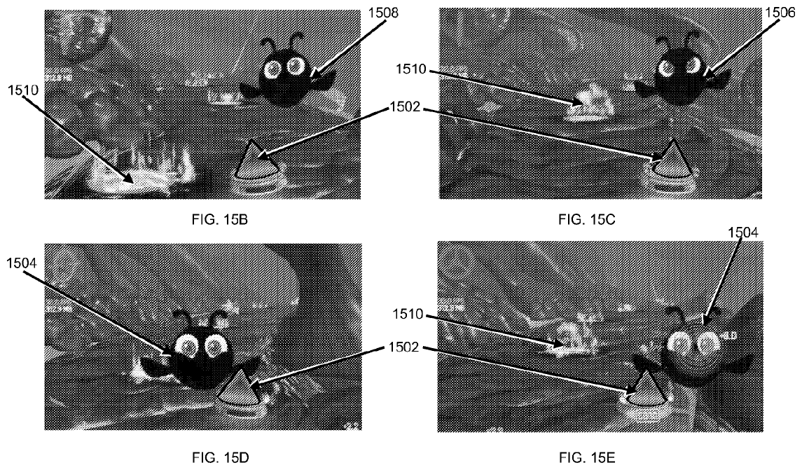

In FIGS. 8A-8T, a feature 812 including the word “GOOD” is rendered near the avatar 802 to signal to the individual that analysis of the data indicative of the individual's responses to the navigation task and target discrimination interference including the evocative element indicate satisfactory performance. FIG. 15V shows an example of a change in the type of rewards presented to the individual as another indication of satisfactory performance, including at least one modification to the avatar 802 to symbolize excitement, such as but not limited to the rings 814 or other active element and/or showing jet booster elements 816 that become star-shaped (and reward graphics such as but not limited to the “STAR-ZONE” graphic). Many other types of reward elements can be used, and the rate and type of reward elements displayed can be changed and modulated as a time-varying element

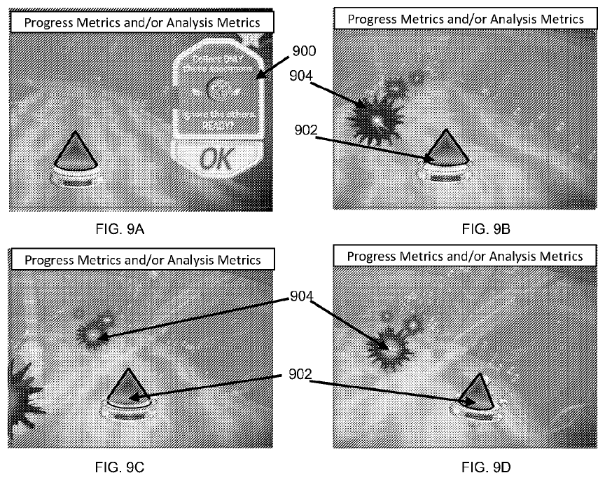

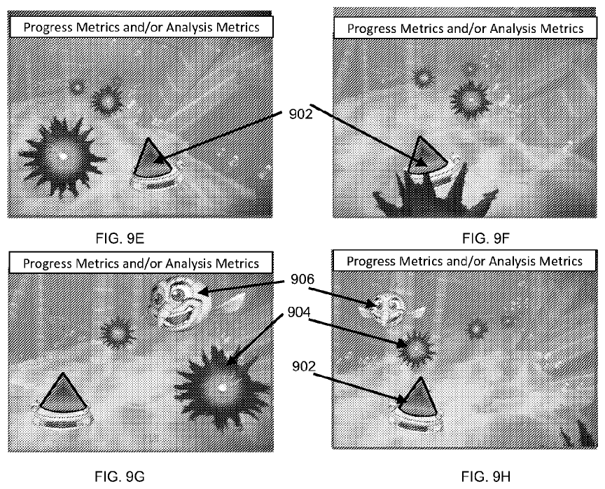

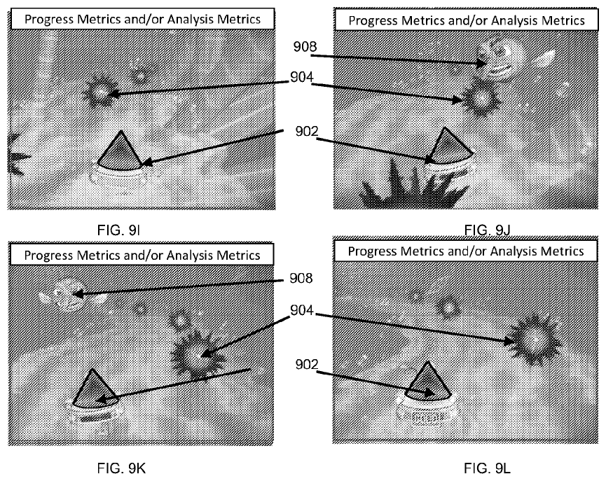

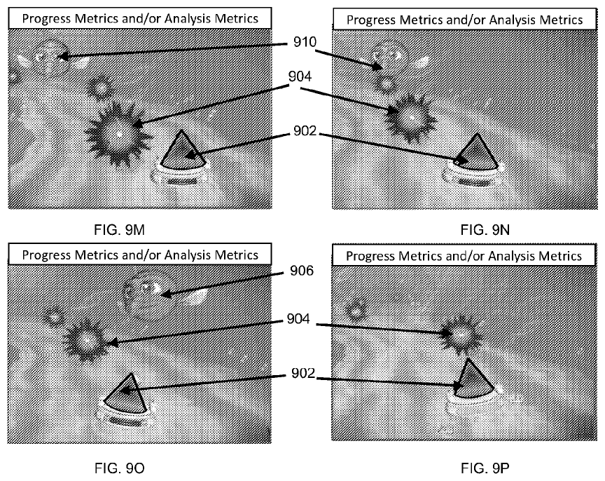

FIGS. 9A-9P show a non-limiting example of the dynamics of tasks and interferences that can be rendered at user interfaces, according to the principles herein. In this example, the task is a visuo-motor navigation task, and the interference is target discrimination (as a secondary task). The evocative element is rendered faces with differing facial expressions, and the evocative element is a part of the interference. FIG. 9A shows an example display feature 900 that can be rendered to instruct the individual to perform the visuo-motor task and target discrimination (with identification of a specific facial expression as the response to the evocative element). As shown in FIGS. 9A-9P, the individual is required to perform the navigation task by controlling the motion of the avatar 902 along a path that avoids (i.e., does not coincides with) the milestone objects 904. FIGS. 9A-9P show a non-limiting example implementation where the individual is expected to actuate an apparatus or computing device (or other sensing device) to cause the avatar 902 to avoid the milestone object 904 as the response in the navigation task, with scoring based on the success of the individual at not crossing paths with (e.g., not hitting) the milestone objects 904. FIGS. 9A-9P also show the dynamics of a non-target object 906 having a first type of evocative element (a happy facial expression), where the time-varying characteristic is the trajectory of motion of the object. FIGS. 9A-9P also show the dynamics of a target object 908 having a second type of evocative element (an angry facial expression), where the time-varying characteristic is the trajectory of motion of the object. FIGS. 9A-9P also show the dynamics of another non-target object 910 having a third type of evocative element (an angry facial expression), where the time-varying characteristic is the trajectory of motion of the object.

In the example of FIGS. 9A-9P, the processing unit of the example system, method, and apparatus is configured to receive data indicative of the individual's physical actions to cause the avatar 902 to navigate the path. For example, the individual may be required to perform physical actions to “steer” the avatar, e.g., by changing the rotational orientation or otherwise moving a computing device. Such action can cause a gyroscope or accelerometer or other motion or position sensor device to detect the movement, thereby providing measurement data indicative of the individual's degree of success in performing the navigation task.

In the example of FIGS. 9A-9P, the processing unit of the example system, method, and apparatus is configured to receive data indicative of the individual's physical actions to perform the target discrimination and to identify a specified evocative element (i.e., a specified facial expression). For example, the individual may be instructed using display feature 900 prior to a trial or other session to tap, or make other physical indication, in response to display of a target object having the specified evocative element 908, and not to tap to make the physical indication in response to display of a non-target object 906 or 910 (based on the type of the evocative element). In FIGS. 9A-9P, the target discrimination acts as an interference (i.e., a secondary task) to the primary navigation task, in an interference processing multi-tasking implementation. As described hereinabove, the example systems, methods, and apparatus can cause the processing unit to render a display feature (e.g., display feature 500) to display the instructions to the individual as to the expected performance (i.e., which evocative element to respond to, and how to perform the target discrimination and navigation tasks). As also described hereinabove, the processing unit of the example system, method, and apparatus can be configured to (i) receive the data indicative of the measure of the degree and type of the individual's response to the primary task substantially simultaneously as the data indicative of the measure of the individual's response to the evocative element is collected (for a specified evocative element), or (i) to selectively receive data indicative of the measure of the individual's response to the specified evocative element as a target stimulus (i.e., an interruptor) substantially simultaneously (i.e., at substantially the same time) as the data indicative of the measure of the degree and type of the individual's response to the task is collected and to selectively not collect the measure of the individual's response to the non-specified evocative element a non-target stimulus (i.e., a distraction) substantially simultaneously (i.e., at substantially the same time) as the data indicative of the measure of the degree and type of the individual's response to the task is collected.

In various examples, the degree of non-linearity of the accumulation of belief for an individual's decision making (i.e., as to whether to execute a response) can be modulated based on adjusting the time-varying characteristics of the task and/or interference. As a non-limiting example, where the time-varying characteristic is a trajectory, speed, orientation, or size of the object (target or non-target), the amount of information available to an individual to develop a belief (in order to make decision as to whether to execute a response) can be made smaller initially, e.g., where the object caused to be more difficult to discriminate by being rendered as farther away or smaller, and can be made to increase at differing rates (nonlinearly) depending on how quickly more information is made available to the individual to develop belief (e.g., as the object is rendered to appear to get larger, change orientation, move slower, or move closer in the environment). Other non-limiting example time-varying characteristics of the task and/or interference that can be adjusted to modulate the degree of non-linearity of the accumulation of belief include one or more of a rate of change of a facial expression, at least one color of an object, the type of the object, a rate of morphing of a first type of object to change to a second type of object, and a blendshape of evocative elements (e.g., a blendshape of facial expressions).

The data indicative of the individual's response to the task and the response of the individual to the at least one evocative element is used to compute at least one performance metric comprising at least one quantified indicator of cognitive abilities of the individual under emotional load. In a non-limiting example, the performance metric can include the computed interference cost under emotional load.

The difficulty levels (including the difficulty of the task and/or interference, and of the evocative element) of a subsequent session can be set based on the performance metric computed for the individual's performance from a previous session, and can be optimized to modify an individual's performance metric (e.g., to lower or optimize the interference cost under emotional load).

In a non-limiting example, the adaptation of the difficulty of a task and/or interference may be adapted with each different stimulus that is presented as an evocative element.

In another non-limiting example, the example system, method, and apparatus herein can be configured to adapt a difficulty level of a task and/or interference (including the evocative element) one or more times in fixed time intervals or in other set schedule, such as but not limited to each second, in 10 second intervals, every 30 seconds, or on frequencies of once per second, 2 times per second, or more (such as but not limited to 30 times per second).

In an example, the difficulty level of a task or interference can be adapted by changing the time-varying characteristics, such as but not limited to a speed of an object, a rate of change of a facial expression, a direction of trajectory of an object, a change of orientation of an object, at least one color of an object, a type of an object, or a size of an object, or changing a sequence or balance of presentation of a target stimulus versus a non-target stimulus.

In a non-limiting example of a visuo-motor task (a type of navigation task), one or more of navigation speed, shape of the course (changing frequency of turns, changing turning radius), and number or size of obstacles can be changed to modify the difficulty of a navigation game level, with the difficulty level increasing with increasing speed and/or increasing numbers and/or sizes of obstacles (milestone objects).

In a non-limiting example, the difficulty level of a task and/or interference of a subsequent level can also be changed in real-time as feedback, e.g., the difficulty of a subsequent level can be increased or decreased in relation to the data indicative of the performance of the task.

According to the principles herein, the dynamics of tasks and interferences can be presented according to different modes. As a non-limiting examples, the primary task can be presented with an interference at the user interface as a secondary task, requiring a first response from the individual to the primary task in the presence of the interference and a secondary response from the individual to the interference. The first interference is configured to divert the individual's attention from the primary task. The evocative element can be presented in differing modes as a component of either or both the primary task and the interference. In an example, a first mode can be configured such that the primary task includes two or more differing types of evocative elements presented substantially simultaneously at the user interface In an example, a second mode can be configured such that the interference comprises two or more differing types of evocative elements presented substantially simultaneously at the user interface. The individual is instructed not to respond to an evocative element that is configured as a distractor and to respond to an evocative element that is configured as an interruptor. The example system and apparatus is configured to measure data indicative of the physical action of the individual in response to the evocative elements, such that the data comprises at least one measure of emotional processing capabilities of the individual under emotional load. Either or both of the received data indicative of the first response and the secondary response includes the measure of the individual's response to the evocative elements. The example system and apparatus is configured to analyze the data indicative of the first response and the secondary response to compute at least one performance metric comprising at least one quantified indicator of cognitive abilities of the individual under emotional load.

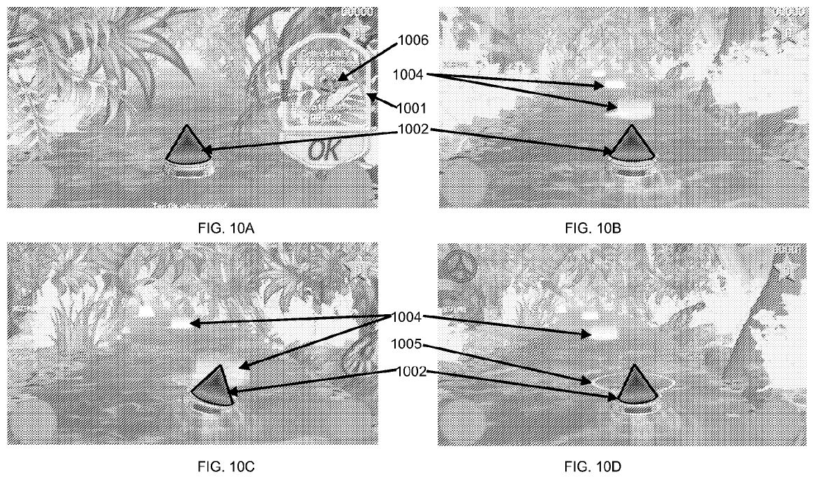

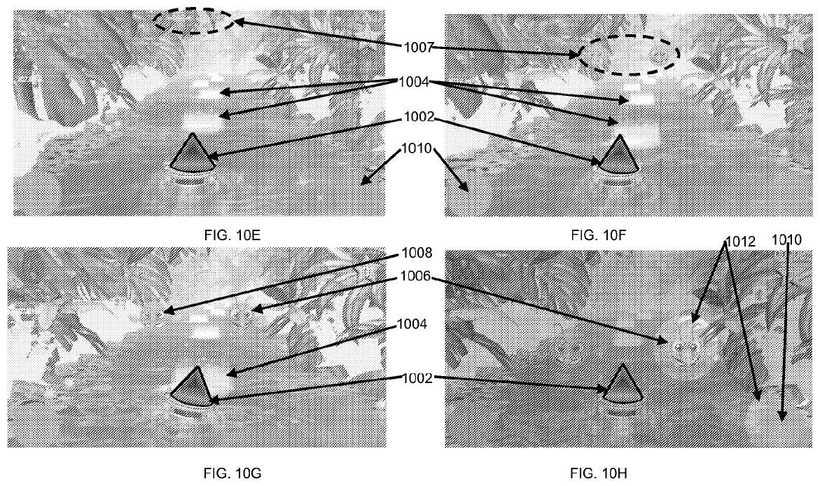

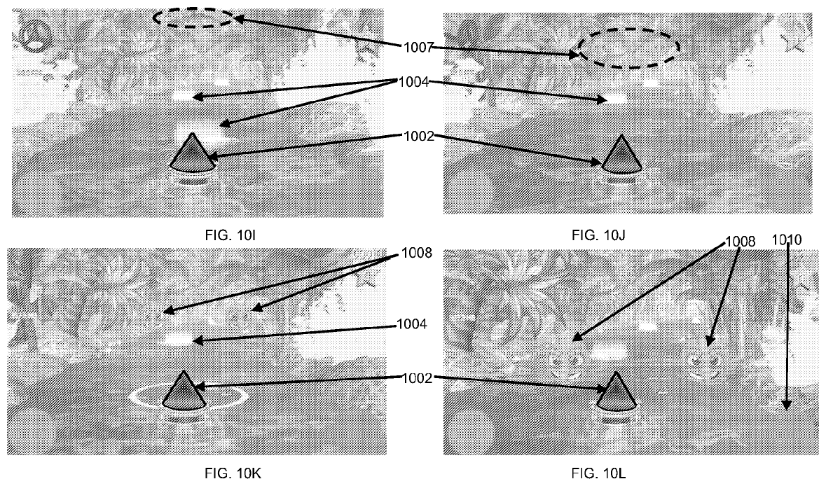

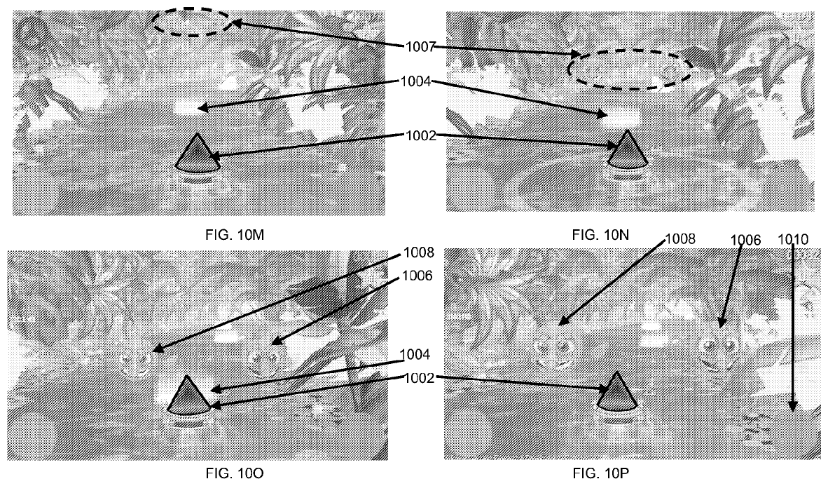

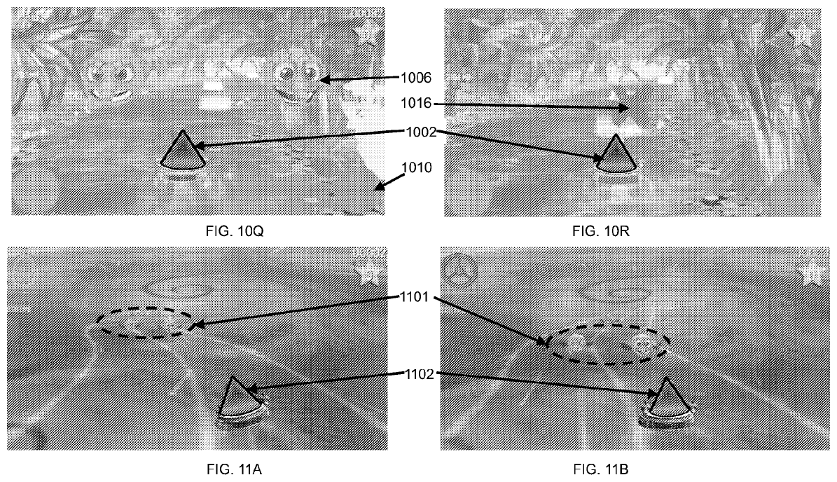

FIGS. 10A-10R show a non-limiting example of the dynamics of tasks and interferences presented according to a first mode, according to the principles herein. In this example, the primary task involves a visuo-motor navigation task, and the interference is target discrimination configured as a secondary task. In this example, the interferences includes evocative elements presented as faces with different facial expressions. In this example, the response to the primary task is measured using sensors (e.g., motion sensor, position sensor, or acceleration sensor) to sense/measure the physical actions of the individual to steer an avatar along a computer-rendered course. The response to the secondary task is measured using sensors (e.g., pressure sensor, contact sensor) to sense/measure the physical actions of the individual to make a selection for target discrimination, such as but not limited to tapping. In the example of FIGS. 10A-10R, the interruptor is configured as a target with specified facial expression, and the distractor is configured as a non-target with any other facial expression. In this non-limiting example dual-face mode shown in FIGS. 10A-10R, two evocative elements are presented substantially simultaneously on portions of the user interface, in differing combinations of target (interruptor) vs non-target (distractor), and requiring the individual to perform physical actions to indicate the target (interruptor) Table 1 describes possible combinations of target/interruptor and non-target/distractor presented on the differing sides (left vs right) of the user interface.

TABLE 1

| Left | Right |
|---|---|
| Target/interruptor | Nontarget/distractor |
| Nontarget/distractor | Target/interruptor |
| Nontarget/distractor | Nontarget/distractor |
| Target/interruptor | Target/interruptor |

As shown in FIG. 10A-10R, specific regions (e.g., the circular portion near the bottom of the user interface) are presented to capture the physical action of the individual to indicate a response to the dual presentation of evocative elements. The user is instructed to tap on the side of the user interface with the target/interruptor evocative element (e.g., to tap on either side if both evocative elements are interruptors), and not to tap on the side of the user interface with the non-target/distractor evocative element (e.g., not to tap if both evocative elements are distractors).

In the example of FIGS. 10A-10R, the system is programmed to display a user interface that includes instructions to the individual to perform the visuo-motor task and target discrimination task. As shown in FIG. 10A, the processing unit is programmed to control a display feature 1001 which includes an identification of the response to the discrimination task. FIGS. 10A-10E show an example of a first time interval of performance of the primary visuo-motor task without interference, where the individual is expected to actuate an apparatus or computing device (or other sensing device) to cause the avatar 1002 to coincide with the milestone object 1004 as the response in the navigation task, with scoring based on the success of the individual at crossing paths with (e.g., hitting) the milestone objects 1004. In another example, the individual is expected to actuate an apparatus or computing device (or other sensing device) to cause the avatar 1002 to miss the milestone object 1004, with scoring based on the success of the individual at avoiding the milestone objects 1004. The processing unit is programmed to show a feature 1005 (see FIG. 10D) that indicates success at interacting with the milestone objects 1004 (the measured response to the visuo-motor task). The interference is a target discrimination task that requires the individual to identify a specific type of facial expression as the interruptor. As shown in FIG. 10A, the processing unit is programmed to control display feature 1001 to display instructions to the individual to identify target object 1006 with a specific facial expression (in this example, a happy face) as the interruptor. The non-target object 1008 is another facial expression (in this example, an angry facial expression) as a distractor. FIGS. 10E-10R show examples of subsequent time intervals of performance of the primary visuo-motor task in the presence of an interference, where the individual is expected to actuate an apparatus or computing device (or other sensing device) to cause the avatar 1002 to coincide with the milestone object 1004 as the response in the navigation task, and also provide a response to the interference, which is configured to include two evocative elements. FIGS. 10E, 10I, and 10M show the initial display of the evocative elements 1007 on a portion of the user interface, where the time-varying characteristic is the trajectory of motion of the objects. In this example, the individual is required to indicate a response to the interference at a specified portion of the user interface (shown as response field 1010) that corresponds to the target object 1006 within the time period for providing the response during the trajectory of the interference. FIGS. 10E-10H show an example where the individual is successful in providing a response to the interference at the response field 1010 corresponding to the target object 1006 (the interruptor), where the processing unit is configured to show a lightened region around both the target object 1006 and the response field 1010 at the user interface as an indication to the individual of the success in providing the response to the interruptor. In FIGS. 10I-10L, the interference is configured as evocative elements that are both non-target objects 1008 (distractors) requiring no response from the individual. FIGS. 10M-10R show an example where the individual is unsuccessful in providing a response at the response field 1010 corresponding to the target object 1006 (the interruptor) within the time period for response. The processing unit is configured to show an “x” at the user interface (see FIG. 10R) as an indication to the individual of the failure to provide the response to the interruptor.

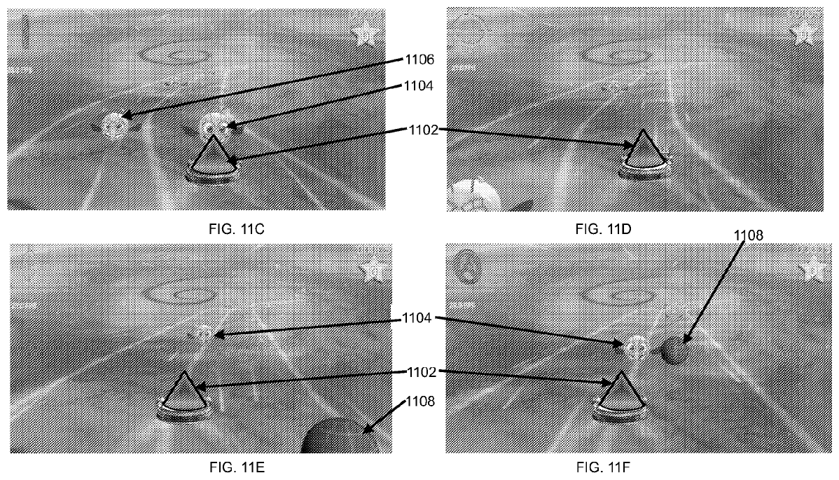

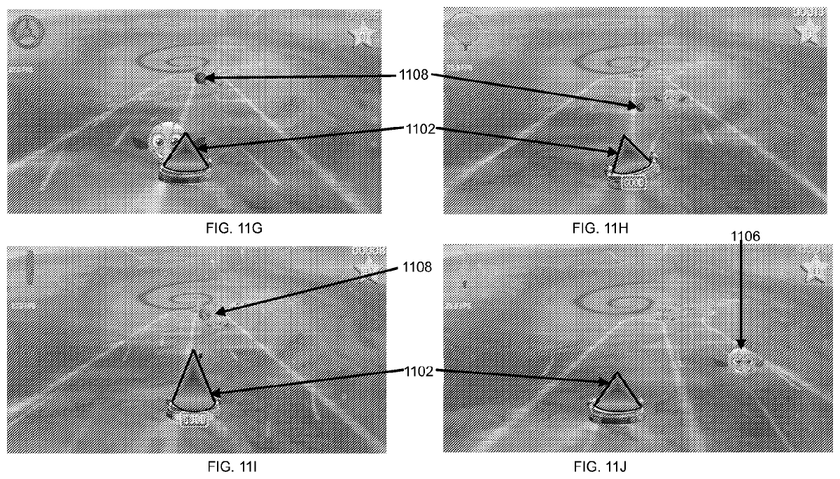

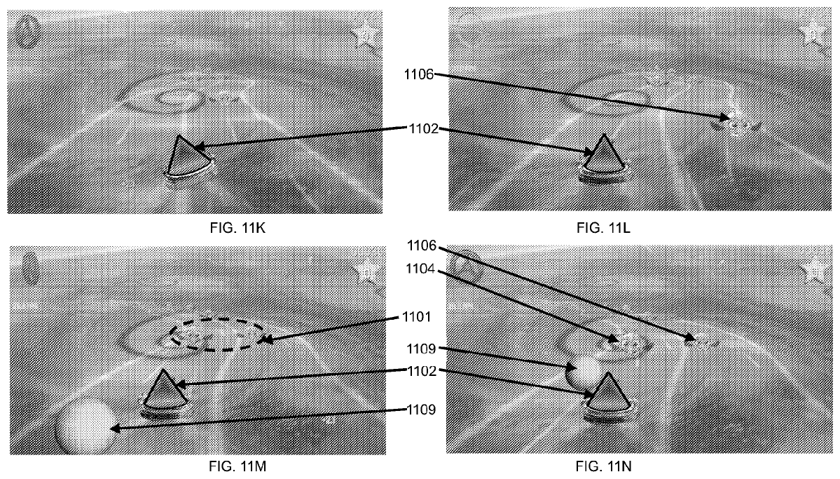

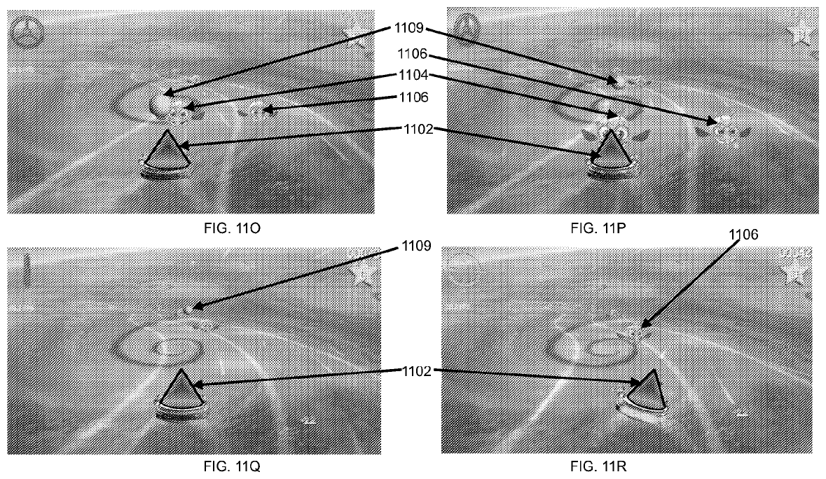

FIGS. 11A-11R show a non-limiting example of the dynamics of tasks and interferences that can be presented according to a second mode at user interfaces, according to the principles herein. In this example, the primary task involves a visuo-motor navigation task, and the interference is target discrimination configured as a secondary task. In an example, the interferences can be non-evocative objects having differing distinct colors, where the individual is instructed to respond based on a target object of a specific color. In other examples, the interferences can be non-evocative objects having differing shapes and/or colors, or other type of object having evocative elements (e.g., having differing facial expressions). In the example of FIGS. 11A-11R, the response to the primary task is measured using sensors (e.g., motion sensor, position sensor, or acceleration sensor) to sense/measure the physical actions of the individual to steer an avatar along a computer-rendered course. The course of the navigation path includes a plurality of evocative elements (faces having differing facial expressions) acting as milestones on the path (e.g., they can be anywhere on a road or other part of landscape/environment). The individual is instructed to interact with an evocative element in the path that has a specified facial expression (one specified type of evocative element as an interruptor) and not to interact with (i.e., avoid) an evocative element in the path that has any other type of facial expression (the other type of evocative elements as distractors). As shown in FIGS. 11A-11R, the evocative elements can be positioned anywhere in the navigation path (e.g., left side, right side, or center). Some of the evocative elements can be presented as two or more evocative elements presented side-by-side, and some of the evocative elements can be presented in single file. Where two or more evocative elements are presented, the evocative elements can be presented as various combinations of interruptors and distractors. The response to the secondary task is measured using sensors (e.g., pressure sensor, contact sensor) to sense/measure the physical actions of the individual to make a selection for target discrimination, such as but not limited to tapping.

In the example of FIGS. 11A-11R, the system is programmed to display a user interface that includes instructions to the individual to perform the visuo-motor task and target discrimination task. In this non-limiting example, the primary visuo-motor task includes the evocative elements, while the target discrimination task does not include evocative elements. FIGS. 11A-11E show an example of a first time interval of performance of the primary visuo-motor task without interference, where the individual is instructed (using a display of the user interface) to actuate an apparatus or computing device (or other sensing device) to cause the avatar 1102 to travel along a path such that it is made to coincide with target milestone objects 1104 and to avoid coinciding with non-target milestone objects 1106. FIG. 11C shows that the target object 1104 is configured as an evocative element with a specific type of facial expression (in this example, a happy facial expression) and the non-target object 1106 is configured as an evocative element with a different type of facial expression (in this example, an angry facial expression). The instructions display of the user interface is used to instruct the individual that the response in the navigation task is dependent on the degree of success at coinciding with target milestone objects 1104, with scoring based on the success of the individual at crossing paths with (e.g., hitting) the target milestone objects 1104. In another example, the processing unit is programmed to control display feature 1101 to display instructions to the individual to actuate an apparatus or computing device (or other sensing device) to cause the avatar 1102 to avoid the non-target milestone object 1104, with scoring based on the success of the individual at avoiding the non-target milestone objects 1106. The interference is a target discrimination task that requires the individual to identify a specific type and/or color of target object as the interruptor. As shown in the non-limiting example of FIG. 11F, the target object 1108 is a geometric object (in this case, a round object) of a specific first non-evocative feature (in this example, the color). As shown in the non-limiting example of FIG. 11MF, the non-target object 1108 is another geometric object of a different (second) non-evocative feature (in this example, the color) as a distractor. In this example, neither the target object nor the non-target object includes an evocative element. FIGS. 11F-11I and 11M-11Q show examples of subsequent time intervals of performance of the primary visuo-motor task in the presence of the interference, where the individual is expected to actuate an apparatus or computing device (or other sensing device) to cause the avatar 1102 to coincide with the target milestone object 1104 as the response in the navigation task, and also to perform a physical action (e.g., tapping) to indicate a response to a target object 1108 as the response to the interference. FIGS. 11F-11I and 11M-11Q also show that the time-varying characteristic of the target objects and non-target objects is the trajectory of motion of the objects. In this example, the individual is required to indicate a response to the interference at the user interface within the time period for providing the response during the trajectory of the interference. FIGS. 11I show an example where the individual is successful in providing a response to the interference corresponding to the target object 1104 (the interruptor), where the processing unit is configured to show a hazy region around the target object 1104 at the user interface as an indication to the individual of the success in providing the response to the interruptor. As shown in FIGS. 11A-11R, the processing unit can be configured to present the target milestone objects 1104 and/or non-target milestone objects 1106 on the left side of the path, on the right side of the path, or at the center. As also shown in FIGS. 11A-11R, the processing unit can be configured to present the target milestone objects 1104 and/or non-target milestone objects on a portion of the path either single-file or side-by-side. FIGS. 11C, 11G, and 11P show examples of successful actuation of an apparatus or computing device (or other sensing device) to cause the avatar 1102 to coincide with the target milestone objects 1104 located at the right-side, center, and left-side, respectively, of the path as the response in performance of the navigation task.

In non-limiting examples herein, the difficulty levels can be adapted by varying certain parameters of the primary tasks and/or the interference, including by varying the difficulty level in discriminating among the evocative element(s). For examples, the target evocative element (e.g., facial expression) can be modulated either dynamically (i.e., in real-time on the user interface) or in differing static renditions to vary the degrees of a facial expression. The dynamic modulation can be achieved through using morphing, which can be effect by various mean. For example, two or more evocative elements on a user interface can be presented with differing degrees of facial expressions, e.g., an extreme (100%) expression of happy or angry or sad, presented with a moderate (50%) expression of happy or angry or sad. The user can be instructed to respond based on a specified degree of the expression as the interruptor and all others as distractors. In another example, the evocative elements can be a blendshapes, with a specified blendshape as the interruptor and all others as distractors. In another example, the evocative element can be presented with a blended combination of facial expressions (non-limiting example is a single face that is shows as part happy and part angry), where the individual is instructed on which degree of facial expression to target as the interruptor and all others are distractors.

In non-limiting examples herein, the difficulty levels can be adapted by varying certain parameters of the primary tasks and/or the interference, including time allowed to make the response, or time it takes the individual to determine a coupling between a facial expression and a non-evocative feature (such as but not limited to the color) of an evocative element. For example, the discrimination of an target/interruptor vs. a non-target/distractor could depend on a coupling between specified non-evocative features (such as but not limited to the colors and/or shapes) for a first session of interaction (where that coupling is not identified to the individual), and this type of coupling is changes in a second session of interaction (e.g., target/interruptor vs. non-target/distractor could be randomized over non-evocative features, such as but not limited to the colors and/or shapes, for the second session of interaction).

According to the principles herein, the dynamics of tasks and interferences can be presented according to different modes based on integration rules. As a non-limiting examples, the primary task can be presented with an interference at the user interface as a secondary task, requiring a first response from the individual to the primary task in the presence of the interference and a secondary response from the individual to the interference. The first interference is configured to divert the individual's attention from the primary task. The user interface is configured to instruct the individual not to respond to an evocative element that is configured as a distractor and to respond to an evocative element that is configured as an interruptor. The interference comprises a plurality of evocative elements presented according to the one or more integration rules, at least one of the evocative elements being configured as a distractor and at least one of the evocative elements being configured as an interruptor. The one or more integration rules are configured such that the plurality of evocative elements are presented with at least two differing non-evocative features (in this example, the colors), each non-evocative feature (such as but not limited to the color) either being correlated with a specific facial expression or not correlated with any facial expressions. The interruptor is configured as an evocative element that has either a specified facial expression or a specified non-evocative feature (such as but not limited to the color). The user interface is configured to measure data indicative of the physical action of the individual in response to the evocative elements, the data comprising at least one measure of emotional processing capabilities of the individual under emotional load. The example system and apparatus is configured to receive data indicative of the first response and the secondary response either or both of the first response and the secondary response comprising the measure of the individual's response to the evocative elements. The example system and apparatus is configured to analyze the data indicative of the first response and the secondary response to compute at least one performance metric comprising at least one quantified indicator of cognitive abilities of the individual under emotional load.

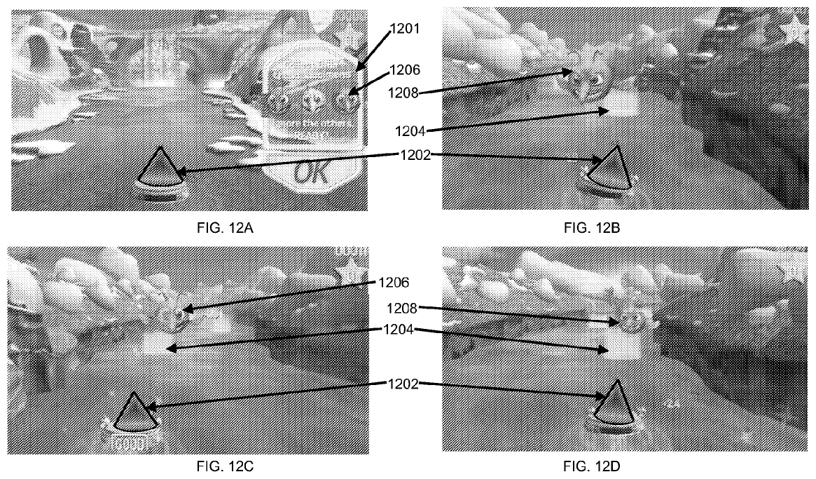

FIGS. 12A-12E show a non-limiting example of the dynamics of tasks and interferences that can be presented according to another mode at user interfaces, according to the principles herein. In this example, the interference is presented as target/interruptor vs. non-target/distractor evocative elements in multiple differing non-evocative features (such as but not limited to the colors). In this example, the example system and apparatus is configured to implement a first integration rule such that there is no correlation between the non-evocative feature (such as but not limited to the color) with the facial expression of the multiple different types of evocative elements. The individual is instructed that the target/interruptor is an evocative element having a specified facial expression, and the non-target/distractor is any other type of evocative element. In the example of FIGS. 12A-12E, the system is programmed to display a user interface that includes instructions to the individual to perform the visuo-motor task and target discrimination task. As shown in FIG. 12A, the processing unit is programmed to control a display feature 1201 which includes an identification of the response to the discrimination task as an evocative element with a happy facial expression, regardless of the non-evocative feature, such as but not limited to the color (i.e., the happy face is the target no matter the color). FIGS. 12A-12E show an example of a time interval of performance of the primary visuo-motor task in the presence of an interference, where the individual is expected to actuate an apparatus or computing device (or other sensing device) to cause the avatar 1202 to coincide with the milestone object 1204 as the response in the navigation task, and also provide a response to the interference. The interference is a target discrimination task that requires the individual to identify an object 1206 with a specific type of facial expression as the interruptor (regardless of the non-evocative feature, such as but not limited to the color, of the object). The non-target object 1208 is another facial expression (in this example, an angry facial expression) as a distractor (also regardless of the non-evocative feature, such as but not limited to the color).

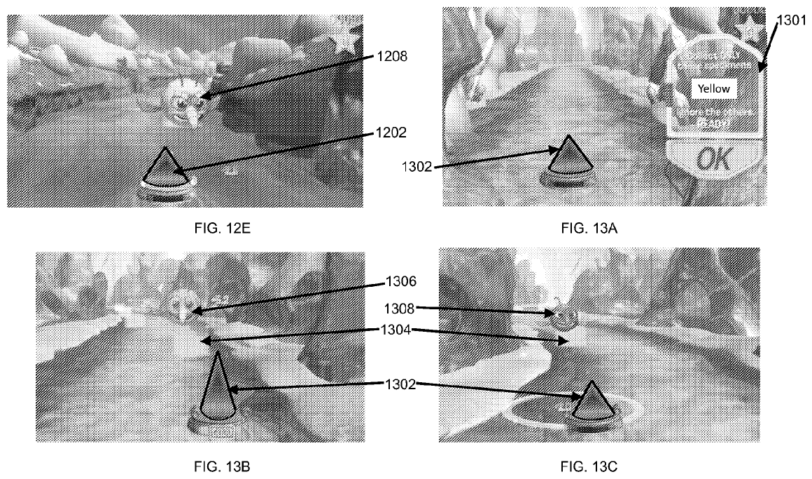

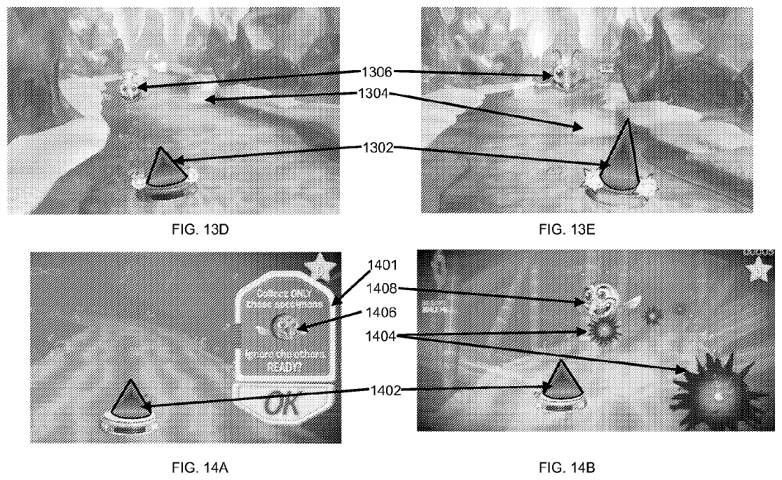

FIGS. 13A-13E show a non-limiting example of the dynamics of tasks and interferences that can be presented according to another mode at user interfaces, according to the principles herein. In this example, the interference is presented as target/interruptor vs. non-target/distractor evocative elements in multiple differing non-evocative features (such as but not limited to the colors). In this example, the example system and apparatus is configured to implement a second integration rule such that there is a correlation between the non-evocative feature (such as but not limited to the color) of the evocative element and the facial expression of the evocative element. The individual is instructed that the target/interruptor is an evocative element having a specified non-evocative feature, such as but not limited to the color (i.e., it is not a based on a facial expression), and the non-target/distractor is an evocative element with any other non-evocative feature, such as but not limited to the color (also not based on facial expression). In the example of FIGS. 13A-13E, the system is programmed to display a user interface that includes instructions to the individual to perform the visuo-motor task and target discrimination task. As shown in FIG. 13A, the processing unit is programmed to control a display feature 1301 which includes an identification of the response to the discrimination task as an evocative element with a specific non-evocative feature, such as but not limited to the color (in this non-limiting example, the color is yellow), regardless of the facial expression of the evocative element. FIGS. 13A-13E show an example of a time interval of performance of the primary visuo-motor task in the presence of an interference, where the individual is expected to actuate an apparatus or computing device (or other sensing device) to cause the avatar 1302 to coincide with the milestone object 1304 as the response in the navigation task, and also provide a response to the interference. The interference is a target discrimination task that requires the individual to identify an object 1306 with a specific non-evocative feature, such as but not limited to the color (yellow) as the interruptor (regardless of the facial expression of the evocative element). The non-target object 1308 is an object 1308 with any other non-evocative feature (such as but not limited to the color) as the distractor.

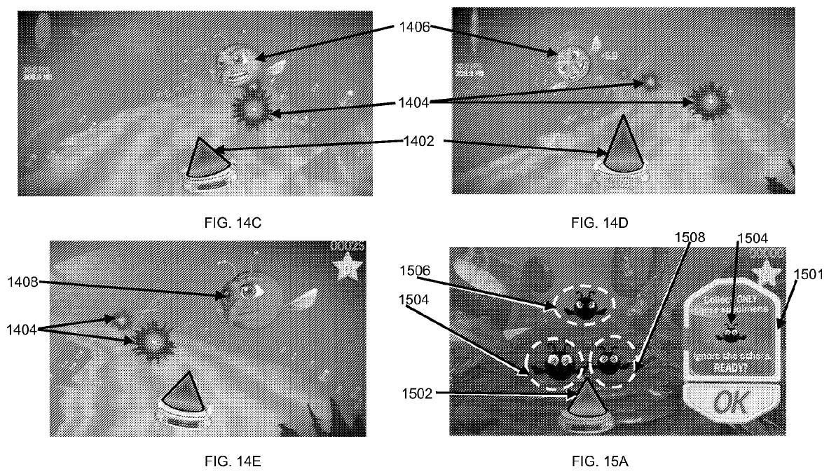

FIGS. 14A-14E show a non-limiting example of the dynamics of tasks and interferences that can be presented according to another mode at user interfaces, according to the principles herein. In this example, the interference is presented as target/interruptor vs. non-target/distractor evocative elements in multiple differing non-evocative features (such as but not limited to the colors). In this example, the example system and apparatus is configured to implement a third integration rule such that there is a correlation between the non-evocative feature (such as but not limited to the color) of the evocative element and the facial expression of the evocative element. The individual is instructed that the target/interruptor is an evocative element having a specified facial expression (i.e., it is not a based on a non-evocative feature, such as but not limited to the color), and the non-target/distractor is an evocative element with any other facial expression (also not based on a non-evocative feature, such as but not limited to the color). In the example of FIGS. 14A-14E, the system is programmed to display a user interface that includes instructions to the individual to perform the visuo-motor task and target discrimination task. As shown in FIG. 14A, the processing unit is programmed to control a display feature 1401 which includes an identification of the response to the discrimination task as an evocative element of a specified non-evocative feature, such as but not limited to the color (in this example, green) and having an angry facial expression (i.e., the facial expression of the target Is correlated with the color). FIGS. 14A-14E show an example of a time interval of performance of the primary visuo-motor task in the presence of an interference, where the individual is expected to actuate an apparatus or computing device (or other sensing device) to cause the avatar 1402 to coincide with the milestone object 1404 as the response in the navigation task, and also provide a response to the interference. The interference is a target discrimination task that requires the individual to identify an object 1406 with a specific type of facial expression (angry) and specific non-evocative feature, such as but not limited to the color (green) as the interruptor. The non-target object 1408 is another facial expression and color as the distractor (in this example, a yellow face with happy facial expression or a red face with a neutral facial expression).

FIGS. 15A-15E show a non-limiting example of the dynamics of tasks and interferences that can be presented according to another mode at user interfaces, according to the principles herein. In this example, the interference is presented as target/interruptor vs. non-target/distractor evocative elements in a single non-evocative feature (such as but not limited to the color), but with facial expression being varied using primarily the eyes of the evocative element, where the eyes make the expressions (e.g., not by a shape of a mouth), and remainder of rendered creature/face is a blank, dark, or neutral color. In another example, the evocative element may be configured such that the mouth makes the expression, or both the eyes and mouth are configured to make the expression. In this example, the color is black, however, it can be any other color such as but not limited to brown, blue, white, or other neutral color. The individual is instructed that the target/interruptor is an evocative element having a specified facial expression made by the eyes, and the non-target/distractor is an evocative element with any other facial expression expressed by the eyes. In the example of FIGS. 15A-15E, the system is programmed to display a user interface that includes instructions to the individual to perform the visuo-motor task and target discrimination task. As shown in FIG. 15A, the processing unit is programmed to control a display feature 1501 which includes an identification of the response to the discrimination task as target object 1504 with a happy facial expression (as expressed by the eyes) as the interruptor. The non-target target objects 1506 and 1508 with the angry and neutral facial expressions, respectively, as the distractors. FIGS. 15A-15E show an example of a time interval of performance of the primary visuo-motor task in the presence of an interference, where the individual is expected to actuate an apparatus or computing device (or other sensing device) to cause the avatar 1502 to coincide with the milestone object 1510 as the response in the navigation task, and also provide a response to the interference. The interference is a target discrimination task that requires the individual to identify the target object 1506.

In another non-limiting example, the interference can be presented as target/interruptor vs. non-target/distractor evocative elements in multiple differing non-evocative features (such as but not limited to the colors or the shape) and having differing facial expressions (such as but not limited to the angry, happy, neutral). In this example, the example system and apparatus is configured to implement a fourth integration rule such that there is no correlation between the non-evocative feature (such as but not limited to the color or shape) of the evocative element and the facial expression of the evocative element. The individual is instructed that the target/interruptor is an evocative element that has either (i) a specified facial expression (i.e., it is not a based on a non-evocative feature, such as but not limited to the color), or (ii) a specified non-evocative feature, such as but not limited to the color and/or a shape (i.e., it is not a based on a facial expression), but not both the specified facial expression and the specified non-evocative feature (which the individual is instructed to be treated as a non-target/distractor). For example, the target/interruptor is an evocative element that has either a square shape or a happy facial expression, but not both a happy facial expression and a square shape (which the individual is instructed to be treated as a non-target/distractor). The individual is instructed that the non-target/distractor is an evocative element with any other facial expression and any other non-evocative feature.

In another example, the individual may be instructed that the target/interruptor is an evocative element that has a specified first facial expression in a first specified non-evocative feature (including shape and/or color) and a specified second facial expression in a second specified non-evocative feature (including shape and/or color), and the non-target/distractor is an evocative element of any other facial expression or non-evocative feature.

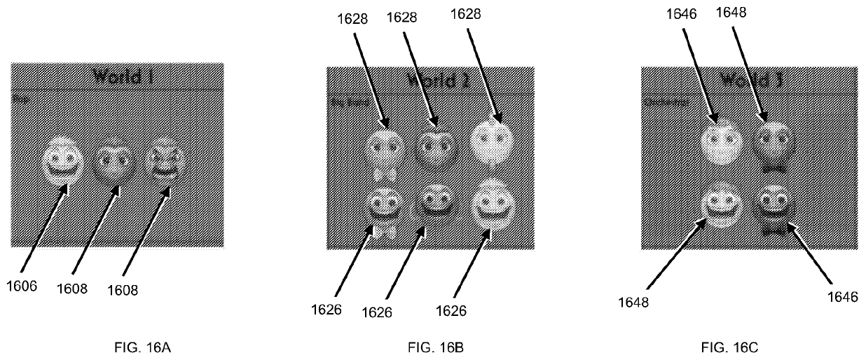

FIGS. 16A-16C show an example implementation that includes multiple differing sessions configured according to differing implementation rules. In the non-limiting example of FIG. 16A, the system and apparatus is configured to implement the second integration rule such that there is a correlation between the non-evocative feature (such as but not limited to the color) of the evocative element and the facial expression of the evocative element. The primary and/or secondary task requires the individual to identify an object 1606 with a specific non-evocative feature, such as but not limited to the color (yellow) as the interruptor (regardless of the facial expression of the evocative element). The non-target object 1608 is an object 1608 with any other non-evocative feature (such as but not limited to the color) as the distractor. The description and features described in connection with FIGS. 13A-13E apply to FIG. 16A. In the non-limiting example of FIG. 16B, the system and apparatus is configured to implement the first integration rule such that there is no correlation between the non-evocative feature (such as but not limited to the color) with the facial expression of the multiple different types of evocative elements. The individual is instructed that the target/interruptor is an evocative element having a specified facial expression, and the non-target/distractor is any other type of evocative element. The primary and/or secondary task requires the individual to identify an object 1626 with a specific type of facial expression as the interruptor (regardless of the non-evocative feature, such as but not limited to the color, of the object). The non-target object 1628 is another facial expression (in this example, a neutral facial expression) as a distractor (also regardless of the non-evocative feature, such as but not limited to the color). The description and features described in connection with FIGS. 12A-12E apply to FIG. 16B. In the non-limiting example of FIG. 16C, the system and apparatus is configured to implement the fourth integration rule such that the evocative element has a specified color and/or a specified facial expression as the target, while the non-target is: (i) an evocative element having any color other than the specified color, (ii) an evocative element having any facial expression other than the specified facial expression, and (iii) an evocative element having both the specified color and the specified facial expression. The primary and/or secondary task requires the individual to identify an object 1646 with a specific type of facial expression or a specific color as the interruptor. The non-target object 1648 is (i) an evocative element having any color other than the specified color, (ii) an evocative element having any facial expression other than the specified facial expression, and (iii) an evocative element having both the specified color and the specified facial expression. The description and features described in connection with the fourth integration rule hereinabove apply to FIG. 16C.

In non-limiting examples, the mode or integration rules can be varied from one session of interaction to another, and/or from one trial to another. For example, the adapting of difficulty levels to the performance of the individual can be effected by changing from one integration rule and/or target mode to another between two or more different trials or sessions. In another example, the adapting of difficulty levels to the performance of the individual can be effected by changing from one integration rule and/or target mode to another within a given trial or a given session.

In an example, the interference cost can include a computation of an emotional interference cost measure, to provide a measure of an individual's capability to recognize the emotional state of the facial expression of an evocative element while performing another task vs while not performing the other task. For example, the emotional interference cost measure can be computed based on data collected as a measure of the individual's physical actions to recognize/discriminate an evocative element while perform a navigation task (e.g., while steering) vs data collected as a measure of the individual's physical actions to recognize/discriminate an evocative element while not performing the navigation task (e.g., while not steering). In an example, the value of the emotional interference cost measure can be used as biomarker of an individual's cognitive condition, or disorder.

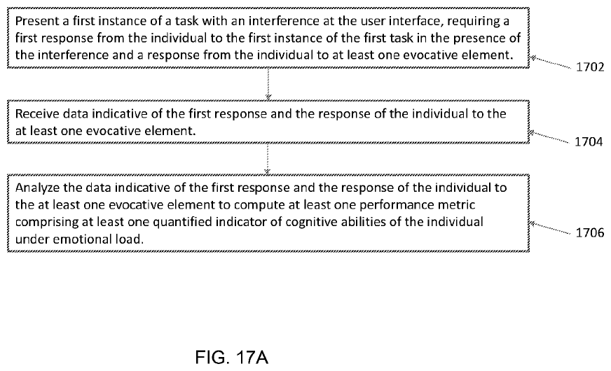

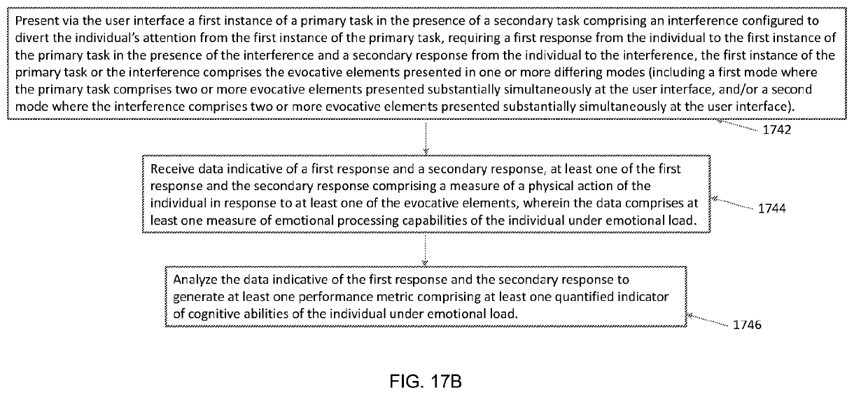

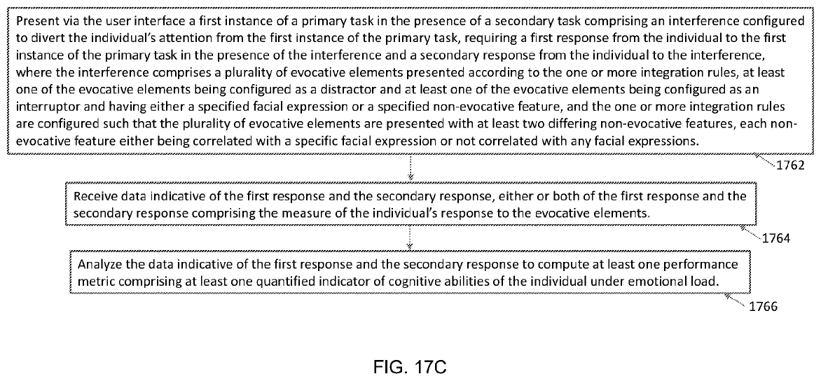

FIGS. 17A-17C show flowcharts of non-limiting example methods using a cognitive platform configured for using evocative elements rendered in modes, according to the principles herein.

FIG. 17A shows a flowchart of a non-limiting example method that can be implemented using a platform product that includes at least one processing unit. In block 1702, the at least one processing unit Is used to present at least one user interface to render a first instance of a task with a first interference at the user interface, requiring a first response from the individual to the first instance of the first task in the presence of the first interference and a response from the individual to at least one evocative element. For example, the at least one processing unit Is used to render at least one graphical user interface to present a computerized stimuli or interaction (CSI) or other interactive elements to the user, or cause an actuating component of the platform product to effect auditory, tactile, or vibrational computerized elements (including CSIs) to effect the stimulus or other interaction with a user. The first instance of the first task and/or the first interference can include the at least one evocative element. The user interface is configured to measure data indicative of the response of the individual to the at least one evocative element (where the data includes at least one measure of emotional processing capabilities of the individual under emotional load). The apparatus is configured to measure substantially simultaneously a first response from the individual to the first instance of the first task and the response from the individual to the at least one evocative element. In block 1704, the at least one processing unit is used to cause a component of the program product to receive data indicative of the first response and the response of the individual to the at least one evocative element. For example, the at least one processing unit is used to cause a component of the program product to receive data indicative of at least one user response based on the user interaction with the CSI or other interactive element (such as but not limited to cData) In an example where at least one graphical user interface is rendered to present the computerized stimuli or interaction (CSI) or other interactive elements to the user, the at least one processing unit can be programmed to cause graphical user interface to receive the data indicative of at least one user response. In block 1706, the at least one processing unit is used to cause a component of the program product to analyze the data indicative of the first response and the response of the individual to the at least one evocative element to compute at least one performance metric comprising at least one quantified indicator of cognitive abilities of the individual under emotional load. For example, the at least one processing unit also can be used to: analyze the differences in the individual's performance based on determining the differences between the user's responses, and/or adjust the difficulty level of the computerized stimuli or interaction (CSI) or other interactive elements based on the individual's performance determined in the analysis, and/or provide an output or other feedback from the platform product indicative of the individual's performance, and/or cognitive assessment, and/or response to cognitive treatment. In some examples, the results of the analysis may be used to modify the difficulty level or other property of the computerized stimuli or interaction (CSI) or other interactive elements.

FIG. 17B shows a flowchart of another non-limiting example method that can be implemented using a platform product that includes at least one processing unit. In block 1742, the at least one processing unit Is used to present via the user interface a first instance of a primary task in the presence of a secondary task comprising an interference configured to divert the individual's attention from the first instance of the primary task, requiring a first response from the individual to the first instance of the primary task in the presence of the interference and a secondary response from the individual to the interference, the first instance of the primary task or the interference comprises the evocative elements presented in one or more differing modes (including a first mode where the primary task comprises two or more evocative elements presented substantially simultaneously at the user interface, and/or a second mode where the interference comprises two or more evocative elements presented substantially simultaneously at the user interface). In block 1744, the at least one processing unit Is used to receive data indicative of a first response and a secondary response, at least one of the first response and the secondary response comprising a measure of a physical action of the individual in response to at least one of the evocative elements, wherein the data comprises at least one measure of emotional processing capabilities of the individual under emotional load. In block 1746, the at least one processing unit Is used to analyze the data indicative of the first response and the secondary response to generate at least one performance metric comprising at least one quantified indicator of cognitive abilities of the individual under emotional load.

FIG. 17C shows a flowchart of another non-limiting example method that can be implemented using a platform product that includes at least one processing unit. In block 1762, the at least one processing unit Is used to present via the user interface a first instance of a primary task in the presence of a secondary task comprising an interference configured to divert the individual's attention from the first instance of the primary task, requiring a first response from the individual to the first instance of the primary task in the presence of the interference and a secondary response from the individual to the interference, where the interference comprises a plurality of evocative elements presented according to the one or more integration rules, at least one of the evocative elements being configured as a distractor and at least one of the evocative elements being configured as an interruptor and having either a specified facial expression or a specified non-evocative feature, and the one or more integration rules are configured such that the plurality of evocative elements are presented with at least two differing non-evocative features, each non-evocative feature either being correlated with a specific facial expression or not correlated with any facial expressions. In block 1764, the at least one processing unit Is used to receive data indicative of the first response and the secondary response, either or both of the first response and the secondary response comprising the measure of the individual's response to the evocative elements. In block 1766, the at least one processing unit Is used to analyze the data indicative of the first response and the secondary response to compute at least one performance metric comprising at least one quantified indicator of cognitive abilities of the individual under emotional load.

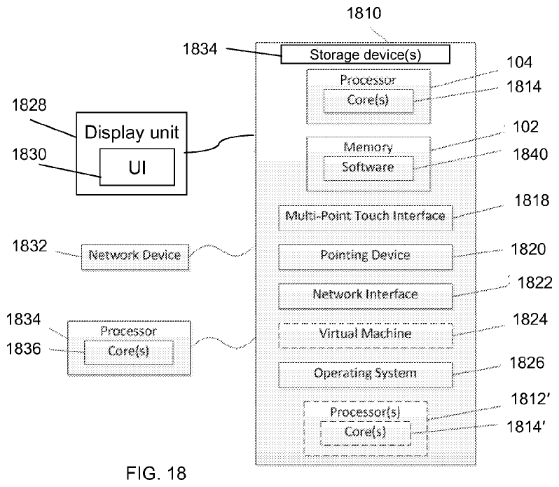

FIG. 18 is a block diagram of an example computing device 1810 that can be used as a computing component according to the principles herein. In any example herein, computing device 1810 can be configured as a console that receives user input to implement the computing component, including to apply the signal detection metrics in computer-implemented adaptive response-deadline procedures. For clarity, FIG. 18 also refers back to and provides greater detail regarding various elements of the example system of FIG. 1 and the example computing device of FIG. 2. The computing device 1810 can include one or more non-transitory computer-readable media for storing one or more computer-executable instructions or software for implementing examples. The non-transitory computer-readable media can include, but are not limited to, one or more types of hardware memory, non-transitory tangible media (for example, one or more magnetic storage disks, one or more optical disks, one or more flash drives), and the like. For example, memory 102 included in the computing device 1810 can store computer-readable and computer-executable instructions or software for performing the operations disclosed herein. For example, the memory 102 can store a software application 1840 which is configured to perform various of the disclosed operations (e.g., analyze cognitive platform measurement data and response data (including response to the evocative element), compute a performance metric (including an interference cost) under emotional load, or perform other computation as described herein). In an example, the interference cost can include a computation of the emotional interference cost measure. The computing device 1810 also includes configurable and/or programmable processor 104 and an associated core 1814, and optionally, one or more additional configurable and/or programmable processing devices, e.g., processor(s) 1812′ and associated core(s) 1814′ (for example, in the case of computational devices having multiple processors/cores), for executing computer-readable and computer-executable instructions or software stored in the memory 102 and other programs for controlling system hardware. Processor 104 and processor(s) 1812′ can each be a single core processor or multiple core (1814 and 1814′) processor.

Virtualization can be employed in the computing device 1810 so that infrastructure and resources in the console can be shared dynamically. A virtual machine 1824 can be provided to handle a process running on multiple processors so that the process appears to be using only one computing resource rather than multiple computing resources. Multiple virtual machines can also be used with one processor.

Memory 102 can include a computational device memory or random access memory, such as DRAM, SRAM, EDO RAM, and the like. Memory 102 can include other types of memory as well, or combinations thereof.

A user can interact with the computing device 1810 through a visual display unit 1828, such as a computer monitor, which can display one or more user interfaces (UI) 1830 that can be provided in accordance with example systems and methods. The computing device 1810 can include other I/O devices for receiving input from a user, for example, a keyboard or any suitable multi-point touch interface 1818, a pointing device 1820 (e.g., a mouse). The keyboard 1818 and the pointing device 1820 can be coupled to the visual display unit 1828 The computing device 1810 can include other suitable conventional I/O peripherals.

The computing device 1810 can also include one or more storage devices 1834, such as a hard-drive, CD-ROM, or other computer readable media, for storing data and computer-readable instructions and/or software that perform operations disclosed herein. Example storage device 1834 can also store one or more databases for storing any suitable information required to implement example systems and methods. The databases can be updated manually or automatically at any suitable time to add, delete, and/or update one or more items in the databases.

The computing device 1810 can include a network interface 1822 configured to interface via one or more network devices 1832 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1822 can include a built-in network adapter, network interface card, PCMCIA network card, card bus network adapter, wireless network adapter, USB network adapter, modem or any other device suitable for interfacing the computing device 1810 to any type of network capable of communication and performing the operations described herein. Moreover, the computing device 1810 can be any computational device, such as a workstation, desktop computer, server, laptop, handheld computer, tablet computer, or other form of computing or telecommunications device that is capable of communication and that has sufficient processor power and memory capacity to perform the operations described herein.

The computing device 1810 can run any operating system 1826, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, or any other operating system capable of running on the console and performing the operations described herein. In some examples, the operating system 1826 can be run in native mode or emulated mode. In an example, the operating system 1826 can be run on one or more cloud machine instances.

Examples of the systems, methods and operations described herein can be implemented in digital electronic circuitry, or in computer software, firmware, or hardware, including the structures disclosed in this specification and their structural equivalents, or in combinations of one or more thereof. Examples of the systems, methods and operations described herein can be implemented as one or more computer programs, i.e., one or more modules of computer program instructions, encoded on computer storage medium for execution by, or to control the operation of, data processing apparatus. The program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” or “computing device” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, application or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing on one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), for example. Devices suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto-optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, for displaying information to the user and a keyboard and a pointing device, e.g., a mouse, a stylus, touch screen or a trackball, by which the user can provide input to the computer. Other kinds of devices can be used to provide for interaction with a user as well. For example, feedback (i.e., output) provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

In some examples, a system, method or operation herein can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Example computing system 400 can include clients and servers. A client and server are generally remote from each other and typically interact through a communication network. The relationship of client and server arises by virtue of computer programs running on the respective computers and having a client-server relationship to each other. In some embodiments, a server transmits data to a client device (e.g., for purposes of displaying data to and receiving user input from a user interacting with the client device). Data generated at the client device (e.g. a result of the user interaction) can be received from the client device at the server.

### CONCLUSION

The above-described embodiments can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single computer or distributed among multiple computers.

In this respect, various aspects of the invention may be embodied at least in part as a computer readable storage medium (or multiple computer readable storage media) (e.g, a computer memory, compact disks, optical disks, magnetic tapes, flash memories, circuit configurations in Field Programmable Gate Arrays or other semiconductor devices, or other tangible computer storage medium or non-transitory medium) encoded with one or more programs that, when executed on one or more computers or other processors, perform methods that implement the various embodiments of the technology discussed above. The computer readable medium or media can be transportable, such that the program or programs stored thereon can be loaded onto one or more different computers or other processors to implement various aspects of the present technology as discussed above.

The terms “program” or “software” are used herein in a generic sense to refer to any type of computer code or set of computer-executable instructions that can be employed to program a computer or other processor to implement various aspects of the present technology as discussed above. Additionally, it should be appreciated that according to one aspect of this embodiment, one or more computer programs that when executed perform methods of the present technology need not reside on a single computer or processor, but may be distributed in a modular fashion amongst a number of different computers or processors to implement various aspects of the present technology.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system for generating an indication of cognitive skills in an individual using evocative elements presented in one or more differing modes, the system comprising:
one or more processors; and
a memory to store processor-executable instructions and communicatively coupled with the one or more processors,
wherein upon execution of the processor-executable instructions by the one or more processors, the one or more processors are configured to:
generate a user interface;
present via the user interface a first instance of a primary task in the presence of a secondary task comprising an interference configured to divert the individual's attention from the first instance of the primary task, requiring a first response from the individual to the first instance of the primary task in the presence of the interference and a secondary response from the individual to the interference;
wherein:
the first instance of the primary task or the interference comprises the evocative elements presented in the one or more differing modes comprising at least one of:
a first mode wherein the primary task comprises two or more evocative elements presented substantially simultaneously at the user interface; or
a second mode wherein the interference comprises two or more evocative elements presented substantially simultaneously at the user interface;
receive data indicative of a first response and a secondary response, at least one of the first response and the secondary response comprising a measure of a physical action of the individual in response to at least one of the evocative elements, wherein the data comprises at least one measure of emotional processing capabilities of the individual under emotional load; and
analyze the data indicative of the first response and the secondary response to generate at least one performance metric comprising at least one quantified indicator of cognitive abilities of the individual under emotional load.

2. The system of claim 1, wherein the one or more processors are configured to configure the user interface to instruct the individual not to respond to an evocative element that is configured as a distractor.

3. The system of claim 1, wherein the one or more processors are configured to configure the user interface to instruct the individual to respond to an evocative element that is configured as an interruptor.

4. A system for generating an indication of cognitive skills in an individual using evocative elements presented according to one or more integration rules, the system comprising:
one or more processors; and
a memory to store processor-executable instructions and communicatively coupled with the one or more processors,
wherein upon execution of the processor-executable instructions by the one or more processors, the one or more processors are configured to:
generate a user interface;
present via the user interface a first instance of a primary task in the presence of a secondary task comprising an interference configured to divert the individual's attention from the first instance of the primary task, requiring a first response from the individual to the first instance of the primary task in the presence of the interference and a secondary response from the individual to the interference;
wherein:
the interference comprises a plurality of evocative elements presented according to the one or more integration rules, at least one of the evocative elements being configured as a distractor and at least one of the evocative elements being configured as an interruptor and having either a specified facial expression or a specified non-evocative feature; and
the one or more integration rules are configured such that the plurality of evocative elements are presented with at least two differing non-evocative features, each non-evocative feature either being correlated with a specific facial expression or not correlated with any facial expressions;
receive data indicative of the first response and the secondary response, at least one of the first response and the secondary response comprising a measure of the individual's response to the evocative elements, wherein (i) the response comprises a physical action of the individual in response to the evocative elements and (ii) the data comprises at least one measure of emotional processing capabilities of the individual under emotional load; and
analyze the data indicative of the first response and the secondary response to generate at least one performance metric comprising at least one quantified indicator of cognitive abilities of the individual under emotional load.

5. The system of claim 4, wherein the one or more processors are configured to configure the user interface to instruct the individual not to respond to an evocative element that is configured as a distractor.

6. The system of claim 4, wherein the one or more processors are configured to configure the user interface to instruct the individual to respond to an evocative element that is configured as an interruptor.

7. The system of claim 4, wherein the non-evocative feature is at least one of color or a shape.

8. The system of claim 4, wherein the one or more integration rules comprise a first integration rule that requires each evocative element to be presented with a non-evocative feature that is not correlated with a facial expression, the interruptor comprising an evocative element having a specified facial expression.

9. The system of claim 4, wherein the one or more integration rules comprise a second integration rule that requires each evocative element to be presented with a non-evocative feature that is correlated with a facial expression, the interruptor comprising an evocative element having a specified non-evocative feature.

10. The system of claim 4, wherein the one or more integration rules comprise a third integration rule that requires each evocative element to be presented with a non-evocative feature that is correlated with a facial expression, the interruptor comprising an evocative element having a specified facial expression.

11. The system of claim 4, wherein the one or more integration rules comprise a fourth integration rule that requires each evocative element to be presented with a single non-evocative feature with differing facial expressions based on eyes and/or mouth, the interruptor comprising an evocative element having a specified facial expression.

12. The system of claim 1 or 4, wherein the one or more processors are further configured to:
present via the user interface a second instance of the primary task without the interference, requiring a second response from the individual to the second instance of the primary task; and
analyze the data indicative of the first response, the second response, and the secondary response to generate the at least one performance metric.

13. The system of claim 1 or 4, wherein the primary task is a continuous visuo-motor task.

14. The system of claim 1 or 4, wherein the interference is a target discrimination task.

15. The system of claim 1 or 4, wherein at least one of the first response and the secondary response comprises the measured data indicative of the physical action of the individual.

16. The system of claim 1 or 4, wherein the one or more processors are further configured to at least one of: (i) generate an output representing the at least one generated performance metric or (ii) transmit to a computing device the at least one generated performance metric.

17. The system of claim 1 or 4, wherein the one or more processors are further configured to:
present via the user interface a second instance of the primary task, requiring a second response from the individual to the second instance of the primary task; and
analyze a difference between the data indicative of the first response and the second response to compute an interference cost as a measure of at least one additional indication of cognitive abilities of the individual.

18. The system of claim 17, wherein in the first instance the primary task is continuous and rendered over a first time interval, in the second instance the primary task is continuous and rendered over a second time interval, and the first time interval is different from the second time interval.

19. The system of claim 18, wherein the measure of cognitive capabilities of the individual is computed based on at least one of a measure of the individual's capability to distinguish among differing types of evocative elements, or a measure of the individual's capability to distinguish among evocative elements having differing valence.

20. The system of claim 1 or 4, wherein the one or more processors configure at least one evocative element as a temporally overlapping task with at least one of the first instance of the primary task or the interference.

21. The system of claim 1 or 4, wherein the one or more processors are further configured to generate a predictive model based on values of the at least one generated performance metric, to generate a predictive model output indicative of a measure of cognition, a mood, a level of cognitive bias, or an affective bias of the individual.

22. The system of claim 21, wherein the predictive model comprises at least one of a linear/logistic regression, principal component analysis, generalized linear mixed models, random decision forests, support vector machines, or artificial neural networks.

23. The system of claim 1 or 4, wherein at least one evocative element comprises an image of a face that represents or correlates with an expression of a specific emotion or a combination of emotion

24. The system of claim 1 or 4, wherein the primary task or the interference comprises an adaptive response-deadline procedure having a response-deadline; and the one or more processors are further configured to modify the response-deadline of the at least one adaptive response-deadline procedure to adjust a difficulty level of the primary task or the interference.

25. The system of claim 24, wherein the one or more processors are configured to control the user interface to modify a temporal length of the response window associated with the response-deadline procedure.

26. The system of claim 24, wherein adjusting the difficulty level comprises applying an adaptive algorithm to progressively adjust a level of valence of the at least one evocative element

27. The system of claim 1 or 4, wherein the generated performance metric comprises an indicator of a projected response of the individual to a cognitive treatment.

28. The system of claim 1 or 4, wherein the generated performance metric comprises a quantitative indicator of at least one of a mood, a cognitive bias, or an affective bias of the individual.

29. The system of claim 1 or 4, wherein the one or more processors are further configured to use the at least one first performance metric to at least one of (i) recommend a change of at least one of an amount, concentration, or dose titration of a pharmaceutical agent, drug, or biologic, (ii) identify a likelihood of the individual experiencing an adverse event in response to administration of the pharmaceutical agent, drug, or biologic, (iii) identify a change in the individual's cognitive response capabilities, (iv) recommend a treatment regimen, or (v) recommend or determine a degree of effectiveness of at least one of a behavioral therapy, counseling, or physical exercise.

30. The system of claim 1 or 4, wherein the data is indicative of the first response and the secondary response at a first difficulty level, and the one or more processors are further configured to:
analyze data indicative of the first response and the secondary response at a second difficulty level to generate at least one second performance metric representative of a performance of the individual of interference processing under emotional load.

31. The system of claim 1 or 4, wherein the one or more processors are further configured to:
measure substantially simultaneously the first response from the individual to the first instance of the task, a secondary response of the individual to the interference, and the response to the at least one evocative element; and
generate the performance metric based on the first response, secondary response, and the response to the at least one evocative element.

32. The system of claim 1 or 4, wherein the one or more processors are further configured to:
adjust a difficulty of at least one of the task or the interference based on the at least one generated first performance metric such that the primary task with the interference are rendered at a second difficulty level; and
generate a second performance metric representative of cognitive abilities of the individual under emotional load based at least in part on the data indicative of the first response and the response of the individual to the at least one evocative element.

33. The system of any of claims 1-32, wherein the system is at least one of a virtual reality system, an augmented reality system, or a mixed reality system.

34. The system of any one of claims 1-32, further comprising:
one or more physiological components,
wherein upon execution of the processor-executable instructions by the one or more processors, the one or more processors:
receive data indicative of one or more measurements of the physiological component; and
analyze the data indicative of the first response and the response of the individual to the at least one evocative element, and the data indicative of one or more measurements of the physiological component to generate the first performance metric.

35. The system of claim 34, wherein the one or more physiological components is configured to measure data indicative of one or more of heart rate, skin conductance, reaction time, and accelerometer measurements.

36. A system for enhancing cognitive skills in an individual using evocative elements presented in one or more differing modes, the system comprising:
one or more processors; and
a memory to store processor-executable instructions and communicatively coupled with the one or more processors,
wherein upon execution of the processor-executable instructions by the one or more processors, the one or more processors are configured to:
generate a user interface;
present via the user interface, at a first difficulty level in a first iteration, a first instance of a primary task in the presence of a secondary task comprising an interference configured to divert the individual's attention from the first instance of the primary task, requiring a first response from the individual to the first instance of the primary task in the presence of the interference and a secondary response from the individual to the interference;
wherein:
the first instance of the primary task or the interference comprises the evocative elements presented in the one or more differing modes comprising at least one of:
a first mode wherein the primary task comprises two or more evocative elements presented substantially simultaneously at the user interface; or
a second mode wherein the interference comprises two or more evocative elements presented substantially simultaneously at the user interface;
receive data indicative of a first response and a secondary response, at least one of the first response and the secondary response comprising a measure of a physical action of the individual in response to at least one of the evocative elements, wherein the data comprises at least one measure of emotional processing capabilities of the individual under emotional load;
analyze the data indicative of the first response and the secondary response to generate a first performance metric comprising at least one quantified indicator of cognitive abilities of the individual under emotional load;
adjust a difficulty of at least one of the primary task or the interference based on the generated at least one first performance metric such that the primary task with the interference are rendered at a second difficulty level in a second iteration; and
generate a second performance metric representative of cognitive abilities of the individual under emotional load based at least in part on the data indicative of the first response and the second response from the second iteration to provide an indication of a difference in cognitive abilities of the individual.

37. A system for enhancing cognitive skills in an individual using evocative elements presented according to one or more integration rules, the system comprising:
one or more processors; and
a memory to store processor-executable instructions and communicatively coupled with the one or more processors,
wherein upon execution of the processor-executable instructions by the one or more processors, the one or more processors are configured to:
generate a user interface;
present via the user interface, at a first difficulty level in a first iteration, a first instance of a primary task in the presence of a secondary task comprising an interference configured to divert the individual's attention from the first instance of the primary task, requiring a first response from the individual to the first instance of the primary task in the presence of the interference and a secondary response from the individual to the interference;
wherein:
the interference comprises a plurality of evocative elements presented according to the one or more integration rules, at least one of the evocative elements being configured as a distractor and at least one of the evocative elements being configured as an interruptor and having either a specified facial expression or a specified non-evocative feature; and
the one or more integration rules are configured such that the plurality of evocative elements are presented with at least two differing non-evocative features, each non-evocative feature either being correlated with a specific facial expression or not correlated with any facial expressions;
receive data indicative of the first response and the secondary response, at least one of the first response and the secondary response comprising a measure of the individual's response to the evocative elements, wherein (i) the response comprises a physical action of the individual in response to the evocative elements and (ii) the data comprises at least one measure of emotional processing capabilities of the individual under emotional load; and
analyze the data indicative of the first response and the secondary response to generate a first performance metric comprising at least one quantified indicator of cognitive abilities of the individual under emotional load;
adjust a difficulty of at least one at least one of the primary task or the interference based on the first performance metric such that the apparatus renders the primary task with the interference at a second difficulty level; and
generate a second performance metric representative of cognitive abilities of the individual under emotional load based at least in part on the data indicative of the first response and the second response from the second iteration to provide an indication of a difference in cognitive abilities of the individual.

38. The system of claim 36 or 37, wherein the one or more processors are further configured to render the first instance of the primary task and the interference to obtain the first and second responses in an iterative manner, with the difficulty level being adjusted between two or more of the iterations.

39. The system of claim 38, wherein adjusting the difficulty level comprises modifying a time-varying aspect of the first instance of the primary task and/or the interference.

40. The system of claim 39, wherein modifying the time-varying characteristics of an aspect of the primary task or the interference comprises adjusting a temporal length of the rendering of the task or interference at the user interface between two or more sessions of interactions of the individual.

41. The system of claim 39, wherein the time-varying characteristics is at least one of a speed of an evocative element, a rate of change of a facial expression, a direction of trajectory of an evocative element, a change of orientation of an evocative element, at least one color of an evocative element, a type of an evocative element, or a size of an evocative element.

42. The system of claim 41, wherein the change in type of evocative element is effected using morphing from a first type of evocative element to a second type of evocative element or rendering a blendshape as a proportionate combination of the first type of evocative element and the second type of evocative element.
